SEQUENCE LISTING (80 sequences)

SEQ ID NO: 1 - RNA, 2400 nt, Homo sapiens
  source 1..2400: mol_type = mRNA
gcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggcagcgctggagagcgaggtggcttcaataagcctggtggacccatggatgaaggaccagatcttgatctaggcccacctgtagatccagatgaagactctgacaacagtgcaatttatgtacaaggattaaatgacagtgtgactctagatgatctggcagacttctttaagcagtgtggggttgttaagatgaacaagagaactgggcaacccatgatccacatctacctggacaaggaaacaggaaagcccaaaggcgatgccacagtgtcctatgaagacccacccactgccaaggctgccgtggaatggtttgatgggaaagattttcaagggagcaaacttaaagtctcccttgctcggaagaagcctccaatgaacagtatgcggggtggtctgccaccccgtgagggcagaggcatgccaccaccactccgtggaggtccaggaggcccaggaggtcctgggggacccatgggtcgcatgggaggccgtggaggagatagaggaggcttccctccaagaggaccccggggttcccgagggaacccctctggaggaggaaacgtccagcaccgagctggagactggcagtgtcccaatccgggttgtggaaaccagaacttcgcctggagaacagagtgcaaccagtgtaaggccccaaagcctgaaggcttcctcccgccaccctttccgcccccgggtggtgatcgtggcagaggtggccctggtggcatgcggggaggaagaggtggcctcatggatcgtggtggtcccggtggaatgttcagaggtggccgtggtggagacagaggtggcttccgtggtggccggggcatggaccgaggtggctttggtggaggaagacgaggtggccctggggggccccctggacctttgatggaacagatgggaggaagaagaggaggacgtggaggacctggaaaaatggataaaggcgagcaccgtcaggagcgcagagatcggccctactagatgcagagaccccgcagagctgcattgactaccagatttattttttaaaccagaaaatgttttaaatttataattccatatttataatgttggccacaacattatgattattccttgtctgtactttagtatttttcaccatttgtgaagaaacattaaaacaagttaaatggtagtgtgcggagtttttttttcttccttcttttaaaaatggttgtttaagactttaacaatgggaaccccttgtgagcatgctcagtatcattgtggagaaccaagagggcctcttaactgtaacaatgttcatggttgtgatgttttttttttttttttaaataaaattccaaatgtttataaaga

SEQ ID NO: 2 - RNA, 2412 nt, Homo sapiens
  source 1..2412: mol_type = mRNA
agtagcggccctgactgccgaggaaacggtagcttaggacagttggctgttaagtgacactgattctccccgccccgcctgacccccgagaaggaggcttgtcccccgctgcgtgagggggtggggaagtgggtagtgaattcggatctacctgggaggggggagtggaagttcccgccccggagagcggcgaggcggcagccacagttgattatggaagattcccacaagagtaccacgtcagagacagcacctcaacctggttcagcagttcagggagctcacatttctcatattgctcaacaggtatcatctttatcagaaagtgaggagtcccaggactcatccgacagcataggctcctcacagaaagcccacgggatcctagcacggcgcccatcttacagaaaaattttgaaagacttatcttctgaagatacacggggcagaaaaggagacggagaaaattctggagtttctgctgctgtcacttctatgtctgttccaactcccatctatcagactagcagcggacagtacattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgattcctaagaaagaaaatatttttgtggacatgcataaaaattaaatggatttcctagtggagttttataaattaaaaggtcaaaactgaagctttttatttaggcttttccaaatcaaggataaatatcttacgcacgatatctagtgacagaggagaaagtggaaaatgacctcaaggaagctacgggcacaactggaagctttgtagaaattaaacatattcaaggagcaagaaatgaactttcagcagtctaaattttctaaataaccaatagttgccaatctaaaatggcagagaagatgaaatttgataaactgaattttttttaaaaatccatttaccctacaggtttgcatttgtttgctgaaatttacctttttttagttatatatatgtgtgtgtgtgtgtgtaatttctgccaataaattctaaattacaaaggtaagagaaaacctagtacattactaaatatataaagtatatgttctgattatgtatacttgttctagtgtcaagtctttttaagtgggtttttaaaagtttgttattggacttgaatggatttttgagactaggttaattatttttgaggtcttatcctaaaaggcatctaaggtacatgaatggagtatggtgattttataacattttttatcagaatggaaaaagaactgtttaaaagtttgatacttttaaatagttggtttttttgcttactctggtaatgattttctacaaatacataataaattgtttttttgagtctatattctgtatgcagttgaatatccattacttattctgctgtgctttaatagaatggaatgtttacaggcccttaaaatattatttttaaaaaaccttctgaagatacataccaaagtttttccaagaagattttataatcaatttaataatgtaaggtttatcagattctataatagtatagttattaaggcaattttatgttagagactattttgtaatgtagtgagtggtacctttataagaaaagtgactgccaatatatttttatagctgatctttataaattctaatgttgagtttttaatgattattttaaatgtttatatagtttagtaaaatttgcatctcaaagtatcatttttatattatgggacgttttcagattggctaatatttgcattgtaaattttgtatgcagtttatctaaaattcaaaaatactgtcagtacaccagcgtttaacatctatattccaatttgtatacagtttaaaattgtactgcaaaactattgtgtgctcttacacagtatgcatcatattgttgtctgtgaaattaaaggacatttgatagtctactgaatgtaaaatataatgcttggtatatgaatgataa

SEQ ID NO: 3 - RNA, 3051 nt, synthetic construct
  source 1..3051: mol_type = other RNA
gcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggcagcgctggagagcgaggtggcttcaataagcctggtggacccatggatgaaggaccagatcttgatctaggcccacctgtagatccagatgaagactctgacaacagtgcaatttatgtacaaggattaaatgacagtgtgactctagatgatctggcagacttctttaagcagtgtggggttgttaagatgaacaagagaactgggcaacccatgatccacatctacctggacaaggaaacaggaaagcccaaaggcgatgccacagtgtcctatgaagacccacccactgccaaggctgccgtggaatggtttgatgctgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgattcctaagaaagaaaatatttttgtggacatgcataaaaattaaatggatttcctagtggagttttataaattaaaaggtcaaaactgaagctttttatttaggcttttccaaatcaaggataaatatcttacgcacgatatctagtgacagaggagaaagtggaaaatgacctcaaggaagctacgggcacaactggaagctttgtagaaattaaacatattcaaggagcaagaaatgaactttcagcagtctaaattttctaaataaccaatagttgccaatctaaaatggcagagaagatgaaatttgataaactgaattttttttaaaaatccatttaccctacaggtttgcatttgtttgctgaaatttacctttttttagttatatatatgtgtgtgtgtgtgtgtaatttctgccaataaattctaaattacaaaggtaagagaaaacctagtacattactaaatatataaagtatatgttctgattatgtatacttgttctagtgtcaagtctttttaagtgggtttttaaaagtttgttattggacttgaatggatttttgagactaggttaattatttttgaggtcttatcctaaaaggcatctaaggtacatgaatggagtatggtgattttataacattttttatcagaatggaaaaagaactgtttaaaagtttgatacttttaaatagttggtttttttgcttactctggtaatgattttctacaaatacataataaattgtttttttgagtctatattctgtatgcagttgaatatccattacttattctgctgtgctttaatagaatggaatgtttacaggcccttaaaatattatttttaaaaaaccttctgaagatacataccaaagtttttccaagaagattttataatcaatttaataatgtaaggtttatcagattctataatagtatagttattaaggcaattttatgttagagactattttgtaatgtagtgagtggtacctttataagaaaagtgactgccaatatatttttatagctgatctttataaattctaatgttgagtttttaatgattattttaaatgtttatatagtttagtaaaatttgcatctcaaagtatcatttttatattatgggacgttttcagattggctaatatttgcattgtaaattttgtatgcagtttatctaaaattcaaaaatactgtcagtacaccagcgtttaacatctatattccaatttgtatacagtttaaaattgtactgcaaaactattgtgtgctcttacacagtatgcatcatattgttgtctgtgaaattaaaggacatttgatagtctactgaatgtaaaatataatgcttggtatatgaatgataa

SEQ ID NO: 4 - protein, 532 aa, synthetic construct
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQSSFRQDHPSSMGVYGQESGGFSGPGENRSMSGPDNRGRGRGGFDRGGMSRGGRGGGRGGMGSAGERGGFNKPGGPMDEGPDLDLGPPVDPDEDSDNSAIYVQGLNDSVTLDDLADFFKQCGVVKMNKRTGQPMIHIYLDKETGKPKGDATVSYEDPPTAKAAVEWFDAASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

SEQ ID NO: 5 - RNA, 3046 nt, synthetic construct
  source 1..3046: mol_type = other RNA
gcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggaaaaattttgaaagacttatcttctgaagatacacggggcagaaaaggagacggagaaaattctggagtttctgctgctgtcacttctatgtctgttccaactcccatctatcagactagcagcggacagtacattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgattcctaagaaagaaaatatttttgtggacatgcataaaaattaaatggatttcctagtggagttttataaattaaaaggtcaaaacgaagctttttatttaggcttttccaaatcaaggataaatatcttacgcacgatatctagtgacagaggagaaagtggaaaatgacctcaaggaagctacgggcacaactggaagctttgtagaaattaaacatattcaaggagcaaaaatgaactttcagcagtctaaattttctaaataaccaatagttgccaatctaaaatggcagagaagatgaaatttgataaactgaattttttttaaaaatccatttaccctacaggtttgcatttgtttgctgaaatttacctttttttagttatatatatgtgtgtgtgtgtgtgtaatttctgccaataaattctaaattacaaaggtaagagaaaacctagtacattactaaatatataaagtatatgttctgattatgtatacttgttctagtgtcaagtctttttaagtgggtttttaaaagtttgttattggacttgaatggatttttgagactaggttaattatttttgaggtcttatcctaaaaggcatctaaggtacatgaatggagtatggtgattttataacattttttatcagaatggaaaaagaactgtttaaaagtttgatacttttaaatagttggtttttttgcttactctggtaatgattttctacaaatacataataaattgtttttttgagtctatattctgtatgcagttgaatatccattacttattctgctgtgctttaatagaatggaatgtttacaggcccttaaaatattatttttaaaaaaccttctgaagatacataccaaagtttttccaagaagattttataatcaatttaataatgtaaggtttatcagattctataatagtatagttattaaggcaattttatgttagagactattttgtaatgtagtgagtggtacctttataagaaaagtgactgccaatatatttttatagctgatctttataaattctaatgttgagtttttaatgattattttaaatgtttatatagtttagtaaaatttgcatctcaaagtatcatttttatattatgggacgttttcagattggctaatatttgcattgtaaattttgtatgcagtttatctaaaattcaaaaatactgtcagtacaccagcgtttaacatctatattccaatttgtatacagtttaaaattgtactgcaaaactattgtgtgctcttacacagtatgcatcatattgttgtctgtgaaattaaaggacatttgatagtctactgaatgtaaaatataatgcttggtatatgaatgataa

SEQ ID NO: 6 - protein, 531 aa, synthetic construct
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQSSFRQDHPSSMGVYGQESGGFSGPGENRSMSGPDNRGRGRGGFDRGGMSRGGRGGGRGGMGKILKDLSSEDTRGRKGDGENSGVSAAVTSMSVPTPIYQTSSGQYIAIAPNGALQLASPGTDGVQGLQTLTMTNSGSTQQGTTILQYAQTSDGQQILVPSNQVVVQTASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

SEQ ID NO: 7 - RNA, 2733 nt, synthetic construct
  source 1..2733: mol_type = other RNA
gcagtgcggcgcctagagggaaagcgagagggagacggacgttgagagaacgaggaggaaggagagaaaatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgattcctaagaaagaaaatatttttgtggacatgcataaaaattaaatggatttcctagtggagttttataaattaaaaggtcaaaactgaagctttttatttaggcttttccaaatcaaggataaatatcttacgcacgatatctagtgacagaggagaaagtggaaaatgacctcaaggaagctacgggcacaactggaagctttgtagaaattaaacatattcaaggagcaagaaatgaactttcagcagtctaaattttctaaataaccaatagttgccaatctaaaatggcagagaagatgaaatttgataaactgaattttttttaaaaatccatttaccctacaggtttgcatttgtttgctgaaatttacctttttttagttatatatatgtgtgtgtgtgtgtgtaatttctgccaataaattctaaattacaaaggtaagagaaaacctagtacattactaaatatataaagtatatgttctgattatgtatacttgttctagtgtcaagtctttttaagtgggtttttaaaagtttgttattggacttgaatggatttttgagactaggttaattatttttgaggtcttatcctaaaaggcatctaaggtacatgaatggagtatggtgattttataacattttttatcagaatggaaaaagaactgtttaaaagtttgatacttttaaatagttggtttttttgcttactctggtaatgattttctacaaatacataataaattgtttttttgagtctatattctgtatgcagttgaatatccattacttattctgctgtgctttaatagaatggaatgtttacaggcccttaaaatattatttttaaaaaaccttctgaagatacataccaaagtttttccaagaagattttataatcaatttaataatgtaaggtttatcagattctataatagtatagttattaaggcaattttatgttagagactattttgtaatgtagtgagtggtacctttataagaaaagtgactgccaatatatttttatagctgatctttataaattctaatgttgagtttttaatgattattttaaatgtttatatagtttagtaaaatttgcatctcaaagtatcatttttatattatgggacgttttcagattggctaatatttgcattgtaaattttgtatgcagtttatctaaaattcaaaaatactgtcagtacaccagcgtttaacatctatattccaatttgtatacagtttaaaattgtactgcaaaactattgtgtgctcttacacagtatgcatcatattgttgtctgtgaaattaaaggacatttgatagtctactgaatgtaaaatataatgcttggtatatgaatgataa

SEQ ID NO: 8 - protein, 426 aa, synthetic construct
MASTDYSTYSQAAAQQGYSAYTAQPTQGYAQTTQAYGQQSYGTYGQPTDVSYTQAQTTATYGQTAYATSYGQPPTGYTTPTAPQAYSQPVQGYGTGAYDTTTATVTTTQASYAAQSAYGTQPAYPAYGQQPAATAPTRPQDGNKPTETSQPQSSTGGYNQPSLGYGQSNYSYPQVPGSYPMQPVTAPPSYPPTSYSSTQPTSYDQSSYSQQNTYGQPSSYGQQSSYGQQSSYGQQPPTSYPPQTGSYSQAPSQYSQQSSSYGQQIAIAPNGALQLASPGTDGVQGLQTLTMTNSGSTQQGTTILQYAQTSDGQQILVPSNQVVVQTASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

SEQ ID NO: 9 - DNA, 5607 bp, synthetic construct
  source 1..5607: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgccaccatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggcagcgctggagagcgaggtggcttcaataagcctggtggacccatggatgaaggaccagatcttgatctaggcccacctgtagatccagatgaagactctgacaacagtgcaatttatgtacaaggattaaatgacagtgtgactctagatgatctggcagacttctttaagcagtgtggggttgttaagatgaacaagagaactgggcaacccatgatccacatctacctggacaaggaaacaggaaagcccaaaggcgatgccacagtgtcctatgaagacccacccactgccaaggctgccgtggaatggtttgatgctgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 10 - DNA, 5640 bp, synthetic construct
  source 1..5640: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgccaccatgtggagccatcctcagtttgagaaaggttctatggcgtccacggattacagtacctatagccaagctgcagcgcagcagggctacagtgcttacaccgcccagcccactcaaggatatgcacagaccacccaggcatatgggcaacaaagctatggaacctatggacagcccactgatgtcagctatacccaggctcagaccactgcaacctatgggcagaccgcctatgcaacttcttatggacagcctcccactggttatactactccaactgccccccaggcatacagccagcctgtccaggggtatggcactggtgcttatgataccaccactgctacagtcaccaccacccaggcctcctatgcagctcagtctgcatatggcactcagcctgcttatccagcctatgggcagcagccagcagccactgcacctacaagaccgcaggatggaaacaagcccactgagactagtcaacctcaatctagcacagggggttacaaccagcccagcctaggatatggacagagtaactacagttatccccaggtacctgggagctaccccatgcagccagtcactgcacctccatcctaccctcctaccagctattcctctacacagccgactagttatgatcagagcagttactctcagcagaacacctatgggcaaccgagcagctatggacagcagagtagctatggtcaacaaagcagctatgggcagcagcctcccactagttacccaccccaaactggatcctacagccaagctccaagtcaatatagccaacagagcagcagctacgggcagcagagttcattccgacaggaccaccccagtagcatgggtgtttatgggcaggagtctggaggattttccggaccaggagagaaccggagcatgagtggccctgataaccggggcaggggaagagggggatttgatcgtggaggcatgagcagaggtgggcggggaggaggacgcggtggaatgggcagcgctggagagcgaggtggcttcaataagcctggtggacccatggatgaaggaccagatcttgatctaggcccacctgtagatccagatgaagactctgacaacagtgcaatttatgtacaaggattaaatgacagtgtgactctagatgatctggcagacttctttaagcagtgtggggttgttaagatgaacaagagaactgggcaacccatgatccacatctacctggacaaggaaacaggaaagcccaaaggcgatgccacagtgtcctatgaagacccacccactgccaaggctgccgtggaatggtttgatgctgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttgagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 11 - RNA, 4527 nt, synthetic construct
  source 1..4527: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcgtggaatggtttgatgctgcatagatatgtgcatctatgcagcatcaaaccattccacgcattatggtgacagctgcctcgggaagccaagttgggctttaaagtgcagggcctgctgatgttgagtgctttttgttccgtgtcatgcagagatgcttgatagtgaagtagattagcatctatgcagcatcaaaccattccacgcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 12 - RNA, 4527 nt, synthetic construct
  source 1..4527: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 13 - RNA, 4527 nt, synthetic construct
  source 1..4527: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgccgtggaatggtttgatgctgcagatatgtgcatctgcagcatcaaaccattccacggccattatggtgacagctgcctcgggaagccaagttgggctttaaagtgcagggcctgctgatgttgagtgctttttgttcgctatgtcatacattgatgacgcagtgaagtagattagcatctgcagcatcaaaccattccacggccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 14 - RNA, 4527 nt, synthetic construct
  source 1..4527: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgttgccgtggaatggtttgatgctgagatatgtgcatctcagcatcaaaccattccacggcacattatggtgacagctgcctcgggaagccaagttgggctttaaagtgcagggcctgctgatgttgagtgctttttgttctgatgtgtaacaatttgacgatgagtgaagtagattagcatctcagcatcaaaccattccacggcacataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 15 - RNA, 4527 nt, synthetic construct
  source 1..4527: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgccgtggaatggtttgatgctagatatgtgcatctagcatcaaaccattccacggcagcattatggtgacagctgcctcgggaagccaagttgggctttaaagtgcagggcctgctgatgttgagtgctttttgttcctgatgtggatcagtttgacactagtgaagtagattagcatctagcatcaaaccattccacggcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 16 - RNA, 4257 nt, synthetic construct
  source 1..4257: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtcgtcgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgttgccgtggaatggtttgatgctgagatatgtgcatctcagcatcaaaccattccacggcacattatggtgacagctgcctcgggaagccaagttgggctttaaagtgcagggcctgctgatgttgagtgctttttgttctgatgtgtaacaatttgacgatgagtgaagtagattagcatctcagcatcaaaccattccacggcacataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 17 - RNA, 5105 nt, synthetic construct
  source 1..5105: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttaggacgcggtggaatgggaaaaaagaagaatgtagttttttcccattccaccgcgtccttggtggcctgctatttccttcaaatgaatgatttttactaattttgtgtacttttattgtgtcgatgtagaatctgcctggtctatctgatgtgacagcttctgtagcacaggacgatgaacaatgggtgaaatgtttagttatcttttttcccattccaccgcgtccttactgctagctgtagaactccagcttcggcctgtcgcccaatcaaactgtcctgttactgaacactgttctatggttgcagctacgggcagcagattgcctgtgtgatattctgcggcaatctgctgcccgtagctgcctgtggtagtgaaaagtctgtagaaaagtaagggaaactcaaacccctttctacacgcagacacgagtagcagatcaccgtgtttctgtatggggcaatctgctgcccgtagctgcttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 18 - RNA, 4835 nt, synthetic construct
  source 1..4835: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtcgtcgactcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttaggacgcggtggaatgggaaaaaagaagaatgtagttttttcccattccaccgcgtccttggtggcctgctatttccttcaaatgaatgatttttactaattttgtgtacttttattgtgtcgatgtagaatctgcctggtctatctgatgtgacagcttctgtagcacaggacgatgaacaatgggtgaaatgtttagttatcttttttcccattccaccgcgtccttactgctagctgtagaactccagcttcggcctgtcgcccaatcaaactgtcctgttactgaacactgttctatggttgcagctacgggcagcagattgcctgtgtgatattctgcggcaatctgctgcccgtagctgcctgtggtagtgaaaagtctgtagaaaagtaagggaaactcaaacccctttctacacgcagacacgagtagcagatcaccgtgtttctgtatggggcaatctgctgcccgtagctgcttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 19 - RNA, 5105 nt, synthetic construct
  source 1..5105: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgactcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttgaggacgcggtggaatgggaaaaagaagaatgtagtttttcccattccaccgcgtcctctggtggcctgctatttccttcaaatgaatgatttttactaattttgtgtacttttattgtgtcgatgtagaatctgcctggtctatctgatgtgacagcttctgtagcacgagttagcgagtgaatggtcaaatgtttagttatctttttcccattccaccgcgtcctctactgctagctgtagaactccagcttcggcctgtcgcccaatcaaactgtcctgttactgaacactgttctatggttagcagctacgggcagcagattgctgtgtgatattctgcgcaatctgctgcccgtagctgctctgtggtagtgaaaagtctgtagaaaagtaagggaaactcaaacccctttctacacagcatacacatacagcatcatgcgtgtttctgtatgggcaatctgctgcccgtagctgctttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 20 - RNA, 4835 nt, synthetic construct
  source 1..4835: mol_type = other RNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtcgtcgactcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttgaggacgcggtggaatgggaaaaagaagaatgtagtttttcccattccaccgcgtcctctggtggcctgctatttccttcaaatgaatgatttttactaattttgtgtacttttattgtgtcgatgtagaatctgcctggtctatctgatgtgacagcttctgtagcacgagttagcgagtgaatggtcaaatgtttagttatctttttcccattccaccgcgtcctctactgctagctgtagaactccagcttcggcctgtcgcccaatcaaactgtcctgttactgaacactgttctatggttagcagctacgggcagcagattgctgtgtgatattctgcgcaatctgctgcccgtagctgctctgtggtagtgaaaagtctgtagaaaagtaagggaaactcaaacccctttctacacagcatacacatacagcatcatgcgtgtttctgtatgggcaatctgctgcccgtagctgctttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatattttttgcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 21 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcgtggaatggtttgatgctgcatagatatgtgcatctatgcagcatcaaaccattccacgcattatggtgacagctgcctcgggaagccaagttgggctttaaagtgcagggcctgctgatgttgagtgctttttgttccgtgtcatgcagagatgcttgatagtgaagtagattagcatctatgcagcatcaaaccattccacgcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 22 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 23 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgccgtggaatggtttgatgctgcagatatgtgcatctgcagcatcaaaccattccacggccattatggtgacagctgcctcgggaagccaagttgggctttaaagtgcagggcctgctgatgttgagtgctttttgttcgctatgtcatacattgatgacgcagtgaagtagattagcatctgcagcatcaaaccattccacggccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 24 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgttgccgtggaatggtttgatgctgagatatgtgcatctcagcatcaaaccattccacggcacattatggtgacagctgcctcgggaagccaagttgggctttaaagtgcagggcctgctgatgttgagtgctttttgttctgatgtgtaacaatttgacgatgagtgaagtagattagcatctcagcatcaaaccattccacggcacataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 25 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgccgtggaatggtttgatgctagatatgtgcatctagcatcaaaccattccacggcagcattatggtgacagctgcctcgggaagccaagttgggctttaaagtgcagggcctgctgatgttgagtgctttttgttcctgatgtggatcagtttgacactagtgaagtagattagcatctagcatcaaaccattccacggcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 26 - RNA, 552 nt, synthetic construct; identical to SEQ ID NO: 24
  source 1..552: mol_type = other RNA

SEQ ID NO: 27 - RNA, 1130 nt, synthetic construct
  source 1..1130: mol_type = other RNA
tcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttaggacgcggtggaatgggaaaaaagaagaatgtagttttttcccattccaccgcgtccttggtggcctgctatttccttcaaatgaatgatttttactaattttgtgtacttttattgtgtcgatgtagaatctgcctggtctatctgatgtgacagcttctgtagcacaggacgatgaacaatgggtgaaatgtttagttatcttttttcccattccaccgcgtccttactgctagctgtagaactccagcttcggcctgtcgcccaatcaaactgtcctgttactgaacactgttctatggttgcagctacgggcagcagattgcctgtgtgatattctgcggcaatctgctgcccgtagctgcctgtggtagtgaaaagtctgtagaaaagtaagggaaactcaaacccctttctacacgcagacacgagtagcagatcaccgtgtttctgtatggggcaatctgctgcccgtagctgcttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatatttttt

SEQ ID NO: 28 - RNA, 1130 nt, synthetic construct; identical to SEQ ID NO: 27
  source 1..1130: mol_type = other RNA

SEQ ID NO: 29 - RNA, 1130 nt, synthetic construct
  source 1..1130: mol_type = other RNA
tcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcatgatatgtgcatcttgcagcatcaaaccattccacggcattatggtgacagctgcctcgggaagccaagttgggctttaaagtgcagggcctgctgatgttgagtgctttttgttcccatgtaatgacctgatgatacatgaagtagattagcatcttgcagcatcaaaccattccacggcataagaagttatgtattcatccaataattcaagccaagcaagtatataggtgttttaatagtttttgtttgcagtcctctgttgaggacgcggtggaatgggaaaaagaagaatgtagtttttcccattccaccgcgtcctctggtggcctgctatttccttcaaatgaatgatttttactaattttgtgtacttttattgtgtcgatgtagaatctgcctggtctatctgatgtgacagcttctgtagcacgagttagcgagtgaatggtcaaatgtttagttatctttttcccattccaccgcgtcctctactgctagctgtagaactccagcttcggcctgtcgcccaatcaaactgtcctgttactgaacactgttctatggttagcagctacgggcagcagattgctgtgtgatattctgcgcaatctgctgcccgtagctgctctgtggtagtgaaaagtctgtagaaaagtaagggaaactcaaacccctttctacacagcatacacatacagcatcatgcgtgtttctgtatgggcaatctgctgcccgtagctgctttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttttcattcaaaaacatttcacttttggggttgcgtgtcagatttggcagtataaattctggctatatttttt

SEQ ID NO: 30 - RNA, 1130 nt, synthetic construct; identical to SEQ ID NO: 29
  source 1..1130: mol_type = other RNA

SEQ ID NO: 31 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtaggacgcggtggaatgggaaaaaagatatgtgcatcttttttcccattccaccgcgtcctcattatggtgacagctgcctcgggaagccaagttgggctttaaagtgcagggcctgctgatgttgagtgctttttgttcaggacgatgaacaatgggtgaaaagtgaagtagattagcatcttttttcccattccaccgcgtcctcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 32 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 33 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 34 - RNA, 552 nt, synthetic construct
  source 1..552: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 35 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtaggacgcggtggaatgggaaaaaagatatgtgcatcttttttcccattccaccgcgtcctcattatggtgacagctgcctcgggaagccaagttgggctttaaagtgcagggcctgctgatgttgagtgctttttgttcaggacgatgaacaatgggtgaaaagtgaagtagattagcatcttttttcccattccaccgcgtcctcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 36 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtaggacgcggtggaatgggaaaaaagatatgtgcatcttttttcccattccaccgcgtcctcattatggtgacagctgcctcgggaagccaagttgggctttaaagtgcagggcctgctgatgttgagtgctttttgttcaggacgatgaacaatgggtgaaaagtgaagtagattagcatcttttttcccattccaccgcgtcctcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 37 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtaggacgcggtggaatgggaaaaaagatatgtgcatcttttttcccattccaccgcgtcctcattatggtgacagctgcctcgggaagccaagttgggctttaaagtgcagggcctgctgatgttgagtgctttttgttcaggacgatgaacaatgggtgaaaagtgaagtagattagcatcttttttcccattccaccgcgtcctcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 38 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 39 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 40 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 41 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtaggacgcggtggaatgggaaaaaagatatgtgcatcttttttcccattccaccgcgtcctcattatggtgacagctgcctcgggaagccaagttgggctttaaagtgcagggcctgctgatgttgagtgctttttgttcaggacgatgaacaatgggtgaaaagtgaagtagattagcatcttttttcccattccaccgcgtcctcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 42 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 43 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 44 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtccgtggaatggtttgatgctgcaagatatgtgcatcttgcagcatcaaaccattccacggcattatggtgacagctgcctcgggaagccaagttgggctttaaagtgcagggcctgctgatgttgagtgctttttgttcccatgtaatgacctgatgatacaagtgaagtagattagcatcttgcagcatcaaaccattccacggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 45 - RNA, 552 nt, synthetic construct; identical to SEQ ID NO: 32
  source 1..552: mol_type = other RNA

SEQ ID NO: 46 - RNA, 1104 nt, synthetic construct; identical to SEQ ID NO: 35
  source 1..1104: mol_type = other RNA

SEQ ID NO: 47 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 48 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgaccagatcttgatctagttgccagatatgtgcatctggcaactagatcaagatctggtccattatggtgacagctgcctcgggaagccaagttgggctttaaagtgcagggcctgctgatgttgagtgctttttgttcgacaggatcgatatctagtcaccagtgaagtagattagcatctggcaactagatcaagatctggtccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 49 - RNA, 1104 nt, synthetic construct
  source 1..1104: mol_type = other RNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagaagaagagatatgtgcatctcttcttctgctgcccgtagctgccattatggtgacagctgcctcgggaagccaagttgggctttaaagtgcagggcctgctgatgttgagtgctttttgttcgcacatacgatgagcagactaagagtgaagtagattagcatctcttcttctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtgcagctacgggcagcagattgccagatatgtgcatctggcaatctgctgcccgtagctgccattatggtgacagctgcctcgggaagccaagttgggctttaaagtgcagggcctgctgatgttgagtgctttttgttcgcagacacgagtagcagatcaccagtgaagtagattagcatctggcaatctgctgcccgtagctgccataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 50 - RNA, 1104 nt, synthetic construct; identical to SEQ ID NO: 42
  source 1..1104: mol_type = other RNA

SEQ ID NO: 51 - DNA, 24 bp, synthetic construct
  source 1..24: mol_type = other DNA
cgtggaatggtttgatgctgcatc

SEQ ID NO: 52 - DNA, 24 bp, synthetic construct
  source 1..24: mol_type = other DNA
ccgtggaatggtttgatgctgcat

SEQ ID NO: 53 - DNA, 24 bp, synthetic construct
  source 1..24: mol_type = other DNA
gccgtggaatggtttgatgctgca

SEQ ID NO: 54 - DNA, 24 bp, synthetic construct
  source 1..24: mol_type = other DNA
tgccgtggaatggtttgatgctgc

SEQ ID NO: 55 - DNA, 24 bp, synthetic construct
  source 1..24: mol_type = other DNA
ctgccgtggaatggtttgatgctg

SEQ ID NO: 56 - DNA, 24 bp, synthetic construct
  source 1..24: mol_type = other DNA
tgccgtggaatggtttgatgctgc

SEQ ID NO: 57 - DNA, 248 bp, synthetic construct
  source 1..248: mol_type = other DNA
agagaactgggcaacccatgatccacatctacctggacaaggaaacaggaaagcccaaaggcgatgccacagtgtcctatgaagacccacccactgccaaggctgccgtggaatggtttgatgctgcatcaggagatatgcaaacatatcagatccgaactacaccttcagctacttctctgccacaaactgtggtgatgacatctcctgtgactctcacctctcagacaactaagacagatgacccc

SEQ ID NO: 58 - DNA, 280 bp, synthetic construct
  source 1..280: mol_type = other DNA
atccacatctacctggacaaggaaacaggaaagcccaaaggcgatgccacagtgtcctatgaagacccacccactgccaaggctgccgtggaatggtttgatgggaaagattttcaagggagcaaacttaaagtctcccttgctcggaagaagcctccaatgaacagtatgcggggtggtctgccaccccgtgagggcagaggcatgccaccaccactccgtggaggtccaggaggcccaggaggtcctgggggacccatgggtcgcatgggaggccgtg

SEQ ID NO: 59 - DNA, 222 bp, synthetic construct
  source 1..222: mol_type = other DNA
cctgataaccggggcaggggaagagggggatttgatcgtggaggcatgagcagaggtgggcggggaggaggacgcggtggaatgggaaaaattttgaaagacttatcttctgaagatacacggggcagaaaaggagacggagaaaattctggagtttctgctgctgtcacttctatgtctgttccaactcccatctatcagactagcagcggacagtacatt

SEQ ID NO: 60 - DNA, 222 bp, synthetic construct
  source 1..222: mol_type = other DNA
tagctatggtcaacaaagcagctatgggcagcagcctcccactagttacccaccccaaactggatcctacagccaagctccaagtcaatatagccaacagagcagcagctacgggcagcagattgccattgccccaaatggagccttacagttggcaagtccaggcacagatggagtacagggacttcagacattaaccatgacaaattcaggcagtactca

SEQ ID NO: 61 - DNA, 222 bp, synthetic construct
  source 1..222: mol_type = other DNA
tgatccacatctacctggacaaggaaacaggaaagcccaaaggcgatgccacagtgtcctatgaagacccacccactgccaaggctgccgtggaatggtttgatgctgcatcaggagatatgcaaacatatcagatccgaactacaccttcagctacttctctgccacaaactgtggtgatgacatctcctgtgactctcacctctcagacaactaagacag

SEQ ID NO: 62 - RNA, 1050 nt, synthetic construct
  source 1..1050: mol_type = other RNA
cccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcaaagtgcttacagtgcaggtagtgatatgtgcatctactgcagtgaaggcacttgtagcattatggtgacagctgcctcgggaagccaagttgggctttaaagtgcagggcctgctgatgttgagtgctttttgttctaaggtgcatctagtgcagatagtgaagtagattagcatctactgccctaagtgctccttctggcataagaagttatgtattcatccaataattcaagccaagcaagtatataggtgttttaatagtttttgtttgcagtcctctgttagttttgcatagttgcactacaagaagaatgtagttgtgcaaatctatgcaaaactgatggtggcctgctatttccttcaaatgaatgatttttactaattttgtgtacttttattgtgtcgatgtagaatctgcctggtctatctgatgtgacagcttctgtagcactaaagtgcttatagtgcaggtagtgtttagttatctactgcattatgagcacttaaagtactgctagctgtagaactccagcttcggcctgtcgcccaatcaaactgtcctgttactgaacactgttctatggttagttttgcaggtttgcatccagctgtgtgatattctgctgtgcaaatccatgcaaaactgactgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatacatgaaatattaaagaaaatgtgtaactttttgtgtaaatacatcttgtcttgttt

SEQ ID NO: 63 - RNA, 24 nt, synthetic construct
  source 1..24: mol_type = other RNA
caaagtgcttacagtgcaggtagt

SEQ ID NO: 64 - RNA, 23 nt, synthetic construct
  source 1..23: mol_type = other RNA
taaagtgcttatagtgcaggtag

SEQ ID NO: 65 - RNA, 22 nt, synthetic construct
  source 1..22: mol_type = other RNA
taaggtgcatctagtgcagata

SEQ ID NO: 66 - RNA, 23 nt, synthetic construct
  source 1..23: mol_type = other RNA
tgtgcaaatctatgcaaaactga

SEQ ID NO: 67 - RNA, 23 nt, synthetic construct
  source 1..23: mol_type = other RNA
tgtgcaaatccatgcaaaactga

SEQ ID NO: 68 - RNA, 21 nt, synthetic construct
  source 1..21: mol_type = other RNA
tattgcacttgtcccggcctg

SEQ ID NO: 69 - RNA, 24 nt, synthetic construct
  source 1..24: mol_type = other RNA
caaagtgcttacagtgcaggtagt

SEQ ID NO: 70 - RNA, 23 nt, synthetic construct
  source 1..23: mol_type = other RNA
taaagtgcttatagtgcaggtag

SEQ ID NO: 71 - RNA, 22 nt, synthetic construct
  source 1..22: mol_type = other RNA
taaggtgcatctagtgcagata

SEQ ID NO: 72 - RNA, 23 nt, synthetic construct
  source 1..23: mol_type = other RNA
tgtgcaaatctatgcaaaactga

SEQ ID NO: 73 - RNA, 23 nt, synthetic construct
  source 1..23: mol_type = other RNA
tgtgcaaatccatgcaaaactga

SEQ ID NO: 74 - RNA, 21 nt, synthetic construct
  source 1..21: mol_type = other RNA
tattgcacttgtcccggcctg

SEQ ID NO: 75 - RNA, 1130 nt, synthetic construct; identical to SEQ ID NO: 27
  source 1..1130: mol_type = other RNA

SEQ ID NO: 76 - RNA, 1130 nt, synthetic construct; identical to SEQ ID NO: 29
  source 1..1130: mol_type = other RNA

SEQ ID NO: 77 - DNA, 23 bp, synthetic construct
  source 1..23: mol_type = other DNA
aggacgcggtggaatgggaaaaa

SEQ ID NO: 78 - DNA, 23 bp, synthetic construct
  source 1..23: mol_type = other DNA
gaggacgcggtggaatgggaaaa

SEQ ID NO: 79 - DNA, 23 bp, synthetic construct
  source 1..23: mol_type = other DNA
gcagctacgggcagcagattgcc

SEQ ID NO: 80 - DNA, 23 bp, synthetic construct
  source 1..23: mol_type = other DNA
agcagctacgggcagcagattgc

(19) United States
(12) Patent Application Publication
(10) Pub. No.: US 2026/0146248 A1
(43) Pub. Date: May 28, 2026

(54) COMPOSITION AND METHOD FOR TREATING CANCER WITH CHROMOSOME TRANSLOCATION FUSION

(71) Applicant: Gradalis, Inc., Carrollton, TX (US)

(72) Inventors: Donald D. Rao, Dallas, TX (US); John J. Nemunaitis, Dallas, TX (US); Fabienne Kerneis, Plano, TX (US)

(21) Appl. No.: 19/398,169

(22) Filed: Nov. 24, 2025

Related U.S. Application Data
(63) Continuation of application No. PCT/US2025/056560, filed on Nov. 21, 2025.
(60) Provisional application No. 63/724,560, filed on Nov. 25, 2024.

(51) Int. Cl. C12N 15/113; A61K 48/00; A61P 35/00; C12N 15/85

(52) U.S. Cl. CPC C12N 15/1135; A61K 48/0041; A61P 35/00; C12N 15/85; C12N 2310/14; C12N 2310/531; C12N 2800/107

(57) ABSTRACT

Compositions and methods for knocking down the expression of an EWSR1-ATF1 fusion gene and/or an EWSR1-CREB1 fusion gene in a subject are disclosed herein. In some embodiments, the composition comprises an expression vector comprising a bifunctional short hairpin RNA (bi-shRNA) sequence specific for knockdown of an EWSR1-ATF1 fusion gene and/or an EWSR1-CREB1 fusion gene.

### CROSS-REFERENCES TO RELATED APPLICATIONS

This application is a continuation application of PCT Application No. PCT/US2025/056560, filed Nov. 21, 2025, which claims benefit of U.S. Provisional Patent Application No. 63/724,560, filed Nov. 25, 2024, which applications are incorporated herein by reference in their entirety.

### REFERENCE TO AN ELECTRONIC SEQUENCE LISTING

The contents of the electronic sequence listing (097999-1531312-004410US_SL.xml; Size: 175,377 bytes; and Date of Creation: Nov. 12, 2025) is herein incorporated by reference in its entirety.

### BACKGROUND

Chromosome translocations aberrantly generate novel chromosomes and have been considered as the primary cause and the driver mutation oncogenes of several tumor types. The EWSR1 (Ewing sarcoma breakpoint region 1) gene, encoding the Ewing Sarcoma protein or the EWS protein and located on chromosome 22q12, is a member of the TET family, a highly conserved group of multifunctional, RNA-binding proteins. More than 90% of clear cell sarcomas are genetically characterized by a recurrent translocation involving the EWSR1 gene. The most common EWSR1 translocation partner for clear cell sarcoma is the activating transcription factor-1 (ATF1) gene on chromosome 12q13. EWSR1-ATF1 translocations are the key genetic factors for oncogenesis of a subset of clear cell sarcoma. A second less common translocation partner is CREB1 located on chromosome 2q34, which results in a rearrangement t(2;22) that has been preferentially reported in clear cell sarcomas of the gastrointestinal tract and not in clear cell sarcomas of the soft tissue, until recently (Hisaoka et al., Am J Surg Pathol 2008; 32:452-460). There are no current therapies that directly target EWSR1 fusion proteins.

### SUMMARY OF THE DISCLOSURE

In one aspect, the disclosure provides an expression vector comprising a bifunctional short hairpin RNA (bi-shRNA) sequence specific for knockdown of at least one EWSR1-ATF1 fusion gene and/or an EWSR1-CREB1 fusion gene, wherein the bi-shRNA sequence encodes a nucleic acid sequence capable of hybridizing to a target region of an mRNA transcript encoding the EWSR1-ATF1 fusion gene and/or a target region of an mRNA transcript encoding the EWSR1-CREB1 fusion gene to inhibit the expression of the EWSR1-ATF1 fusion gene and/or the EWSR1-CREB1 fusion gene via RNA interference, wherein the bi-shRNA comprises a first stem-loop structure that comprises an siRNA component and a second stem-loop structure that comprises a miRNA component.

In some embodiments, the siRNA component functions in a cleavage-dependent manner and the miRNA component functions in a cleavage-independent manner. In some embodiments, the bi-shRNA sequence is operably linked to a promoter. In certain embodiments, the promoter is a CMV mammalian promoter.

In some embodiments, the bi-shRNA is specific for knockdown of at least one EWSR1-ATF1 fusion gene.

In some embodiments, the bi-shRNA sequence is specific for knockdown of one EWSR1-ATF1 fusion gene. In certain embodiments, the bi-shRNA sequence comprises a sequence having at least 90% (e.g., 90%, 91%, 92%, 93%, 94%, 95%, 96%, 97%, 98%, 99%, or 100%) identity to any one of SEQ ID NOS:21 to 26.

In some embodiments, the bi-shRNA sequence is specific for knockdown of two EWSR1-ATF1 fusion genes. In certain embodiments, the bi-shRNA sequence comprises a sequence having at least 90% (e.g., 90%, 91%, 92%, 93%, 94%, 95%, 96%, 97%, 98%, 99%, or 100%) identity to any one of SEQ ID NOS:35 to 44.

In some embodiments, the bi-shRNA sequence is specific for knockdown of three EWSR1-ATF1 fusion genes. In certain embodiments, the bi-shRNA sequence comprises a sequence having at least 90% (e.g., 90%, 91%, 92%, 93%, 94%, 95%, 96%, 97%, 98%, 99%, or 100%) identity to any one of SEQ ID NOS:27-30.

In some embodiments, the bi-shRNA is specific for knockdown of an EWSR1-CREB1 fusion gene. In certain embodiments, the bi-shRNA sequence comprises a sequence having at least 90% (e.g., 90%, 91%, 92%, 93%, 94%, 95%, 96%, 97%, 98%, 99%, or 100%) identity to SEQ ID NO:45.

In some embodiments, the bi-shRNA is specific for knockdown of one EWSR1-ATF1 fusion gene and one EWSR1-CREB1 fusion gene. In certain embodiments, the bi-shRNA sequence comprises a sequence having at least 90% (e.g., 90%, 91%, 92%, 93%, 94%, 95%, 96%, 97%, 98%, 99%, or 100%) identity to any one of SEQ ID NOS:46 to 50.

In another aspect, the disclosure provides a composition comprising a therapeutic agent carrier and an expression vector described herein. In certain embodiments, the therapeutic agent carrier is a compacted DNA nanoparticle. In certain embodiments, the DNA nanoparticle is compacted with one or more polycations. In certain embodiments, the compacted DNA nanoparticles are further encapsulated in a liposome. In certain embodiments, the therapeutic agent carrier is a liposome. In certain embodiments, the liposome is a bilamellar invaginated vesicle (BIV). In some embodiments, the liposome is a reversibly masked liposome. In some embodiments, the liposome is decorated with one or more receptor targeting moieties. In some embodiments, the liposome comprises DOTAP and cholesterol.

In another aspect, the disclosure provides a method for delivering one or more bi-shRNAs to a target tissue expressing at least one EWSR1-ATF1 fusion gene and/or an EWSR1-CREB1 fusion gene, the method comprises the steps of: (a) preparing an expression vector described herein; (b) combining the expression vector with a therapeutic agent carrier; and (c) administering a therapeutically effective amount of the expression vector and therapeutic agent carrier complex to a subject in need thereof.

In some embodiments of this aspect, the therapeutic agent carrier is a compacted DNA nanoparticle or a liposome.

In another aspect, the disclosure provides a method to inhibit an expression of at least one EWSR1-ATF1 fusion gene and/or an EWSR1-CREB1 fusion gene in one or more target cells comprising the steps of: (a) selecting the one or more target cells; and (b) transfecting the target cell with an expression vector described herein, or a composition comprising a therapeutic agent carrier and an expression vector described herein.

In some embodiments of this aspect, the target cells are tumor cells. In some embodiments, the tumor cells are clear cell sarcoma tumor cells.

In another aspect, the disclosure provides a method of suppressing a tumor cell growth in a subject comprising the steps of: (a) identifying the subject in need for suppression of the tumor cell growth; and (b) administering an expression vector described herein or a composition comprising a therapeutic agent carrier and an expression vector described herein, wherein the inhibition results in an apoptosis, an arrested proliferation, or a reduced invasiveness of the tumor cells.

In some embodiments of this aspect, step (a) comprises obtaining a biopsy sample of the tumor cells from the subject. In some embodiments, step (a) comprises sequencing the tumor cells in the biopsy sample to obtain the sequence of an EWSR1-ATF1 fusion gene and/or an EWSR1-CREB1fusion gene.

In some embodiments of this aspect, the method further comprises between step (a) and step (b), a step of providing an expression vector comprising a bifunctional short hairpin RNA (bi-shRNA) sequence specific for knockdown of the EWSR1-ATF1 fusion gene and/or the EWSR1-CREB1 fusion gene, in which the bi-shRNA sequence encodes a nucleic acid sequence capable of hybridizing to a target region of an mRNA transcript encoding the EWSR1-ATF1 fusion gene and/or a target region of an mRNA transcript encoding the EWSR1-CREB1 fusion gene to inhibit the expression of the EWSR1-ATF1 fusion gene and/or the EWSR1-CREB1 fusion gene via RNA interference, and in which the bi-shRNA comprises a first stem-loop structure that comprises an siRNA component and a second stem-loop structure that comprises a miRNA component.

In some embodiments, the tumor cells are clear cell sarcoma tumor cells. In some embodiments, the method treats a cancer in the subject. In certain embodiments, the cancer is clear cell sarcoma. In certain embodiments, the method reduces the size of the tumor. In certain embodiments, the size of the tumor is reduced by at least about 10% (e.g., at least about 10%, 20%, 30%, 40%, 50%, 60%, 70%, 80%, or 90%) relative to the size of the tumor prior to the administering step.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: Exons of EWSR1 mRNA sequence map (RefSeq: NM_005243.4).

FIG. 2: Exons of ATF1 mRNA sequence map (RefSeq: NM 005171.5).

FIG. 3: Sequences (SEQ ID NOs: 57 and 58) and locations of primers to amplify rare fusion protein and endogenous EWSR1.

FIGS. 4A and 4B: Expression comparisons of RT-PCR results from rare fusion amplification and wild-type EWSR1 amplification.

FIG. 5: Triple knockdown joining region sequences (SEQ ID NOs: 59-61) and their targeting sequences.

FIG. 6: The design of triple knockdown constructs in the scaffold of miR17-92a cluster of 6 miRNAs (SEQ ID NOs: 69-74).

FIGS. 7A and 7B: Triple bi-shRNA constructs were designed and constructed with two miR17-92 scaffold formats. FIG. 7A shows the Triple #1 miR17-92 scaffold (SEQ ID NO: 75). FIG. 7B shows the Triple #1 miR17-92 scaffold (SEQ ID NO: 76).

FIGS. 8A, 8B, and 8C: Comparison of target sequences in the pGBI-2011 and pGBI-2013 triple knockdown constructs. FIG. 8A shows the pGBI-2011 triple knockdown construct. SEQ ID NO: 77 is indicated. FIG. 8B shows the pGBI-2013 triple knockdown construct. SEQ ID NO: 78 is indicated. FIG. 8C shows EWSR1-ATF1 fusion type 1 target sequences SEQ ID NOS: 77-80.

FIG. 9: In vitro growth inhibition of Type 1 Cell SU-CCS-1 with triple knockdown plasmids.

FIG. 10: SU-CCS-1 xenograft tumor response in a SU-CCS-1 mouse xenograft model. Arrows at the x-axis indicate treatment with empty lipid nanoparticle (carrier control) or knockdown plasmid.

FIG. 11: SU-CCS-1 xenograft tumor response with VABs™. Arrows at the x-axis indicate treatment days when empty lipid nanoparticle (carrier control) or knockdown plasmid were administered.

FIG. 12: mRNA levels from RT-PCR. Lanes 1-3 contain mRNA from tumors treated with pGBI-2013. Lanes 4-6 contain mRNA from tumors treated with empty liposome. Lane 7 contains mouse RNA control. Fusion=mRNA of EWSR1 Exon 11-ATF1 Exon 6 Type 1 fusion. TUBB=mRNA of β-Tubulin.

### DETAILED DESCRIPTION

Disclosed herein, in certain embodiments, are expression vectors comprising a bifunctional short hairpin RNA (bi-shRNA) sequence specific for knockdown of an EWSR1-ATF1 fusion gene and/or an EWSR1-CREB1 fusion gene. The bi-shRNA sequence encodes a nucleic acid sequence capable of hybridizing to one or more regions of an mRNA transcript encoding the EWSR1-ATF1 fusion gene and/or an mRNA transcript encoding the EWSR1-CREB1 fusion gene to inhibit the expression of the EWSR1-ATF1 fusion gene and/or the EWSR1-CREB1 fusion gene via RNA interference. The bi-shRNA comprises a first stem-loop structure that comprises an siRNA component and a second stem-loop structure that comprises a miRNA component. In some embodiments, the bi-shRNA construct can knockdown two or more EWSR1-ATF1 fusion genes. In some embodiments, the bi-shRNA construct can knockdown an EWSR1-ATF1 fusion gene and an EWSR1-CREB1 fusion gene.

The disclosure also provides compositions that comprise an expression vector having a bi-shRNA sequence specific for knockdown of an EWSR1-ATF1 fusion gene and/or an EWSR1-CREB1 fusion gene.

As described in detail further here, the bi-shRNA construct can be provided in one or more liposomes or lipid nanoparticles. In certain embodiments, the liposome or the lipid nanoparticle can comprise one or more agents capable of recognizing and binding to a target cell or a component thereof, such that the bi-shRNA construct can be targeted to a region of a patient in need.

The expression vectors and compositions described herein can be used in methods for inhibiting an expression of at least one EWSR1-ATF1 fusion gene and/or an EWSR1-CREB1 fusion gene in one or more target cells. The expression vectors and compositions described herein can also be used in methods for suppressing a tumor cell growth (e.g., clear cell and related sarcoma tumor cell growth) in a subject.

### Introduction

The EWSR1 gene located on chromosome 22q12 is a member of the TET family, a highly conserved group of multifunctional, RNA-binding proteins. The mRNA transcript of the EWSR1 gene is spliced together with sequences from 17 exons and encodes a 656 amino acid protein that is highly conserved among TET family genes. The protein includes an N-terminal transcriptional activation domain and a C-terminal RNA-binding domain. The EWSR1 gene is involved in various cellular processes, including gene expression, cell signaling, and RNA processing and transport.

More than 90% of clear cell sarcomas are genetically characterized by a recurrent translocation involving the EWSR1 gene. The most common EWSR1 translocation partner for clear cell sarcoma is the activating transcription factor-1 (ATF1) gene on chromosome 12q13. The ATF1 gene encodes a transcription activating factor, which belongs to the ATF subfamily and basic-region leucine zipper (bZIP) family. The ATF1 gene influences cellular physiological processes by regulating the expression of numerous genes that are related to growth, survival, and other cellular activities. The mRNA transcript of ATF1 gene is spliced together with sequences from 7 exons.

Translocation of 5′ end of EWSR1 exons to 3′ end of ATF1 creates a fusion protein joining the amino terminus transcriptional activation domain of EWSR1 to the carboxyl terminus DNA binding domain of the transcription factor ATF1 to aberrantly hyperactivate and dysregulate numerous genes involved in growth, survival, and many cellular activities.

Wang et al. used both reverse transcription polymerase chain reaction (RT-PCR) and EWSR1 fluorescence in situ hybridization (FISH) on formalin-fixed paraffin embedded tissue as an ancillary diagnostic tool for clear cell sarcoma and to analyze the chimeric types and variants. In the publication, four types of EWSR1-ATF1 fusions were identified, sequencing of chimeric transcripts identified Type 1 (EWSR1 exon 8/ATF1 exon 4), Type 2 (EWSR1 exon 7/ATF1 exon 5), Type 3 (EWSR1 exon 10/ATF1 exon 5), and Type 4 (EWSR1 exon 7/ATF1 exon 7), with occurrence frequency observed at 50%, 45%, >5%, and >1%, respectively. More recently, a novel clear cell sarcoma translocation was identified between EWSR1 exon 11 and ATF1 exon 6. As identified clonal driver mutation, EWSR1-ATF1 translocations are the key genetic factors for oncogenesis of a subset of clear cell sarcoma.

In some cases, the translocation involving the EWSR1 gene results in the formation of a gene fusion between the EWSR1 locus and an ETS transcription factor gene FLI1 t(11;22)(q24;q12). The rearranged fusion genes can occur between different exons resulting in at least four types (1-4) of mRNAs. In some cases, the translocation involves the EWSR1 locus and an ETS transcription factor gene ERG t(21;22)(q22;q12), which produces a EWSR1-ERG fusion. Further non-limiting examples of genes found in chromosomal rearrangements or translocations with EWSR1 include CREB3L1/2; DDIT3; POU5F1; PBX1; PBX3; ZNF444; CREB1; YY1; WT1; NR4A3; ETS family genes ERG, FEV, ETV1, ETV4, and ZSG; and non-ETS family genes NFATC2, PATZ1, SP3, and SMARCA5. EWSR1 fusion variants are discussed in detail, for example, in Jo, Vickie Y. Cancer Cytopathology vol. 128, 4 (2020): 229-231.

There are no current therapies that directly target EWSR1 fusion proteins. Disclosed here is a novel therapeutic approach that utilizes a bi-functional short hairpin RNA interference strategy to directly target Type 1, Type 2, and a rare EWSR1/ATF1 fusion transcript.

A second less common translocation partner of EWSR1 is CREB1 located on chromosome 2q34, which results in a rearrangement t(2;22) that has been preferentially reported in clear cell sarcomas of the gastrointestinal tract and not in clear cell sarcomas of the soft tissue, until recently (Hisaoka et al., Am J Surg Pathol 2008; 32:452-460). Only one EWSR1-CREB1 fusion type (EWSR1 exon 7/CREB1 exon 7) has been described so far.

The disclosure provides expression vectors and compositions comprising thereof that can be used to knock down at least one EWSR1-ATF1 fusion gene and/or an EWSR1-CREB1 fusion gene.

### Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs sets, each acting against the expression of two different genes but in the “traditional” siRNA mode.

As used herein, the term “EWSR1-ATF1 fusion” refers to a gene or a protein that is a product of a translocation involving the EWSR1 gene and the ATF1 gene. The translocation product creates a fusion protein joining the amino terminus transcriptional activation domain portion of EWSR1 to the carboxyl terminus DNA binding domain portion of the transcription factor ATF1. The fusion proteins have EWSR1, or a portion thereof, at the N-terminus and ATF1, or a portion thereof, at the C-terminus. Such translocation events can aberrantly hyperactivate and dysregulate numerous genes involved in growth, survival, and many cellular activities. Examples of EWSR1-ATF1 fusion genes include, but are not limited to, Type 1 (EWSR1 exon 8/ATF1 exon 4; EWSR1 5′ end sequence up to exon 8 fused with ATF1 from exon 4 to 3′ end sequence of ATF1), Type 2 (EWSR1 exon 7/ATF1 exon 5; EWSR1 5′ end sequence up to exon 7 fused with ATF1 from exon 5 to 3′ end sequence of ATF1), Type 3 (EWSR1 exon 10/ATF1 exon 5; EWSR1 5′ end sequence up to exon 10 fused with ATF1 from exon 5 to 3′ end sequence of ATF1), Type 4 (EWSR1 exon 7/ATF1 exon 7; EWSR1 5′ end sequence up to exon 7 fused with ATF1 from exon 7 to 3′ end sequence of ATF1), Type 5 (EWSR1 exon 7/ATF1 exon 4; EWSR1 5′ end sequence up to exon 7 fused with ATF1 from exon 4 to 3′ end sequence of ATF1), Type 6 (EWSR1 exon 9/ATF1 exon 4; EWSR1 5′ end sequence up to exon 9 fused with ATF1 from exon 4 to 3′ end sequence of ATF1), and Type 7 (EWSR1 exon 7/ATF1 exon 6; EWSR1 5′ end sequence up to exon 7 fused with ATF1 from exon 6 to 3′ end sequence of ATF1) (see, e.g., Tsukamoto et al., Pathology—Research and Practice 209 (2013) 803-807). EWSR1-ATF1 fusion genes can be highly patient and tumor specific. The disclosure provides an in-frame fusion gene between the 3′ end of EWSR1 exon 11 fused with the 5′ end of ATF1 exon 6 discovered in a patient's clear cell sarcoma tissue. In some embodiments, the EWSR1-ATF1 fusion gene has the EWSR1 gene or a portion thereof at the 5′ end of the fusion gene and the ATF1 gene or a portion thereof at the 3′ end of the fusion gene. In some embodiments, the fusion gene is an in-frame fusion. In other embodiments, the fusion gene is an off-frame fusion.

As used herein, the term “EWSR1-CREB1 fusion” refers to a gene or a protein that is a product of a translocation involving the EWSR1 gene and the CREB1 gene. The translocation product creates a fusion protein joining the amino terminus transcriptional activation domain portion of EWSR1 to the carboxyl terminus DNA binding domain portion of the transcription factor CREB1. The fusion proteins have EWSR1, or a portion thereof, at the N-terminus and CREB1, or a portion thereof, at the C-terminus. Such translocation events can aberrantly hyperactivate and dysregulate numerous genes involved in growth, survival, and many cellular activities. EWSR1-CREB1 fusion genes are often found in clear cell sarcoma and can be highly patient and tumor specific. In some embodiments, the EWSR1-CREB1 fusion gene has the EWSR1 gene or a portion thereof at the 5′ end of the fusion gene and the CREB1 gene or a portion thereof at the 3′ end of the fusion gene. Currently there is only one known EWSR1-CREB1 fusion protein, which has EWSR1 5′ end sequence up to exon 7 fused with CREB1 from exon 7 to 3′ end sequence of CREB1 (EWSR1 exon 7/CREB1 exon 7). In some embodiments, the fusion gene is an in-frame fusion. In other embodiments, the fusion gene is an off-frame fusion.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the autologous tumor cell vaccine required to provide a clinically significant decrease in disease symptoms without undue adverse side effects. In another example, an “effective amount” for therapeutic uses is the amount of the autologous tumor cell vaccine as disclosed herein required to prevent a recurrence of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the autologous tumor cell vaccine, age, weight, general condition of the subject, the condition being treated, the severity of the condition being treated, and the judgment of the prescribing physician. The phrase “therapeutically effective amount” of a bi-shRNA refers to a sufficient amount of the sequence to knockdown the expression of an EWSR1-ATF1 fusion gene and/or an EWSR1-CREB1 fusion gene in the targeted cells of the patient and/or to otherwise suppress tumor cell growth and/or to treat cancer in the patient. It will be understood, however, that the bi-shRNA sequence and related compositions will be decided by the attending physician, within the scope of sound medical judgment.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing recurrence or prophylactically treating recurrence of the disease or condition, or inhibiting the symptoms of the disease or condition either prophylactically and/or therapeutically. In a non-limiting example, for prophylactic benefit, an autologous tumor cell vaccine composition disclosed herein is administered to an individual at risk of developing a particular disease or condition, predisposed to developing a particular disease or condition, or to an individual previously suffering from and treated for the disease or condition. In some embodiments, the disease or condition is cancer, such as clear cell sarcoma, lymphoma, and leukemia (e.g., clear cell sarcoma).

As used herein, the term “transfection” refers to the introduction of foreign DNA into eukaryotic cells. In some embodiments, transfection is accomplished by any suitable means, such as for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

### Therapeutic Carriers

Therapeutic carriers can be delivery vehicles and/or pharmaceutically acceptable carriers that can be used to deliver the bi-shRNA construct described herein, or expression vectors and compositions comprising thereof, to the tissue or cell of interest. In some embodiments, the therapeutic carriers can be liposomes or lipid nanoparticles. The use of lipids, liposomes, exosomes, proteins or other particle forming compositions as a delivery vehicle for the bi-shRNA constructs is described herein. Liposomes are attractive carriers insofar as they protect biological molecules, such as the bi-shRNA sequences described herein, from degradation while improving cellular uptake. One of the most commonly used classes of liposome formulations for delivering polyanions (e.g., DNA) is that which contains cationic lipids. Other delivery systems, e.g., viral and non-viral delivery systems (e.g., cationic polymers, poly(L-lysine), polysaccharides, and poly(ethylenimine)s) may also be considered and used (see, e.g., Sung and Kim, Biomater Res. 2019 Mar. 12:23:8).

In some embodiments, the liposome is comprised of 1,2-dioleoyl-3-trimethylammonium-propane (DOTAP) and cholesterol (also referred to as “DOTAP:cholesterol”). DOTAP:cholesterol liposomes has been used to deliver nucleic acid payloads in vivo and in clinical trials. The DOTAP:cholesterol liposome fuses with the cell membrane and enters the cell via endosomes, after which the nucleic acid payloads are released into the cytosol. The benefits of DOTAP:cholesterol liposomes as a delivery system include their ability to overcome the interstitial pressure gradient and penetrate tight barriers throughout a target organ, non-toxicity, non-immunogenicity, enhanced stability, and the ability to biodegrade. The DOTAP:cholesterol delivery system is discussed in detail, e.g., in Butt et al., Genes (Basel) 2022.

DOTAP and cholesterol can be combined at various ratios to produce liposomes of various sizes and/or to modulate transfection efficiency. For example, the amount of cholesterol used can influence the curvature of the lipid bilayer, thereby altering the size of liposomes. In some embodiments, the molar ratio of DOTAP:cholesterol is about 1:1, 1:2, 1:3, 1:4, 1:5, 1:6, 1:7, 1:8, 1:9, or 1:10.

In some embodiments, the DOTAP:cholesterol formulation is comprised of DOTAP and cholesterol dissolved ethanol. In some embodiments, the concentration of DOTAP in the formulation is 1-50 mmol/L, 1-40 mmol/L, 1-30 mmol/L, 1-20 mmol/L, 5-20 mmol/L, 8-20 mmol/L, 8-12 mmol/L, about 5 mmol/L, about 6 mmol/L, about 7 mmol/L, about 8 mmol/L, about 9 mmol/L, about 10 mmol/L, about 11 mmol/L, about 12 mmol/L, about 13 mmol/L, about 14 mmol/L, or about 15 mmol/L. In some embodiments, the concentration of cholesterol in the formulation is 1-50 mmol/L, 1-40 mmol/L, 1-30 mmol/L, 1-20 mmol/L, 5-20 mmol/L, 8-20 mmol/L, 8-12 mmol/L, about 5 mmol/L, about 6 mmol/L, about 7 mmol/L, about 8 mmol/L, about 9 mmol/L, about 10 mmol/L, about 11 mmol/L, about 12 mmol/L, about 13 mmol/L, about 14 mmol/L, or about 15 mmol/L.

In some embodiments, the DOTAP:cholesterol formulation can include an aqueous solutions, for example, dextrose dissolved in water (“D5W”). In some embodiments, the concentration of D5W is 1-20%, 1-10%, 1-5%, 3-10%, about 1%, about 2%, about 3%, about 4%, about 5%, about 6%, about 7%, about 8%, about 9%, or about 10% D5W). In some embodiments, the final ethanol concentration in the DOTAP:cholesterol formulation is less than 5%, 4%, 3%, 2%, 1%, 0.9%, 0.8%, 0.7%, 0.6%, 0.5%, 0.4%, 0.3%, 0.2%, or 0.1%. Preparation of DOTAP:cholesterol formulations are described in Rao, D. D., et al., Mol Ther, 2016. 24(8): p. 1412-22.

Lipid aggregates may be formed with macromolecules using cationic lipids alone or including other lipids and amphiphiles, such as phosphatidylethanolamine. It is well-known in the art that both the composition of the lipid formulation, as well as its method of preparation, have an effect on the structure and size of the resultant anionic macromolecule-cationic lipid aggregate. These factors can be modulated to optimize delivery of polyanions to specific cell types in vitro and in vivo.

The use of cationic lipids for cellular delivery of the bi-shRNA sequences described herein has several advantages. The encapsulation of anionic compositions using cationic lipids is essentially quantitative due to electrostatic interaction. In addition, it is believed that the cationic lipids interact with the negatively charged cell membranes, thereby initiating cellular membrane transport.

Experiments have shown that plasmid DNA may be encapsulated in small particles, which generally consist of a single plasmid encapsulated within a bilayer lipid vesicle (Wheeler, et al., 1999, Gene Therapy 6, 271-281). These particles often contain the fusogenic lipid such as dioleoylphosphatidylethanolamine (DOPE), low levels of a cationic lipid, and can be stabilized in aqueous media by the presence of a poly(ethylene glycol) (PEG) coating.

These lipid particles have systemic applications, as they exhibit extended circulation lifetimes following intravenous (i.v.) injection, can accumulate preferentially in various tissues and organs due to the enhanced vascular permeability in such regions, and can be designed to escape the lyosomic pathway of endocytosis by disruption of endosomal membranes. These properties can be useful in delivering biologically active molecules to various cell types for experimental and therapeutic applications. Various lipid nucleic acid particles and methods of preparation thereof are described in U.S. Patent Application Publication Nos. 2008-0020058, 2003-0077829, 2003-0108886, 2006-0051405, 2006-0083780, 2003-0104044, 2006-0051405, 2004-0142025, 2006-00837880, 2005-0064595, 2005-0175682, 2005-0118253, 2005-0255153 and 2005-0008689; and U.S. Pat. Nos. 5,885,613; 6,586,001; 6,858,225; 6,858,224; 6,815,432; 6,586,410; 6,534,484; and 6,287,591, all of which are incorporated herein by reference in their entirety.

The disclosure provides that the bi-shRNA constructs, and the associated delivery vehicles used therewith, may be targeted towards specific cell types, for example, tumor cells, and the like. For example, the liposomal nanoparticles can be directed to bind to cell surfaces by a number of specific interactions. This binding facilitates the uptake of the DNA into the cell by one of several well understood cell entry pathways. Rapid sequestration of the nanoparticles (e.g., liposomes) by these interactions reduces their time in the peripheral circulation, thereby decreasing the likelihood of degradation and nonspecific uptake. General targeting agents include, but are not limited to, transferrin (Trf) which binds to the transferrin receptor (TrfR) on a cell surface-or using an antibody (or a derivative thereof) that binds to the TrfR on the cell surface. Erbitux (an EGFR monoclonal antibody approved for human use) is an exemplary agent for EGFR-targeting, which may also be used to decorate the liposomal nanoparticles described herein. Additional targeting moieties can be, but are not limited to, lectins or small molecules (peptides or carbohydrates) which recognize and bind to specific targets found only on (or are more restricted to) cancer or tumor cells. In some embodiments, specific targets on tumor cells can be tumor specific neoantigens. In some embodiments, a specific target can be CD99, which is found on sarcoma tumor cells. The advantage of smaller (and possibly higher affinity) molecules is that they could be present at a higher density on the surface of the nanoparticles employed.

In one aspect, the particle comprises a Doggybone (dbDNA™) DNA. Named after its schematic structure, dbDNA™ is a minimal, linear, double stranded and covalently closed DNA construct. The Doggybone platform is available from the commercial supplier Touchlight.

### Administration

The bi-shRNA constructs described herein, which can be delivered to a system in connection with an appropriate therapeutic carrier (such as a liposome or lipid nanoparticle), may be administered to a system using any of various well-known techniques. For example, in the case of a mammal, the bi-shRNA sequence may be administered to a mammal via parenteral injection. The term “parenteral,” as used herein, includes subcutaneous, intracutaneous, intravenous, intramuscular, intraarticular, intraarterial, intrasynovial, or infusion techniques. In particular embodiments, the bi-shRNA sequence can be administered via intramuscular injection. In some embodiments, the bi-shRNA sequence can be administered via systemic infusion. In particular embodiments, the systemic infusion delivers the bi-shRNA sequence into tumor cells or tissues.

The bi-shRNA sequence, and related compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated composition or its delivery form. For example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation ofinjectables.

According to certain embodiments, a Plasma-Lyte® carrier may be employed and used to deliver the bi-shRNA sequence, particularly for parenteral injection (e.g., intramuscular injection). Plasma-Lyte® is a sterile, non-pyrogenic isotonic solution that may be used for intravenous administration. Each 100 mL volume contains 526 mg of Sodium Chloride, USP (NaCl); 502 mg of Sodium Gluconate (C6H11NaO7); 368 mg of Sodium Acetate Trihydrate, USP (C2H3NaO2.3H2O); 37 mg of Potassium Chloride, USP (KCl); and 30 mg of Magnesium Chloride, USP (MgCl2.6H2O). It contains no antimicrobial agents. The pH is preferably adjusted with sodium hydroxide to about 7.4 (6.5 to 8.0).

The injectable formulations used to deliver the bi-shRNA sequence may be sterilized, for example, by filtration through a bacterial-retaining filter, or by incorporating sterilizing agents in the form of sterile solid compositions, which can be dissolved or dispersed in sterile water, Plasma-Lyte® or other sterile injectable medium prior to use.

In order to prolong the expression of the bi-shRNA sequence within a system (or to prolong the effect thereof), it may be desirable to slow the absorption of the composition from subcutaneous or intramuscular injection. This may be accomplished by the use of a liquid suspension of crystalline or amorphous material with poor water solubility. The rate of absorption of the composition may then depend upon its rate of dissolution, which, in turn, may depend upon crystal size and crystalline form.

Alternatively, delayed absorption of a parenterally administered bi-shRNA sequence may be accomplished by dissolving or suspending the composition in an oil vehicle. Injectable depot forms may be prepared by forming microencapsule matrices of the bi-shRNA sequence in biodegradable polymers such as polylactide-polyglycolide. Depending upon the ratio of the bi-shRNA sequence material to polymer and the nature of the particular polymer employed, the rate of the bi-shRNA sequence release can be controlled. Examples of other biodegradable polymers include poly(orthoesters) and poly(anhydrides). As described above, depot injectable formulations may also be prepared by entrapping the bi-shRNA sequence in liposomes (or even microemulsions) that are compatible with the target body tissues, such as muscular tissue.

According to additional related embodiments of the present invention, methods for suppressing tumor growth and/or treating cancer (e.g., clear cell sarcoma) are provided. Such methods generally comprise providing a patient with therapeutically effective amounts of a bi-shRNA sequence that knocks down the expression of at least one EWSR1-ATF1 fusion gene and/or an EWSR1-CREB1 fusion gene in the patient by administration at a desired location in the patient, e.g., location of cancer, cancerous cells or tissues. In certain embodiments, the bi-shRNA sequence may, preferably, be delivered to a patient in connection with a lipid nanoparticle and a carrier similar to that of Plasma-Lyte®, via parenteral injection.

The specific therapeutically effective dose level for any particular patient may depend upon a variety of factors, including the severity of a patient's disorder; the activity of the specific EWSR1-ATF1 fusion gene and/or EWSR1-CREB1 fusion gene and the bi-shRNA sequence employed; the delivery vehicle employed; the age, body weight, general health, gender and diet of the patient; the time of administration, route of administration, the rate/speed of administration and rate of excretion of the specific bi-shRNA sequence employed; the duration of the treatment; drugs used in combination or contemporaneously with the specific bi-shRNA sequence employed; and like factors well-known in the medical arts.

Upon improvement of a patient's condition, a maintenance dose of a bi-shRNA sequence may be administered, if necessary. Subsequently, the dosage or frequency of administration, or both, may be reduced, as a function of the symptoms, to a level at which the improved condition is retained when the symptoms have been alleviated to the desired level.

According to yet further embodiments of the invention, novel compositions are provided for expressing the bi-shRNA in a system. As described herein, the bi-shRNA sequence may comprise various transcriptional control elements, such as a promoter, termination sequence, and others. Also as described relative to other embodiments of the present invention, the bi-shRNA sequence may be disposed within or connected to an appropriate vehicle for delivery to a system, such as a liposome or lipid nanoparticle. Still further, according to such embodiments, the delivery vehicle may, optionally, be decorated with agents that are capable of recognizing and binding to target cells or tissues, such as tumor cells (e.g., clear cell sarcoma tumor cells).

### Expression Vector

Expression vectors of the present disclosure contain a bi-shRNA that is capable of reducing (“knocking down”) the expression of an EWSR1 fusion gene disclosed herein. In some embodiments, the bi-shRNA that knocks down the EWSR1-ATF1 fusion gene and/or the EWSR1-CREB1 fusion gene. In general, the bi-shRNA comprises a first stem-loop structure that comprises an siRNA component and a second stem-loop structure that comprises a miRNA component. The miRNA component comprises a seed region, which is a short sequence of about 2 to 8 or more nucleotides that is located at the 5′ end of the miRNA. The seed region is important for miRNA binding to a target mRNA and the sequence of the seed region is a determinant of target specificity. In some embodiments, the sequence that targets the fusion junction of EWSR1-ATF1 or EWSR1-CREB1 is placed within the seed region of the miRNA portion of the bi-shRNA. In some embodiments, the bifunctional shRNA (bi-shRNA) has two mechanistic pathways of action, that of the siRNA and that of the miRNA. Thus, in some embodiments, the bifunctional shRNA described herein is different from a traditional shRNA, i.e., a DNA transcription derived RNA acting by the siRNA mechanism of action or from a “doublet or more shRNA” that refers to two or more shRNAs, each acting against the expression of two or more different genes but in the traditional siRNA mode. In some embodiments, the bi-shRNA incorporates siRNA (cleavage dependent) and miRNA (cleavage-independent) motifs.

Bifunctional shRNA

An expression vector can comprise a bi-shRNA sequence specific for knockdown of one or more EWSR1-ATF1 fusion genes and/or an EWSR1-CREB1 fusion gene. In some embodiments, the bifunctional shRNA sequence encodes a nucleic acid sequence capable of hybridizing to one or more regions of an mRNA transcript encoding the EWSR1-ATF1 fusion gene and/or an mRNA transcript encoding the EWSR1-CREB1 fusion gene to inhibit the expression of the EWSR1-ATF1 fusion gene and/or the EWSR1-CREB1 fusion gene via RNA interference. In some embodiments, the bi-shRNA sequence targets at the joining sequence of the EWSR1-ATF1 fusion gene transcript between EWSR1 and ATF1. In some embodiments, the bi-shRNA sequence targets at the joining sequence of the EWSR1-CREB1 fusion gene transcript between EWSR1 and CREB1.

In some embodiments, due to sequence similarity of the joining sequences in multiple EWSR1-ATF1 or EWSR1-CREB1 fusion genes, a single bi-shRNA sequence can be designed to specifically knockdown two or more different EWSR1-ATF1 fusion genes (e.g., two, three, four, five, six, seven, eight, nine, or ten different EWSR1-ATF1 fusion genes) at the same time.

In some embodiments, the bi-shRNA can knockdown two EWSR1-ATF1 fusion genes at the same time. For example, the two EWSR1-ATF1 fusion genes can be any one of the following pairs in the brackets: [Type 1, Type 2]; [Type 1, Type 3]; [Type 1, Type 4]; [Type 1, Type 5]; [Type 1, Type 6]; [Type 1, Type 7]; [Type 2, Type 3]; [Type 2, Type 4]; [Type 2, Type 5]; [Type 2, Type 6]; [Type 2, Type 7]; [Type 3, Type 4]; [Type 3, Type 5]; [Type 3, Type 6]; [Type 3, Type 7]; [Type 4, Type 5]; [Type 4, Type 6]; [Type 4, Type 7]; [Type 5, Type 6]; [Type 5, Type 7]; and [Type 6, Type 7].

In some embodiments, the bi-shRNA can knockdown three EWSR1-ATF1 fusion genes at the same time. For example, the three EWSR1-ATF1 fusion genes can be any one of the following triplets in the brackets: [Type 1, Type 2, Type 3]; [Type 1, Type 2, Type 4]; [Type 1, Type 2, Type 5]; [Type 1, Type 2, Type 6]; [Type 1, Type 2, Type 7]; [Type 1, Type 3, Type 4]; [Type 1, Type 3, Type 5]; [Type 1, Type 3, Type 6]; [Type 1, Type 3, Type 7]; [Type 1, Type 4, Type 5]; [Type 1, Type 4, Type 6]; [Type 1, Type 4, Type 7]; [Type 1, Type 5, Type 6]; [Type 1, Type 5, Type 7]; [Type 1, Type 6, Type 7]; [Type 2, Type 3, Type 4]; [Type 2, Type 3, Type 5]; [Type 2, Type 3, Type 6]; [Type 2, Type 3, Type 7]; [Type 2, Type 4, Type 5]; [Type 2, Type 4, Type 6]; [Type 2, Type 4, Type 7]; [Type 2, Type 5, Type 6]; [Type 2, Type 5, Type 7]; [Type 2, Type 6, Type 7]; [Type 3, Type 4, Type 5]; [Type 3, Type 4, Type 6]; [Type 3, Type 4, Type 7]; [Type 3, Type 5, Type 6]; [Type 3, Type 5, Type 7]; [Type 3, Type 6, Type 7]; [Type 4, Type 5, Type 6]; [Type 4, Type 5, Type 7]; [Type 4, Type 6, Type 7]; and [Type 5, Type 6, Type 7].

In some embodiments, the bi-shRNA can knockdown four EWSR1-ATF1 fusion genes at the same time. For example, the four EWSR1-ATF1 fusion genes can be any one of the following sets in the brackets: [Type 1, Type 2, Type 3, Type 4]; [Type 1, Type 2, Type 3, Type 5]; [Type 1, Type 2, Type 3, Type 6]; [Type 1, Type 2, Type 3, Type 7]; [Type 1, Type 2, Type 4, Type 5]; [Type 1, Type 2, Type 4, Type 6]; [Type 1, Type 2, Type 4, Type 7]; [Type 1, Type 2, Type 5, Type 6]; [Type 1, Type 2, Type 5, Type 7]; [Type 1, Type 2, Type 6, Type 7]; [Type 1, Type 3, Type 4, Type 5]; [Type 1, Type 3, Type 4, Type 6]; [Type 1, Type 3, Type 4, Type 7]; [Type 1, Type 3, Type 5, Type 6]; [Type 1, Type 3, Type 5, Type 7]; [Type 1, Type 3, Type 6, Type 7]; [Type 1, Type 4, Type 5, Type 6]; [Type 1, Type 4, Type 5, Type 7]; [Type 1, Type 4, Type 6, Type 7]; [Type 1, Type 5, Type 6, Type 7]; [Type 2, Type 3, Type 4, Type 5]; [Type 2, Type 3, Type 4, Type 6]; [Type 2, Type 3, Type 4, Type 7]; [Type 2, Type 3, Type 5, Type 6]; [Type 2, Type 3, Type 5, Type 7]; [Type 2, Type 3, Type 6, Type 7]; [Type 2, Type 4, Type 5, Type 6]; [Type 2, Type 4, Type 5, Type 7]; [Type 2, Type 4, Type 6, Type 7]; [Type 2, Type 5, Type 6, Type 7]; [Type 3, Type 4, Type 5, Type 6]; [Type 3, Type 4, Type 5, Type 7]; [Type 3, Type 4, Type 6, Type 7]; [Type 3, Type 5, Type 6, Type 7]; and [Type 4, Type 5, Type 6, Type 7].

In some embodiments, the bi-shRNA can knockdown five EWSR1-ATF1 fusion genes at the same time. For example, the five EWSR1-ATF1 fusion genes can be any one of the following sets in the brackets: [Type 1, Type 2, Type 3, Type 4, Type 5]; [Type 1, Type 2, Type 3, Type 4, Type 6]; [Type 1, Type 2, Type 3, Type 4, Type 7]; [Type 1, Type 2, Type 3, Type 5, Type 6]; [Type 1, Type 2, Type 3, Type 5, Type 7]; [Type 1, Type 2, Type 3, Type 6, Type 7]; [Type 1, Type 2, Type 4, Type 5, Type 6]; [Type 1, Type 2, Type 4, Type 5, Type 7]; [Type 1, Type 2, Type 4, Type 6, Type 7]; [Type 1, Type 2, Type 5, Type 6, Type 7]; [Type 1, Type 3, Type 4, Type 5, Type 6]; [Type 1, Type 3, Type 4, Type 5, Type 7]; [Type 1, Type 3, Type 4, Type 6, Type 7]; [Type 1, Type 3, Type 5, Type 6, Type 7]; [Type 1, Type 4, Type 5, Type 6, Type 7]; [Type 2, Type 3, Type 4, Type 5, Type 6]; [Type 2, Type 3, Type 4, Type 5, Type 7]; [Type 2, Type 3, Type 4, Type 6, Type 7]; [Type 2, Type 3, Type 5, Type 6, Type 7]; [Type 2, Type 4, Type 5, Type 6, Type 7]; and [Type 3, Type 4, Type 5, Type 6, Type 7].

In some embodiments, the bi-shRNA can knockdown six EWSR1-ATF1 fusion genes at the same time. For example, the six EWSR1-ATF1 fusion genes can be any one of the following sets in the brackets: [Type 1, Type 2, Type 3, Type 4, Type 5, Type 6]; [Type 1, Type 2, Type 3, Type 4, Type 5, Type 7]; [Type 1, Type 2, Type 3, Type 4, Type 6, Type 7]; [Type 1, Type 2, Type 3, Type 5, Type 6, Type 7]; [Type 1, Type 2, Type 4, Type 5, Type 6, Type 7]; [Type 1, Type 3, Type 4, Type 5, Type 6, Type 7]; and [Type 2, Type 3, Type 4, Type 5, Type 6, Type 7].

In some embodiments, the bi-shRNA can knockdown seven EWSR1-ATF1 fusion genes at the same time (e.g., Type 1, Type 2, Type 3, Type 4, Type 5, Type 6, and Type 7 EWSR1-ATF1 fusion genes).

### Single Knockdown of EWSR1-ATF1 Fusion Gene

In some embodiments, a bi-shRNA sequence can knockdown the expression of a Type 1 EWSR1-ATF1 fusion gene that comprises EWSR1 5′ end sequence up to exon 8 fused with ATF1 from exon 4 to 3′ end sequence of ATF1. In some embodiments, a bi-shRNA sequence can knockdown the expression of a Type 2 EWSR1-ATF1 fusion gene that comprises EWSR1 5′ end sequence up to exon 7 fused with ATF1 from exon 5 to 3′ end sequence of ATF1. In some embodiments, a bi-shRNA sequence can knockdown the expression of a Type 3 EWSR1-ATF1 fusion gene that comprises EWSR1 5′ end sequence up to exon 10 fused with ATF1 from exon 5 to 3′ end sequence of ATF1. In some embodiments, a bi-shRNA sequence can knockdown the expression of a Type 4 EWSR1-ATF1 fusion gene that comprises EWSR1 5′ end sequence up to exon 7 fused with ATF1 from exon 7 to 3′ end sequence of ATF1.

For the embodiments described above, in some embodiments, a bifunctional shRNA sequence that knocks down a Type 1 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:31. In some embodiments, a bifunctional shRNA sequence that knocks down a Type 2 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:32. In some embodiments, a bifunctional shRNA sequence that knocks down a Type 3 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:33. In some embodiments, a bifunctional shRNA sequence that knocks down a Type 4 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:34.

In some embodiments, a bi-shRNA sequence can knockdown the expression of a patient specific EWSR1-ATF1 fusion gene. A patient specific EWSR1-ATF1 fusion gene can be determined by sequencing the tumor cells in a biopsy sample from the patient. In some embodiments, a bi-shRNA sequence can knockdown the expression of a patient specific EWSR1-ATF1 fusion gene that comprises EWSR1 5′ end sequence up to exon 11 fused with ATF1 from exon 6 to 3′ end sequence of ATF1.

In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:21. In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:22. In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:23. In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:24. In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:25. In some embodiments, a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) can comprise a sequence having at least 90% (e.g., 90%, 91%, 92%, 93%, 94%, 95%, 96%, 97%, 98%, 99%, or 100%) identity to the sequence of SEQ ID NO:26.

### Double Knockdown of EWSR1-ATF1 Fusion Genes

In some embodiments, a bi-shRNA sequence can knockdown the expression of two EWSR1-ATF1 fusion genes. In some embodiments, a bi-shRNA sequence can knockdown the expression of a Type 1 EWSR1-ATF1 fusion gene and a Type 2 EWSR1-ATF1 fusion gene. In some embodiments, a bi-shRNA sequence can knockdown the expression of a Type 1 EWSR1-ATF1 fusion gene and a Type 3 EWSR1-ATF1 fusion gene. In some embodiments, a bi-shRNA sequence can knockdown the expression of a Type 1 EWSR1-ATF1 fusion gene and a Type 4 EWSR1-ATF1 fusion gene. In some embodiments, a bi-shRNA sequence can knockdown the expression of a Type 2 EWSR1-ATF1 fusion gene and a Type 3 EWSR1-ATF1 fusion gene. In some embodiments, a bi-shRNA sequence can knockdown the expression of a Type 2 EWSR1-ATF1 fusion gene and a Type 4 EWSR1-ATF1 fusion gene. In some embodiments, a bi-shRNA sequence can knockdown the expression of a Type 3 EWSR1-ATF1 fusion gene and a Type 4 EWSR1-ATF1 fusion gene.

In some embodiments, a bi-shRNA sequence can knockdown the expression of any one of Type 1, Type 2, Type 3, and Type 4 EWSR1-ATF1 fusion genes and a patient specific EWSR1-ATF1 fusion gene. A patient specific EWSR1-ATF1 fusion gene can be determined by sequencing the tumor cells in a biopsy sample from the patient. A bi-shRNA construct can then be designed that can knockdown the expression of any one of Type 1, Type 2, Type 3, and Type 4 EWSR1-ATF1 fusion genes and the patient specific EWSR1-ATF1 fusion gene. In some embodiments, a bi-shRNA sequence can knockdown the expression of a Type 1 EWSR1-ATF1 fusion gene and a patient specific EWSR1-ATF1 fusion gene. In some embodiments, a bi-shRNA sequence can knockdown the expression of a Type 2 EWSR1-ATF1 fusion gene and a patient specific EWSR1-ATF1 fusion gene. In some embodiments, a bi-shRNA sequence can knockdown the expression of a Type 3 EWSR1-ATF1 fusion gene and a patient specific EWSR1-ATF1 fusion gene. In some embodiments, a bi-shRNA sequence can knockdown the expression of a Type 4 EWSR1-ATF1 fusion gene and a patient specific EWSR1-ATF1 fusion gene.

In other embodiments, to knockdown two different EWSR1-ATF1 fusion genes, two different bi-shRNA constructs can be used. In some embodiments, a first bi-shRNA sequence can knockdown the expression of a Type 1 EWSR1-ATF1 fusion gene and a second bi-shRNA sequence can knockdown the expression of a Type 2 EWSR1-ATF1 fusion gene. In some embodiments, a first bi-shRNA sequence can knockdown the expression of a Type 1 EWSR1-ATF1 fusion gene and a second bi-shRNA sequence can knockdown the expression of a Type 3 EWSR1-ATF1 fusion gene. In some embodiments, a first bi-shRNA sequence can knockdown the expression of a Type 1 EWSR1-ATF1 fusion gene and a second bi-shRNA sequence can knockdown the expression of a Type 4 EWSR1-ATF1 fusion gene. In some embodiments, a first bi-shRNA sequence can knockdown the expression of a Type 2 EWSR1-ATF1 fusion gene and a second bi-shRNA sequence can knockdown the expression of a Type 3 EWSR1-ATF1 fusion gene. In some embodiments, a first bi-shRNA sequence can knockdown the expression of a Type 2 EWSR1-ATF1 fusion gene and a second bi-shRNA sequence can knockdown the expression of a Type 4 EWSR1-ATF1 fusion gene. In some embodiments, a first bi-shRNA sequence can knockdown the expression of a Type 3 EWSR1-ATF1 fusion gene and a second bi-shRNA sequence can knockdown the expression of a Type 4 EWSR1-ATF1 fusion gene.

In some embodiments, a first bi-shRNA sequence can knockdown the expression of any one of Type 1, Type 2, Type 3, and Type 4 EWSR1-ATF1 fusion genes and a second bi-shRNA sequence can knockdown the expression of a patient specific EWSR1-ATF1 fusion gene. In some embodiments, a first bi-shRNA sequence can knockdown the expression of a Type 1 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 2 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 3 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 4 EWSR1-ATF1 fusion gene and a second bi-shRNA sequence can knockdown the expression of a patient specific EWSR1-ATF1 fusion gene.

For any of the embodiments described above, in some embodiments, a bifunctional shRNA sequence that knocks down a Type 1 EWSR1-ATF1 fusion gene and a Type 2 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:35. In some embodiments, a bifunctional shRNA sequence that knocks down a Type 1 EWSR1-ATF1 fusion gene and a Type 3 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:36. In some embodiments, a bifunctional shRNA sequence that knocks down a Type 1 EWSR1-ATF1 fusion gene and a Type 4 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:37. In some embodiments, a bifunctional shRNA sequence that knocks down a Type 2 EWSR1-ATF1 fusion gene and a Type 3 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:38. In some embodiments, a bifunctional shRNA sequence that knocks down a Type 2 EWSR1-ATF1 fusion gene and a Type 4 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:39. In some embodiments, a bifunctional shRNA sequence that knocks down a Type 3 EWSR1-ATF1 fusion gene and a Type 4 EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:40.

For any of the embodiments described above, in some embodiments, a bifunctional shRNA sequence that knocks down a Type 1 EWSR1-ATF1 fusion gene and a patient specific EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:41. In some embodiments, a bifunctional shRNA sequence that knocks down a Type 2 EWSR1-ATF1 fusion gene and a patient specific EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:42. In some embodiments, a bifunctional shRNA sequence that knocks down a Type 3 EWSR1-ATF1 fusion gene and a patient specific EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:43. In some embodiments, a bifunctional shRNA sequence that knocks down a Type 4 EWSR1-ATF1 fusion gene and a patient specific EWSR1-ATF1 fusion gene can comprise a sequence having at least 90% (e.g., 90%, 91%, 92%, 93%, 94%, 95%, 96%, 97%, 98%, 99%, or 100%) identity to the sequence of SEQ ID NO:44.

### Triple Knockdown of EWSR1-ATF1 Fusion Genes

In some embodiments, a bi-shRNA sequence can knockdown the expression of three EWSR1-ATF1 fusion genes, i.e., three EWSR1-ATF1 fusion genes chosen from a Type 1 EWSR1-ATF1 fusion gene, a Type 2 EWSR1-ATF1 fusion gene, a Type 3 EWSR1-ATF1 fusion gene, and a patient specific EWSR1-ATF1 fusion gene. In some embodiments, a bi-shRNA sequence can knockdown the expression of a Type 1 EWSR1-ATF1 fusion gene, a Type 2 EWSR1-ATF1 fusion gene, and a Type 3 EWSR1-ATF1 fusion gene. In some embodiments, a bi-shRNA sequence can knockdown the expression of a Type 1 EWSR1-ATF1 fusion gene, a Type 2 EWSR1-ATF1 fusion gene, and a Type 4 EWSR1-ATF1 fusion gene. In some embodiments, a bi-shRNA sequence can knockdown the expression of a Type 1 EWSR1-ATF1 fusion gene, a Type 3 EWSR1-ATF1 fusion gene, and a Type 4 EWSR1-ATF1 fusion gene. In some embodiments, a bi-shRNA sequence can knockdown the expression of a Type 2 EWSR1-ATF1 fusion gene, a Type 3 EWSR1-ATF1 fusion gene, and a Type 4 EWSR1-ATF1 fusion gene.

In some embodiments, a bi-shRNA sequence can knockdown the expression of a Type 1 EWSR1-ATF1 fusion gene, a Type 2 EWSR1-ATF1 fusion gene, and a patient specific EWSR1-ATF1 fusion gene. In some embodiments, a bi-shRNA sequence can knockdown the expression of a Type 1 EWSR1-ATF1 fusion gene, a Type 3 EWSR1-ATF1 fusion gene, and a patient specific EWSR1-ATF1 fusion gene. In some embodiments, a bi-shRNA sequence can knockdown the expression of a Type 1 EWSR1-ATF1 fusion gene, a Type 4 EWSR1-ATF1 fusion gene, and a patient specific EWSR1-ATF1 fusion gene. In some embodiments, a bi-shRNA sequence can knockdown the expression of a Type 2 EWSR1-ATF1 fusion gene, a Type 3 EWSR1-ATF1 fusion gene, and a patient specific EWSR1-ATF1 fusion gene. In some embodiments, a bi-shRNA sequence can knockdown the expression of a Type 2 EWSR1-ATF1 fusion gene, a Type 4 EWSR1-ATF1 fusion gene, and a patient specific EWSR1-ATF1 fusion gene. In some embodiments, a bi-shRNA sequence can knockdown the expression of a Type 3 EWSR1-ATF1 fusion gene, a Type 4 EWSR1-ATF1 fusion gene, and a patient specific EWSR1-ATF1 fusion gene.

In other embodiments, to knockdown three different EWSR1-ATF1 fusion genes, two or three different bi-shRNA constructs can be used. In some embodiments, two different bi-shRNA constructs can be used. In some embodiments, a first bi-shRNA sequence can knockdown the expression of a Type 1 EWSR1-ATF1 fusion gene and a Type 2 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 1 EWSR1-ATF1 fusion gene and a Type 3 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 1 EWSR1-ATF1 fusion gene and a Type 4 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 2 EWSR1-ATF1 fusion gene and a Type 3 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 2 EWSR1-ATF1 fusion gene and a Type 4 EWSR1-ATF1 fusion gene and a second bi-shRNA sequence can knockdown the expression of a patient specific EWSR1-ATF1 fusion gene. In some embodiments, a first bi-shRNA sequence can knockdown the expression of a Type 3 EWSR1-ATF1 fusion gene and a Type 4 EWSR1-ATF1 fusion gene and a second bi-shRNA sequence can knockdown the expression of a patient specific EWSR1-ATF1 fusion gene.

In some embodiments, three different bi-shRNA constructs can be used. In some embodiments, a first bi-shRNA sequence can knockdown the expression of a Type 1 EWSR1-ATF1 fusion gene, a second bi-shRNA sequence can knockdown the expression of a Type 2 EWSR1-ATF1 fusion gene, and a third bi-shRNA sequence can knockdown the expression of a patient specific EWSR1-ATF1 fusion gene. In some embodiments, a first bi-shRNA sequence can knockdown the expression of a Type 1 EWSR1-ATF1 fusion gene, a second bi-shRNA sequence can knockdown the expression of a Type 3 EWSR1-ATF1 fusion gene, and a third bi-shRNA sequence can knockdown the expression of a patient specific EWSR1-ATF1 fusion gene. In some embodiments, a first bi-shRNA sequence can knockdown the expression of a Type 1 EWSR1-ATF1 fusion gene, a second bi-shRNA sequence can knockdown the expression of a Type 4 EWSR1-ATF1 fusion gene, and a third bi-shRNA sequence can knockdown the expression of a patient specific EWSR1-ATF1 fusion gene. In some embodiments, a first bi-shRNA sequence can knockdown the expression of a Type 2 EWSR1-ATF1 fusion gene, a second bi-shRNA sequence can knockdown the expression of a Type 3 EWSR1-ATF1 fusion gene, and a third bi-shRNA sequence can knockdown the expression of a patient specific EWSR1-ATF1 fusion gene. In some embodiments, a first bi-shRNA sequence can knockdown the expression of a Type 2 EWSR1-ATF1 fusion gene, a second bi-shRNA sequence can knockdown the expression of a Type 4 EWSR1-ATF1 fusion gene, and a third bi-shRNA sequence can knockdown the expression of a patient specific EWSR1-ATF1 fusion gene. In some embodiments, a first bi-shRNA sequence can knockdown the expression of a Type 3 EWSR1-ATF1 fusion gene, a second bi-shRNA sequence can knockdown the expression of a Type 4 EWSR1-ATF1 fusion gene, and a third bi-shRNA sequence can knockdown the expression of a patient specific EWSR1-ATF1 fusion gene.

For any of the embodiments described above, in some embodiments, a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) can comprise a sequence having at least 90% (e.g., 90%, 91%, 92%, 93%, 94%, 95%, 96%, 97%, 98%, 99%, or 100%) identity to the sequence of SEQ ID NO:27. In some embodiments, a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) can comprise a sequence having at least 90% (e.g., 90%, 91%, 92%, 93%, 94%, 95%, 96%, 97%, 98%, 99%, or 100%) identity to the sequence of SEQ ID NO:28. In some embodiments, a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) can comprise a sequence having at least 90% (e.g., 90%, 91%, 92%, 93%, 94%, 95%, 96%, 97%, 98%, 99%, or 100%) identity to the sequence of SEQ ID NO:29. In some embodiments, a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) can comprise a sequence having at least 90% (e.g., 90%, 91%, 92%, 93%, 94%, 95%, 96%, 97%, 98%, 99%, or 100%) identity to the sequence of SEQ ID NO:30.

In some embodiments, a bi-shRNA sequence mentioned above can be in an expression vector, such as an expression vector in a pGBI backbone. In some embodiments, a bi-shRNA sequence mentioned above can be in an expression vector, such as Doggybone (dbDNA™) DNA.

In some embodiments, the bi-shRNA sequence is comprised in a nanoplasmid, a next generation plasmid DNA with a smaller backbone, which can have lower toxicity, reduced payload activation, and fewer metabolic perturbations than canonical plasmids. Nanoplasmids are discussed in detail, for example, in Williams and Patrick, Molecular Therapy Nucleic Acids vol. 32 494-503. 7 Apr. 2023.

In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:11. In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:12. In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO: 13. In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:14. In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:15. In some embodiments, an expression vector comprising a bifunctional shRNA sequence that knocks down a patient specific EWSR1-ATF1 fusion gene (e.g., a fusion gene having at least 90% (e.g., 90%, 91%, 92%, 93%, 94%, 95%, 96%, 97%, 98%, 99%, or 100%) identity to the sequence of SEQ ID NO:3) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:16.

In some embodiments, an expression vector comprising a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO: 17. In some embodiments, an expression vector comprising a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:18. In some embodiments, an expression vector comprising a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:19. In some embodiments, an expression vector comprising a bifunctional shRNA sequence that knocks down three different EWSR1-ATF1 fusion genes (e.g., a Type 1 EWSR1-ATF1 fusion gene, a Type 2 EWSR1-ATF1 fusion gene, and a patient specific EWSR1-ATF1 fusion gene) in a pGBI backbone can comprise a sequence having at least 90% (e.g., 90%, 91%, 92%, 93%, 94%, 95%, 96%, 97%, 98%, 99%, or 100%) identity to the sequence of SEQ ID NO:20.

### Single Knockdown of EWSR1-CREB1 Fusion Gene

In some embodiments, a bi-shRNA sequence can knockdown the expression of a EWSR1-CREB1 fusion gene that comprises EWSR1 5′ end sequence up to exon 7 fused with CREB1 from exon 7 to 3′ end sequence of CREB1.

For the embodiments described above, in some embodiments, a bifunctional shRNA sequence that knocks down an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:45.

### Double Knockdown of EWSR1-ATF1 and EWSR1-CREB1 Fusion Genes

In some embodiments, a bi-shRNA sequence can knockdown the expressions of both an EWSR1-ATF1 fusion gene (e.g., a Type 1, Type 2, Type 3, Type 4, or rare fusion EWSR1-ATF1 fusion gene), and an EWSR1-CREB1 fusion gene.

In some embodiments, a bi-shRNA sequence can knockdown the expressions of a Type 1 EWSR1-ATF1 fusion gene and an EWSR1-CREB1 fusion gene. In some embodiments, a bifunctional shRNA sequence that knocks down a Type 1 EWSR1-ATF1 fusion gene and an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:46.

In some embodiments, a bi-shRNA sequence can knockdown the expressions of a Type 2 EWSR1-ATF1 fusion gene and an EWSR1-CREB1 fusion gene. In some embodiments, a bifunctional shRNA sequence that knocks down a Type 2 EWSR1-ATF1 fusion gene and an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:47.

In some embodiments, a bi-shRNA sequence can knockdown the expressions of a Type 3 EWSR1-ATF1 fusion gene and an EWSR1-CREB1 fusion gene. In some embodiments, a bifunctional shRNA sequence that knocks down a Type 3 EWSR1-ATF1 fusion gene and an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:48.

In some embodiments, a bi-shRNA sequence can knockdown the expressions of a Type 4 EWSR1-ATF1 fusion gene and an EWSR1-CREB1 fusion gene. In some embodiments, a bifunctional shRNA sequence that knocks down a Type 4 EWSR1-ATF1 fusion gene and an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:49.

In some embodiments, a bi-shRNA sequence can knockdown the expressions of a patient specific EWSR1-ATF1 fusion gene and an EWSR1-CREB1 fusion gene. A patient specific EWSR1-ATF1 fusion gene can be determined by sequencing the tumor cells in a biopsy sample from the patient. A bi-shRNA construct can then be designed that can knockdown the expressions of the patient specific EWSR1-ATF1 fusion gene and the EWSR1-CREB1 fusion gene. In some embodiments, a bifunctional shRNA sequence that knocks down a patient specific EWSR1-ATF1 fusion gene and an EWSR1-CREB1 fusion gene can comprise a sequence having at least 90% (e.g., 90%, 91%, 92%, 93%, 94%, 95%, 96%, 97%, 98%, 99%, or 100%) identity to the sequence of SEQ ID NO:50.

In some embodiments, the expression vector further comprises a promoter, e.g., a tumor specific promoter. In some embodiments, the expression vector further comprises an enhancer. In some embodiments, the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the bi-shRNA comprises or consists of two stem-loop structures each with a miR-30a loop. In some embodiments, a first stem-loop structure of the two stem-loop structures comprises complementary guiding strand and passenger strand. In some embodiments, the second stem-loop structure of the two stem-loop structures comprises three mismatches in the passenger strand. In some embodiments, the three mismatches are at positions 9 to 11 in the passenger strand. In some embodiments, the bi-shRNA comprises or consists of two stem-loop structures each with a miR-17/92 cluster backbone. In some embodiments, a first stem-loop structure of the two stem-loop structures comprises complementary guiding strand and passenger strand. In some embodiments, the second stem-loop structure of the two stem-loop structures comprises three mismatches in the passenger strand. In some embodiments, the three mismatches are at positions 9 to 11 in the passenger strand.

In some embodiments, the first nucleic acid and the second nucleic acid are operably linked to a promoter. In some embodiments, the promoter is a cytomegalovirus (CMV) promoter. In some embodiments, the CMV promoter is a mammalian CMV promoter. In some embodiments, the mammalian CMV promoter comprises a CMV immediate early (IE) 5′ UTR enhancer sequence and a CMV IE Intron A.

In some embodiments, a nucleotide sequence encoding a picornaviral 2A ribosomal skip peptide sequence is intercalated between the first and the second nucleic acid inserts.

### EXAMPLES

### Example 1—Methods

### Cell Culture

Cell culture of HEK 293 (purchased from ATCC CRL-1573, Gaithersburg, MD) were maintained in DMEM (ATCC) medium supplemented with 10% heat inactivated FBS and 2 mM L-Glutamine. KAS cells, a clear cell sarcoma cell line (provided by Dr. Nakamura of Tokyo Medical University, Tokyo, Japan) were maintained in DMEM (ATCC) medium supplemented with 10% heat inactivated and 2 mM L-Glutamine. Cell culture of clear cell sarcoma SU-CCS-1 (provided by Dr. Duckett of Moffitt Cancer Center, Tampa, FL) was maintained in RPMI (ATCC) medium supplemented with 10% heat inactivated and 2 mM L-Glutamine.

### Transfection

Cells were harvested using trypsin-EDTA. After collection, cells were resuspended in OptiMEM at a concentration of 2×10⁶/mL. 0.8×10⁶ cells were transferred to a 4 mm gap electroporation cuvette (Bio-Rad) and mixed with various concentrations of plasmid DNA for electroporation. Cells were electroporated at 200V, 1000 μF with exponential decay protocol (Bio-Rad Gene Pulser). After electroporation, cells were transferred to 6-wells plates at a concentration of 0.4×10⁶ per well with 2.0 mL of each dedicated media supplemented with 10% heat inactivated FBS and 2 mM L-Glutamine.

### Harvest, Cell Lysis, and Protein Quantification

Cells were harvested using cell scrapers and wells were rinsed with PBS. Cells were then centrifugated at 460 rcf for 10 min at room temperature. After centrifugation, supernatants were discarded, and cell pellets were then ready for lysis. MPER reagent was used for protein extraction. Pierce Detergent Compatible Bradford Kit was used for protein quantification. Using the colorimetric Bradford protein assay, standards, unknown samples, and blanks samples were run in duplicate to a 96-well plate to measure protein quantity. 96-well plate was run in the plate reader (Agilent BioTek, Santa Clara, CA) at the wavelength 595 nm for analysis.

### Western Assay

Once protein quantification was valued, samples were then prepared for electrophoresis. 20 μg of protein sample were mixed with equal volume of 4× Laemmli buffer to a new tube and boiled at 95° C. for 10 min. Tubes were then centrifugated to 16,000 g for 1 min. TGX Stain-Free Precast Gel were used to run the samples, gel was run at 75V 3A-300W for 1 h 30 m in the Bio-Rad PowerPac HC (Bio-Rad Laboratories, Inc. Ann Arbor, MI). Gel was then transferred into membrane using Trans-Blot Turbo Midi Nitrocellulose Transfer Pack and Trans-Blot Turbo Transfer System (Bio-Rad Laboratories, Inc. Ann Arbor, MI) at 1.3A, 25V for 10 minutes. The membrane was rinsed with TBS 1× buffer with 0.1% Tween for 5 min. Primary antibodies used during the first incubation were: EWRS-1: rabbit anti-EWSR1 or anti-EWSR1/EWS Rabbit monoclonal antibody, and ATF 1 Polyclonal Antibody (Cat #PIPA541490, Invitrogen, Carlsbad, CA).

### Cell Survival Assay

To measure the cell survival after EWSR1-ATF1 fusion knockdown, Cell Proliferation Kit II (XTT) from Roche was used following the manufacture protocol. Briefly, after transfection fusion knockdown plasmid, cells were plated in on clear 96-well plate at density of 4000-5000 cells per well in 50 μL growth medium. Then the desired amount of KRAS inhibitor in 50 μL growth medium was added. After incubation in 37° C., 5% CO2 incubator for 2-7 days, the XTT labeling reagent and the electron coupling reagent were thawed and mixed at ratio of 5 mL to 0.1 mL right before the experiment. Then, 50 μL XTT labeling mixture per well was added to the 96-well plate and incubate at 37° C., 5% CO₂ for 2 or 4 hours. The absorbance at 450 nm was read using a BioTek Epoch plate reader. The reference wavelength was set to 660 nm. Each treatment condition was loaded on 96-well plate at least in quadruplicate.

### RNA and DNA Isolation

Total cell RNA was isolated using Monarch® Total RNA Miniprep Kit from NEB (Ipswich, MA). First, cells were harvested from cell culture flasks using trypsin (Corning, 25-053-CI), centrifuged at 430 rcf×10 minutes. The supernatant was removed, and cells were frozen at −80° C. Total cell RNA was extracted following instructions provided by the kit. RNA was quantified using Nanodrop (Thermo Scientific). 1 g RNA was then mixed 1:1 with RNA loading dye (NEB, B0363A), incubated at 65° C. for 5 min, and electrophoresed on a 1% gel to evaluate RNA integrity.

Cellular DNA was isolated using Monarch Genomic DNA Extraction and Purification kit from NEB (Ipswich, MA). Harvested cells were extracted for DNA following instructions provided by the kit. DNA was quantified using Nanodrop (Thermo Scientific) and use for PCR analysis.

### Reverse Transcription and Polymerase Chain Reaction (PCR)

1-3 μg of total RNA was reverse transcribed with a random primer mix and the LunaScript Multiplex One-Step RT-PCR Kit (NEB; Ipswich, MA) in a ThermoFisher SimpiAmp thermocycler. 2-4 μL of cDNA was amplified by polymerase chain reaction (PCR) using primer sets designed to detect specific mRNA. PCR products were electrophoresed on a 4% agarose gel.

### Example 2—Patient Specific Rare Fusion

A patient specific rare EWSR1-ATF1 fusion was identified from patient's clear cell sarcoma tissue at Moffitt Cancer Center (Tempa, FL). A 2000 supporting sequencing reads placed an in-frame fusion between the 3′ end of exon 11 (AA 376) of EWSR1 fused with the 5′ end of exon 6 (AA 171) of ATF1. This results in a chimeric protein in which the EWSR1 promoter through exon 11 are intact and read into exon 6 of ATF1. This EWSR1-ATF1 fusion is likely oncogenic (PMID: 28481359). This chimeric protein potentially contains the EWSR1 transactivating domain linked to an ATF1 bZIP domain that is no longer regulated by cAMP and may well alter the transcriptional regulation of genes normally controlled by ATF1 in clear cell sarcomas (also called malignant melanoma of soft parts) (PMID: 8401579).

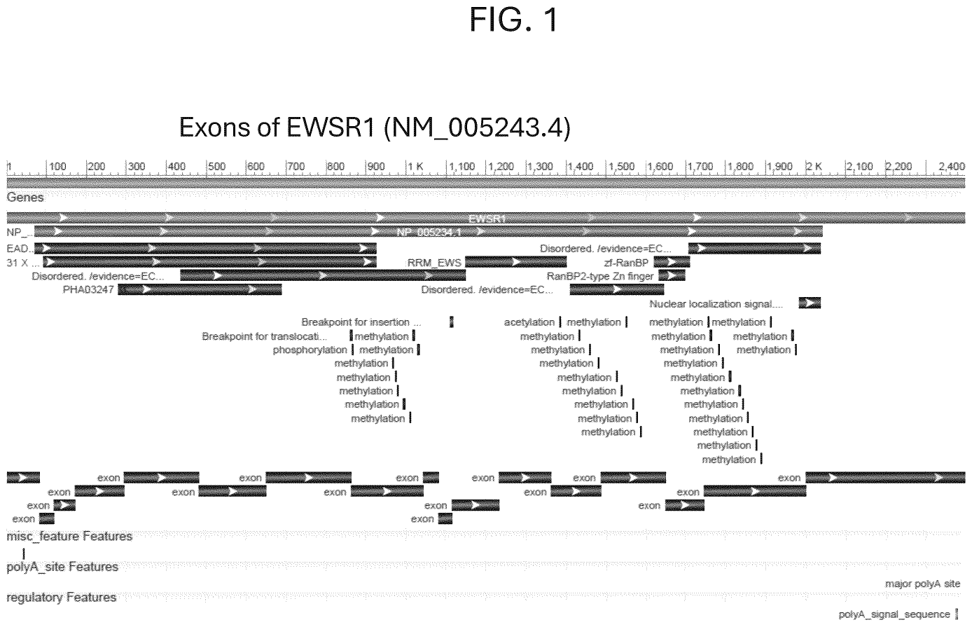

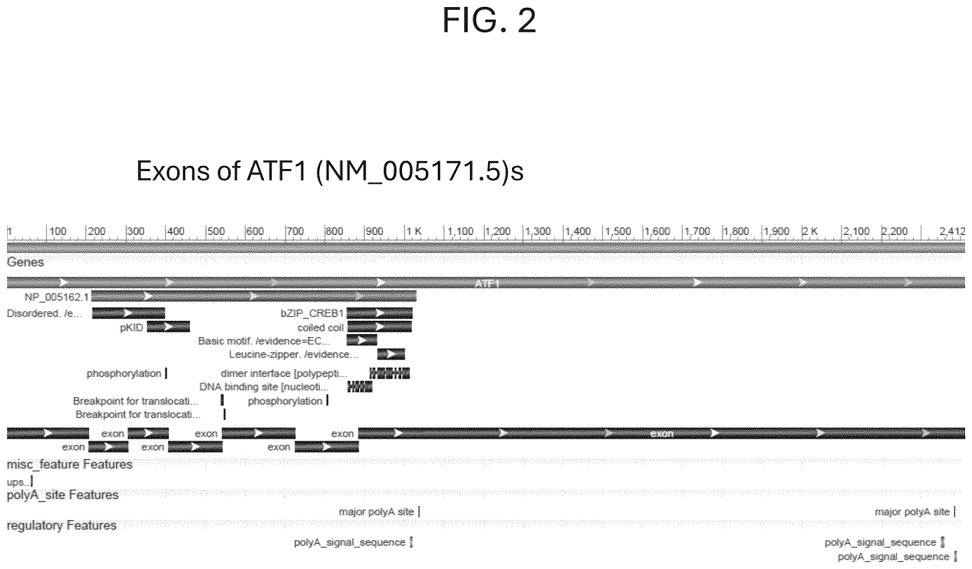

EWSR1 mRNA sequence map (RefSeq: NM_005243.4) is shown in FIG. 1 and ATF1 mRNA sequence map (RefSeq: NM 005171.5) is shown in FIG. 2. EWSR1 and ATF1 mRNA sequences (SEQ ID NOS:1 and 2, respectively) were used to compose the rare fusion mRNA joining together with designated exon joining. The predicted rare fusion mRNA sequence is shown in SEQ ID NO:3 with a theoretic 532 amino acid protein of 57 Kdal molecular weight is shown in SEQ ID NO:4.

### Example 3—Design of Rare Fusion Expression Plasmid and Bifunctional shRNA Constructs

Expression plasmid expressing the rare fusion protein was designed and constructed in pUMVC3 backbone (pGBI-2001, SEQ ID NO:9). Additional expression plasmid expressing Strep tag at the amino terminus was also designed and constructed (pGBI-2002, SEQ ID NO:10).

The bifunctional shRNA (bi-shRNA) design was targeted at the joining sequence of the rare fusion gene transcript between EWSR1 exon 11 and ATF1 exon 6. The target sequence is shown as bold in SEQ ID NO:3. Six bi-shRNA constructs were designed and constructed (pGBI-2003, pGBI-2004, pGBI-2005, pGBI-2006, pGBI-2007, and pGBI-2008) in pUMVC3 based backbone as described previously. The list of bi-shRNA for the rare fusion gene knockdowns with their respective target sequence is shown in Table 1 below. The plasmid sequences for the 6 constructs are shown in SEQ ID NOS:11-16, respectively.

TABLE 1
EWSR1/exon 11-ATF1/exon 6 rare fusion

| | | | Modifi- | |
|---|---|---|---|---|
| Plasmid | Backbone | Expression Unit | cations | Target Sequence |
| pGBI- | pUMVC3 | EWSR1-ATF1 rare fusion | None | |
| 2001 | | protein | | |
| pGBI- | pUMVC3 | EWSR1-ATF1 rare fusion | Strep Tag | |
| 2002 | | protein | | |
| pGBI- | pUMVC3 | EWSR1-ATF1 rare fusion | | CGTGGAATGGTTTGATGCT |
| 2003 | | bi-shRNA | | GCATC (SEQ ID NO: 51) |
| pGBI- | pUMVC3 | EWSR1-ATF1 rare fusion | | CCGTGGAATGGTTTGATGC |
| 2004 | | bi-shRNA | | TGCAT (SEQ ID NO: 52) |
| pGBI- | pUMVC3 | EWSR1-ATF1 rare fusion | | GCCGTGGAATGGTTTGATG |
| 2005 | | bi-shRNA | | CTGCA (SEQ ID NO: 53) |
| pGBI- | pUMVC3 | EWSR1-ATF1 rare fusion | | TGCCGTGGAATGGTTTGAT |
| 2006 | | bi-shRNA | | GCTGC (SEQ ID NO: 54) |
| pGBI- | pUMVC3 | EWSR1-ATF1 rare fusion | | CTGCCGTGGAATGGTTTGA |
| 2007 | | bi-shRNA | | TGCTG (SEQ ID NO: 55) |
| pGBI- | pUMVC3 | EWSR1-ATF1 rare fusion | Replace | TGCCGTGGAATGGTTTGAT |
| 2008 | | bi-shRNA | Intron A | GCTGC (SEQ ID NO: 56) |

### Example 4—Test Rare Fusion Specific Knockdown Analyzed by RT-PCR

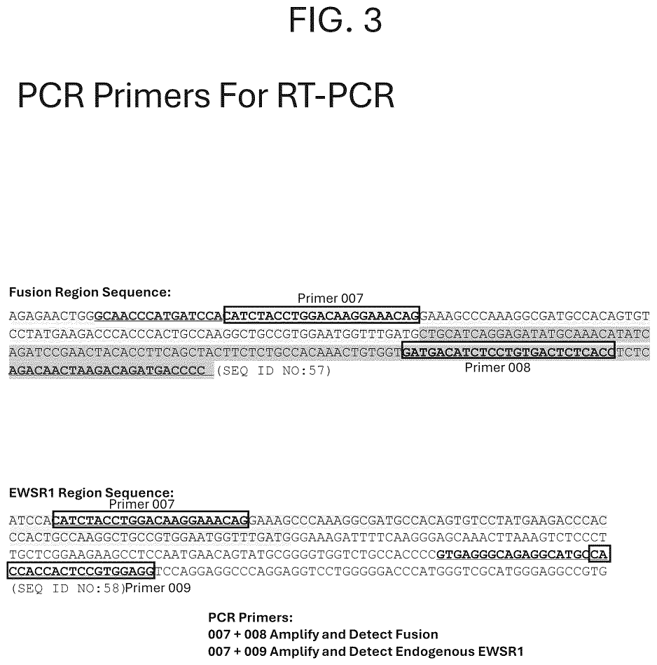

To demonstrate activity and specificity of designed bi-shRNA knockdown, HEK-293 cells were co-transfected with rare fusion expression plasmid (pGBI-2001) and each of the bi-shRNA constructs. HEK-293 expresses endogenous wild-type EWSR1 without translocation fusion which served as test control for the specificity of fusion specific bi-shRNA constructs knockdown. At 24 or 48 hours post transfection, total RNA was isolated from the transfected cells. The mRNA for the rare fusion protein and for the endogenous wild-type EWSR1 can be identified via RT-PCR with either fusion specific primer set (EWSR-ATF007 and EWSR-ATF008) or EWSR1 wild-type specific primer set (EWSR-ATF007 and EWSR-ATF009). The sequences and locations of primer sets are shown in FIG. 3.

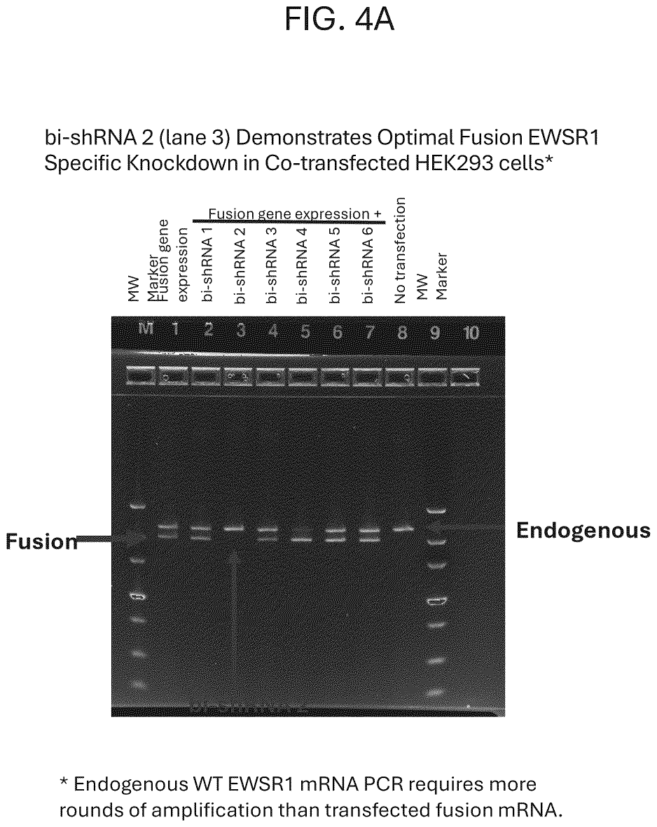

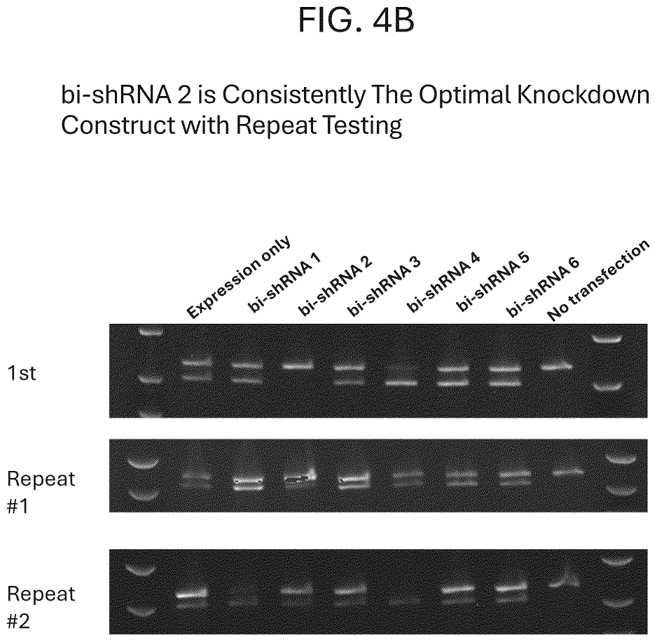

Transgene fusion mRNA was present in much higher abundance than the endogenous wild-type EWSR1 mRNA; with the same cDNA, the transgene fusion mRNA was readily detected with 16 cycles of PCR amplification, while endogenous wild-type mRNA required 28 cycles of amplification. Because the difference in PCR product size, the PCR products from rare fusion amplification and wild-type EWSR1 amplification were loaded onto one gel for expression comparison. An example of RT-PCR result is shown in FIGS. 4A and 4B. Lane 1 of FIG. 4B shows RNA from rare fusion expression construct (pGBI-2001) transfection only. Lane 8 shows HEK-293 cell RNA without transfection. Lane 1 has an additional rare fusion PCR product below the endogenous EWSR1 wild-type PCR product (lane 8). Lanes 2-7 show co-transfection with each of the designed bi-shRNA constructs (pGBI-2003, pGBI-2004, pGBI-2005, pGBI-2006, pGBI-2007, and pGBI-2008) with rare fusion expression construct (pGBI-2001). As shown, pGBI-2003 (lane 2, bi-shRNA 1), pGBI-2007 (lane 6, bi-shRNA 5), and pGBI-2008 (lane 7, bi-shRNA 6) co-transfection did not affect the expression of either rare fusion or endogenous wild-type EWSR1, while pGBI-2004 (lane 3, bi-shRNA 2) showed disappearance of rare fusion specific PCR product with the retention of wild-type EWSR1. On the other hand, pGBI-2006 (lane 5, bi-shRNA 4) showed the disappearance of wild-type EWSR1 without affecting the rare fusion expression.

In summary, comparatively, pGBI-2004 (bi-shRNA 2) appeared to be the best construct for rare fusion specific knockdown without affecting the endogenous wild-type EWSR1. The results were repeated two more times with similar outcome as shown on FIG. 4B. Noted that the analysis was performed with optimized end-point PCR. The analysis method was meant to show the comparative advantage of each bi-shRNA construct in rare fusion specific knockdown. The analysis may not quantitatively demonstrate the efficiency of knockdown.

### Example 5—Triple Knockdown Constructs

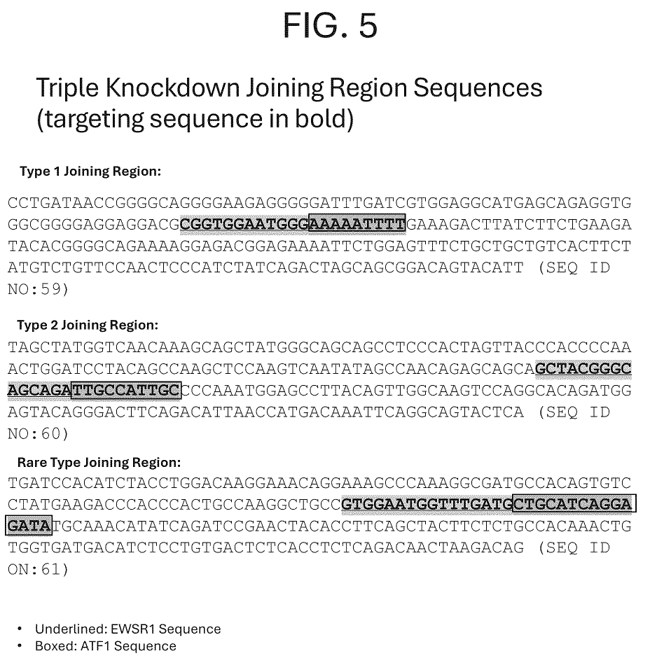

Four triple knockdown bi-shRNA constructs were designed and made to enable knockdown of Type 1, Type 2, and the rare EWSR1-ATF1 fusion with single plasmid. The targeting sequences for Type 1, Type 2, and the rare fusion are shown in FIG. 5. The Type 1 and Type 2 sequences are put together according to Type 1 and Type 2 translocations identified by Wang et al. The EWSR1 mRNA sequence (RefSeq: NM_005243.4) and ATF1 mRNA sequence (RefSeq: NM 005171.5) as shown in SEQ ID NO:1 and SEQ ID NO:2 were used to compose the rare fusion mRNA joining together with designated exon joining. The predicted mRNA sequences for Type 1 and Type 2 fusion are shown in SEQ ID NO:5 and SEQ ID NO:7, respectively, with a theoretic predicted 531 amino acids and 426 amino acids, respectively. Predicted amino acid sequences are shown in SEQ ID NO:4 and SEQ ID NO:6, respectively.

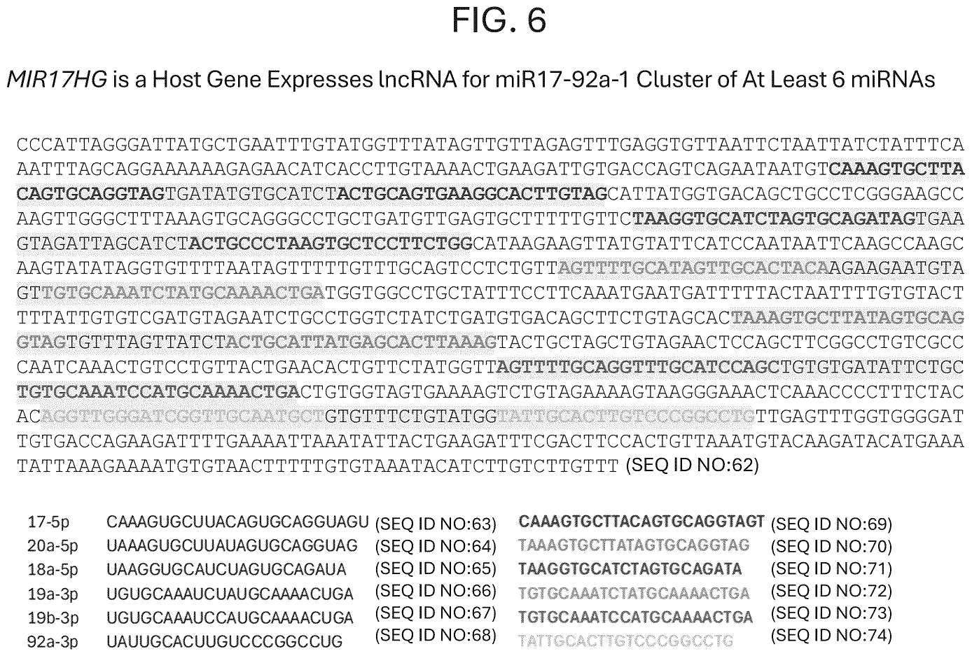

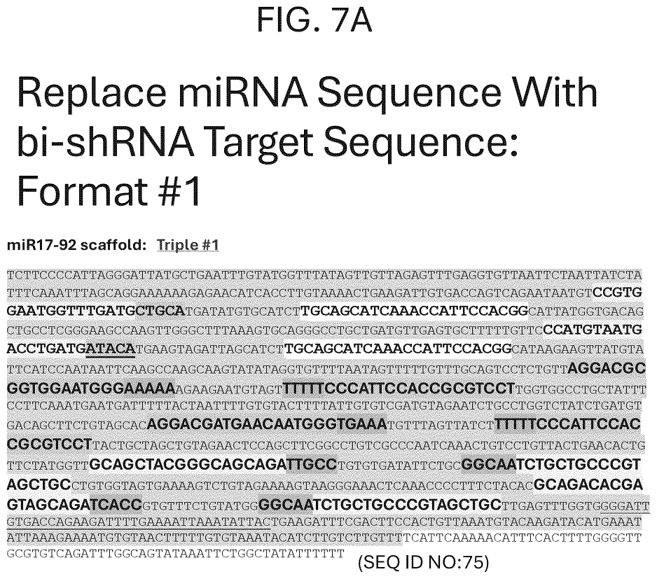

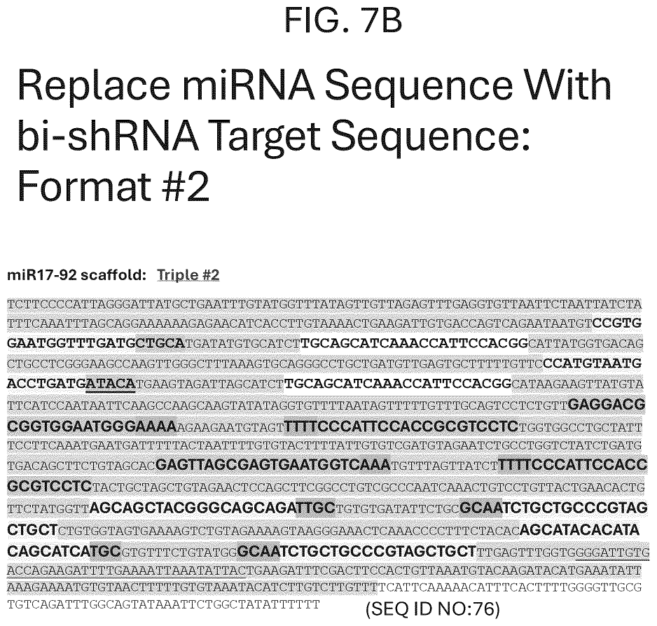

The triple knockdown was designed in the scaffold of miR17-92a cluster of 6 miRNAs (FIG. 6). Four triple knockdown bi-shRNA constructs were designed and constructed. The target sequences specific for Type 1 and Type 2 EWSR1-ATF1 fusion junction sequences are shown in FIG. 5. Bi-shRNA from pGBI-2004 was adopted for rare fusion knockdown component of the triple constructs for all four triple knockdown constructs. The four triple knockdown constructs contained varied combinations of Type 1 and Type 2 knockdown components. Triple bi-shRNA constructs were designed and constructed; two bi-shRNA sequences formats are shown in FIGS. 7A and 7B. The four triple knockdown constructs were named pGBI-2011, pGBI-2012, pGBI-2013, and pGBI-2014; their respective sequences are shown in SEQ ID NOS:17-20.

As discussed in the knockdown analysis in Example 4 above, a single nucleotide shift could result in significant differences in RNAi activity. Thus, for the EWSR1-ATF1 triple knockdown constructs, the knockdown activity of two EWSR1-ATF1 Type 1 targeting sequences and two EWSR1-ATF1 Type 2 targeting sequences were compared.

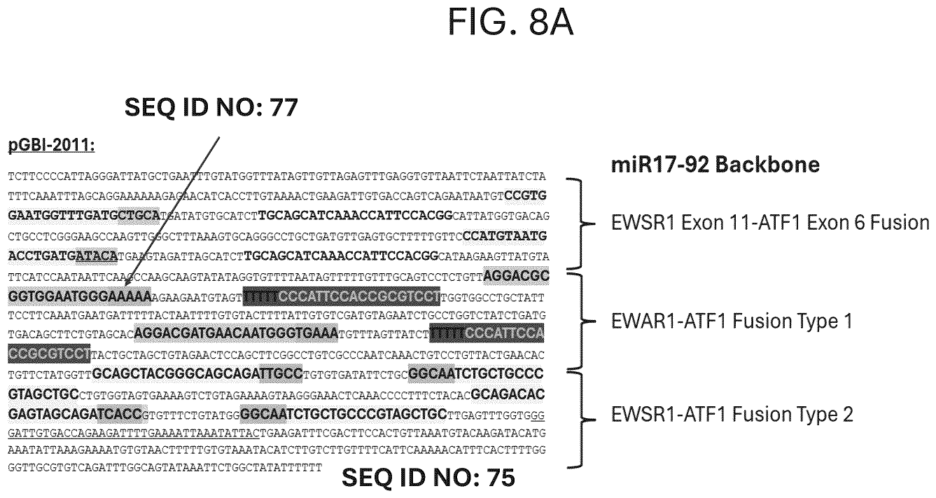

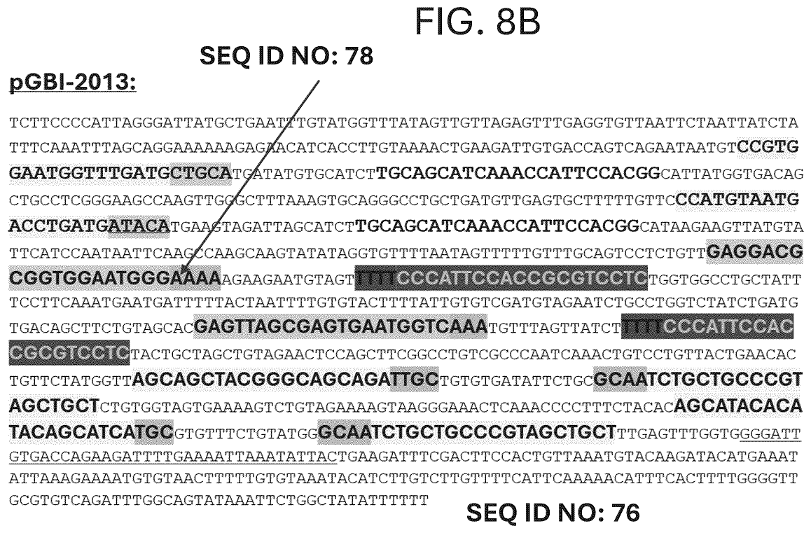

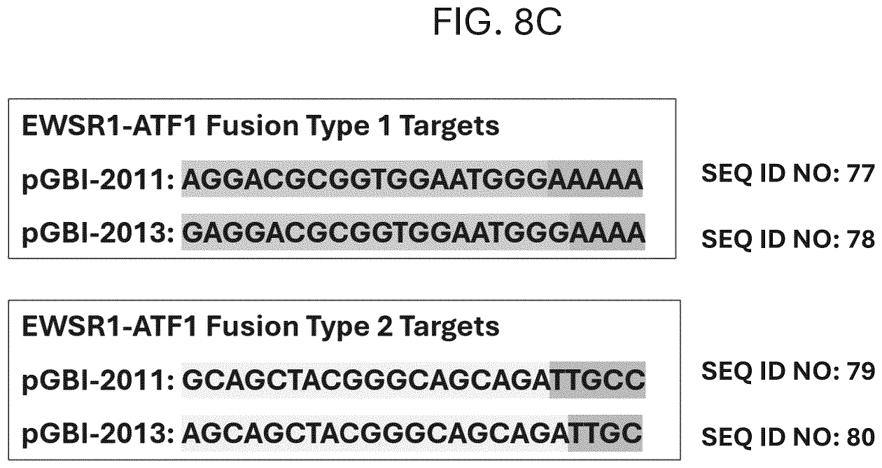

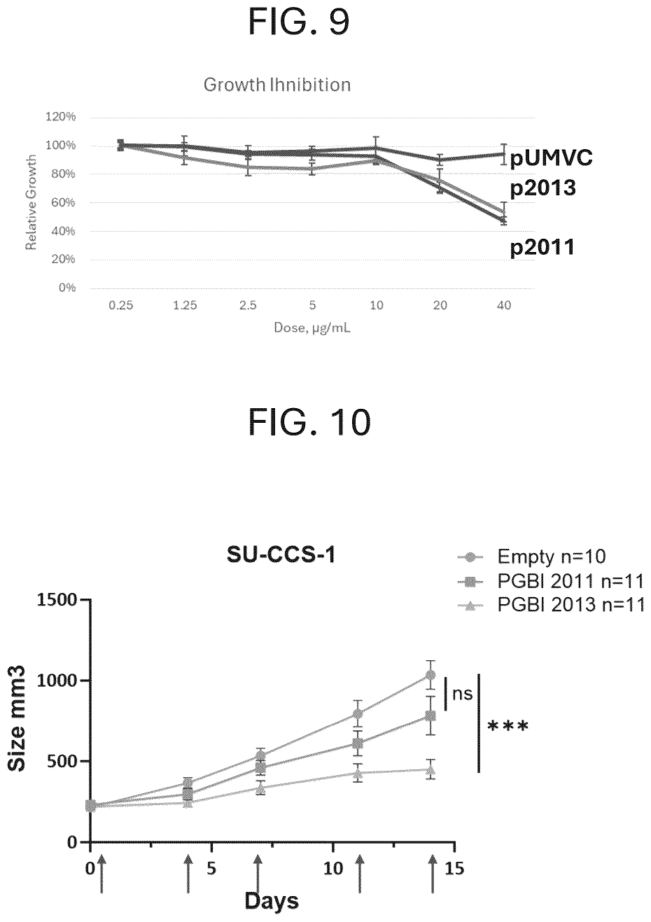

The Type 1 targeting sequence in pGBI-2011 is 5′ AGGACGCGGTGGAATGGGAAAAA 3′ (SEQ ID NO: 77). The Type 1 targeting sequence in pGBI-p2013 is 5′ GAGGACGCGGTGGAATGGGAAAA 3′ (SEQ ID NO: 78). The Type 2 targeting sequence in pGBI-2011 is 5′ GCAGCTACGGGCAGCAGATTGCC 3′ (SEQ ID NO: 79). The Type 2 targeting sequence in pGBI-p2013 is 5′ AGCAGCTACGGGCAGCAGATTGC 3′ (SEQ ID NO: 80). The targeting sequences are also shown in FIG. 8. The bolded portions of SEQ ID NO: 77-80 are from EWSR1. The italicized portions of SEQ ID NO: 70-80 are from ATF1. The joining sequence is placed at the seed region with single nucleotide shift between pGBI-2011 and pGBI-2013.

With a preliminary growth inhibition study with Type 1 fusion cell SU-CCS-1, three constructs pGBI-2011, pGBI-2012, and pGBI-2013 inhibited SU-CCS-1 cell growth in a dose responsive manner. Marked growth inhibition was observed in relationship to dose>20 μg/ml compared to empty plasmid control (pUMVC; FIG. 8). Earlier timepoints show that pGBI-2013 causes slightly greater growth inhibition than pGBI-2011. Further analysis was undertaken for pGBI-2013 and pGBI-2014.

KAS cells were initially considered as Type 2 fusion cells. However, expression characteristics of KAS cells were identified as the Type 3 cell line. Alternatively, the Hegwa-CCS-1 cell line is a Type 2 fusion cell line that may be used. See, e.g., Outani et al., BMC Cancer, 2014. Currently, cell lines for rare type fusion are not available for growth inhibition tests. The development of additional Type 2 and rare fusion type cell lines from SU-CCS-1 cell utilizing CRISPR technology are underway.

### Example 6—In Vivo Efficacy of the EWSR1-ATF1 Type 1 Fusion Knockdown Plasmid in a SU-CCS-1 Xenograft Model

### 6.1—DOTAP:Cholesterol Liposomes for Delivery of the Knockdown Plasmid

The EWSR1-ATF1 Type 1 fusion knockdown plasmid was delivered using DOTAP:cholesterol liposomes. The liposomes are complexed with payload, which, in this example, is the knockdown plasmid, to produce a DNA-LPX product. The DOTAP:cholesterol formulation used herein is described in detail in Rao, D. D., et al., Mol Ther, 2016. 24(8): p. 1412-22 [4]. Briefly, DOTAP and cholesterol powders were dissolved in ethanol and then injected into an aqueous solution (D5W) to create the raw liposomes. The raw liposomes were buffer exchanged to remove the ethanol, and the volume was adjusted to create the stock of 10 mM DOTAP, 9.0 mM Cholesterol, in D5W buffer, with <0.5% residual ethanol.

The plasmid DNA payload was diluted to 0.8 mg DNA/mL and then mixed with stock liposomes (1:1 mix ratio) to create the DNA-LPX product. The final product concentration was 5.0 mM DOTAP, 4.5 mM cholesterol, and 0.4 mg DNA/mL in D5W buffer.

QC testing of the DNA-LPX product included: safety (sterility, endotoxin, and mycoplasma), lipid identity (thin layer chromatography), DNA identity (restriction digest), DNA concentration (Abs260) purity and physical properties (e.g., OD400, particle size, polydispersity index, and zeta potential).

6.2— Knockdown Plasmids pGBI-2011 and pGBI-2013 in Lipoplex Provided a Survival Advantage in Xenograft Mouse Model

Thirty-nine 6-9 week old female NSG mice were subcutaneously injected in the flank with 10 million SU-CCS-1 cells per mouse to establish xenografted tumor. Treatment with knockdown plasmid started when the tumor reached 150 mm³ volume. Tumor bearing mice were treated twice weekly (indicated with arrows in FIG. 10) via tail vein infusion with either empty liposome (carrier control), 25 μg of pGBI-2011 per mouse, or 25 μg of pGBI-2013 per mouse. Tumor sizes were monitored twice weekly with digital calipers. Treatments ended when tumor volume was 2 cm³. As shown in FIG. 10, tumor size was reduced as a result of treatment with DNA-LPX product containing the knockdown plasmid pGBI-2011 or pGBI-2013, with pGBI 2013 as more effective than pGBI 2011.

6.3—Administration of Knockdown Plasmid pGBI-2013 Using Implanted Vascular Access Buttons™ (VABs™) Demonstrated In Vivo Tumor Growth Reduction in Xenograft Mouse Model

In the previous study described in the section 6.2 above, the results were encouraging given early evidence of survival advantage after 4 successful dose administrations. Unfortunately, on day 14 infusion (the fifth treatment; FIG. 10), the tail vein infusion became unstable and swelling of the tail veins caused leakage and inefficient delivery of the DNA-LPX product. Thus, an alternative mouse continuous infusion system with vascular access Buttons™ (VABs™) was used in subsequent studies. The VABs are implanted through the jugular vein which enables more stable vascular access and product delivery enabling prolonged repeat administration without complications.

A pilot study was set up to treat xenograft mice using VABs™ and to observe treatment efficacy with the new infusion method. Based on the study described in section 6.2 above, pGBI-2013 was the superior knockdown plasmid and was selected for this study.

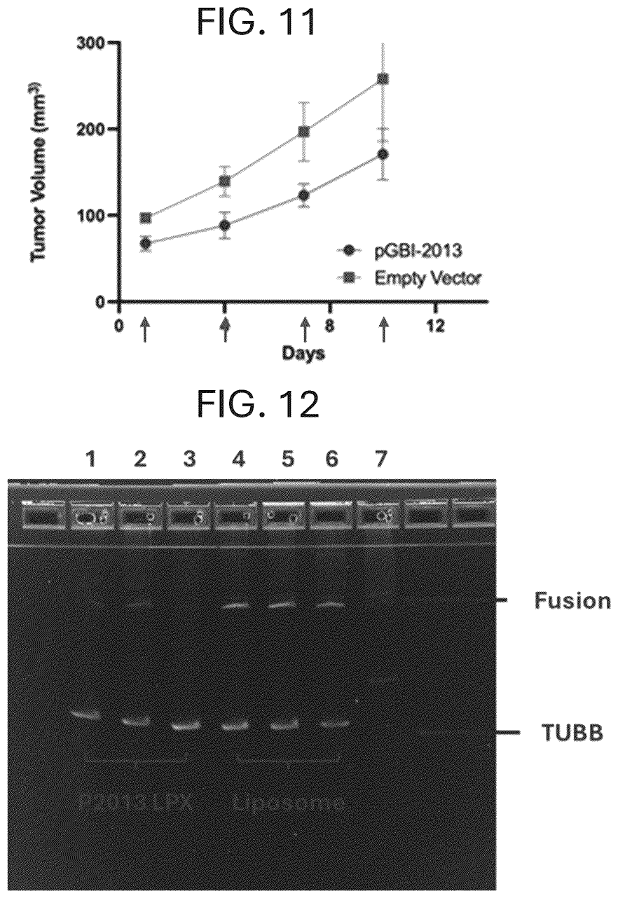

Six tumor bearing study mice per group were implanted with VABs™ for delivery of DNA-LPX product with pGBI-2013. The mice were treated twice weekly (indicated with arrows in FIG. 11) with either empty liposome (control, no plasmid) or 25 μg pGBI-2013 per mouse.

On day 12, tumors were explanted from the mice and total RNA were isolated from tumor samples for RT-PCR analysis. 0.25 μg of total RNA were used for RT-PCR with primer sets specific for β-Tubulin (TUBB) or EWSR1 Exon 11-ATF1 Exon 6 Type 1 fusion (Type 1 fusion) at 21 cycles or 24 cycles, respectively. Aliquots of RT-PCR products were run onto a 4% agarose gel. pGBI-2013 LPX treated samples have greater reduction in mRNA of the Type 1 fusion in comparison to empty liposome treatment control (FIG. 12). The reduction in Type 1 fusion mRNA (FIG. 12) correlates with tumor volume reduction (FIG. 11) in pGBI-2013 LPX treated samples.

Taken together, the in vivo results demonstrate (1) successful reduction in tumor volume and (2) mRNA expression EWSR1 Exon 11-ATF1 Exon 6 Type 1 fusion in mice treated with the pGBI-2013 knockdown plasmid targeting the Type 1 fusion.

### REFERENCES

1. Barve, M., et al., Phase 1 Trial of Bi-shRNA STMN1 BIV in Refractory Cancer.

Molecular Therapy, 2015. 23(6): p. 1123-1130.
2. Jay, C., et al., Preclinical assessment of wt GNE gene plasmid for management of hereditary inclusion body myopathy 2 (HIBM2). Gene Regul Syst Bio, 2008. 2: p. 243-52.
3. Phadke, A. P., et al., Safety and in vivo expression of a GNE-transgene: a novel treatment approach for hereditary inclusion body myopathy-2. Gene Regul Syst Bio, 2009. 3: p. 89-101.
4. Rao, D. D., et al., Preclinical Justification of pbi-shRNA EWS/FLI1 Lipoplex (LPX) Treatment for Ewing's Sarcoma. Mol Ther, 2016. 24(8): p. 1412-22.
5. Rao, D. D., et al., KRAS mutant allele-specific expression knockdown in pancreatic cancer model with systemically delivered bi-shRNA KRAS lipoplex. PLoS One, 2018. 13(5): p. e0193644.
6. Wang, Z., et al., Preclinical Biodistribution and Safety Evaluation of a pbi-shRNA STMN1 Lipoplex after Subcutaneous Delivery. Toxicological Sciences, 2016. 155(2): p. 400-408.
7. Rocconi, R. P., et al., Gemogenovatucel-T (Vigil) immunotherapy as maintenance in frontline stage III/IV ovarian cancer (VITAL): a randomised, double-blind, placebo-controlled, phase 2b trial. Lancet Oncol, 2020. 21(12): p. 1661-1672.
8. Rocconi, R. P., et al., Gemogenovatucel-T (Vigil) immunotherapy demonstrates clinical benefit in homologous recombination proficient (HRP) ovarian cancer. Gynecol Oncol, 2021. 161(3): p. 676-680.
9. Walter, A., et al., Gemogenovatucel-T (Vigil) maintenance immunotherapy: 3-year survival benefit in homologous recombination proficient (HRP) ovarian cancer. Gynecol Oncol, 2021. 163(3): p. 459-464.
10. Yu, J., et al., PDX1 associated therapy in translational medicine. Ann Transl Med, 2016. 4(11): p. 214.
11. Nemunaitis, J., et al., Gemogenovatucel-T (Vigil): bi-shRNA plasmid-based targeted immunotherapy. Future Oncol, 2024: p. 1-16.
12. Nemunaitis, J., et al., Clonal Neoantigen: Emerging “Mechanism-based” Biomarker of Immunotherapy Response. Cancers (Basel), 2023. 15(23): p. 5616.
13. Morand, S., et al., Clinical characterization and therapeutic targeting of fusion genes in oncology. Future Oncol, 2025. 21(10): p. 1249-1260.
14. Davies, K. D. and R. C. Doebele, Molecular pathways: ROS1 fusion proteins in cancer. Clin Cancer Res, 2013. 19(15): p. 4040-5.
15. Taniue, K. and N. Akimitsu, Fusion Genes and RNAs in Cancer Development. Noncoding RNA, 2021. 7(1).
16. Hanahan, D., Hallmarks of Cancer: New Dimensions. Cancer Discov, 2022. 12(1): p. 31-46.
17. Quiroga, I. Y., et al., Oncogenic fusion proteins and their role in three-dimensional chromatin structure, phase separation, and cancer. Curr Opin Genet Dev, 2022. 74: p. 101901.
18. Reyes-Sandoval, A. and H. C. Ertl, CpG methylation of a plasmid vector results in extended transgene product expression by circumventing induction of immune responses. Molecular Therapy, 2004. 9(2): p. 249-261.
19. Klinman, D. M., Immunotherapeutic uses of CpG oligodeoxynucleotides. Nature Reviews Immunology, 2004. 4(4): p. 249-259.
20. Scott, V. L., et al., Novel synthetic plasmid and Doggybone DNA vaccines induce neutralizing antibodies and provide protection from lethal influenza challenge in mice. Hum Vaccin Immunother, 2015. 11(8): p. 1972-82.
21. Walters, A. A., et al., Comparative analysis of enzymatically produced novel linear DNA constructs with plasmids for use as DNA vaccines. Gene Ther, 2014. 21(7): p. 645-52.
22. Thornton, C. D., et al., Safe and stable generation of induced pluripotent stem cells using doggybone DNA vectors. Mol Ther Methods Clin Dev, 2021. 23: p. 348-358.
23. Hocar, O., et al., Clear Cell Sarcoma (Malignant Melanoma) of Soft Parts: A Clinicopathologic Study of 52 Cases. Dermatology Research and Practice, 2012. 2012: p. 984096.
24. Ibrahim, R. M., S. Steenstrup Jensen, and J. Juel, Clear cell sarcoma-A review.

Journal of Orthopaedics, 2018. 15(4): p. 963-966.
25. Gonzaga, M. I., et al., The epidemiology and survivorship of clear cell sarcoma: a National Cancer Database (NCDB) review. Journal of Cancer Research and Clinical Oncology, 2018. 144(9): p. 1711-1716.
26. Designating an Orphan Product: Drugs and Biological Products. 2024 Aug. 12, 2024 [cited 2025; Available from: https://www.fda.gov/industry/medical-products-rare-diseases-and-conditions/designating-orphan-product-drugs-and-biological-products.
27. FDA Advances Rare Disease Drug Development with New Evidence Principles. 2025 Sep. 3, 2025 [cited 2025; Available from: https://www.fda.gov/news-events/press-announcements/fda-advances-rare-disease-drug-development-new-evidence-principles.
28. Smrke, A., et al., Systemic treatment of advanced clear cell sarcoma: results from a retrospective international series from the World Sarcoma Network. ESMO Open, 2022. 7(3): p. 100522.
29. Takita, J., The role of anaplastic lymphoma kinase in pediatric cancers. Cancer Sci, 2017. 108(10): p. 1913-1920.
30. Gounder, M. M., et al., Clinical genomic profiling in the management of patients with soft tissue and bone sarcoma. Nature Communications, 2022. 13(1): p. 3406.
31. Treatment Response and Conditional Survival in Advanced Pancreatic Cancer Patients Treated with FOLFIRINOX: A Multicenter Cohort Study—PMC.
32. Patane, S., et al., MET Overexpression Turns Human Primary Osteoblasts into Osteosarcomas. Cancer Research, 2006. 66(9): p. 4750-4757.
33. Yang, J., et al., Genetic amplification of the vascular endothelial growth factor (VEGF) pathway genes, including VEGFA, in human osteosarcoma. Cancer, 2011. 117(21): p. 4925-38.
34. Tang, H., et al., Fusion genes in cancers: Biogenesis, functions, and therapeutic implications. Genes Dis, 2025. 12(5): p. 101536.
35. Latysheva, N. S. and M. M. Babu, Molecular Signatures of Fusion Proteins in Cancer. ACS Pharmacol Transl Sci, 2019. 2(2): p. 122-133.
36. Nemunaitis, J., et al., Clonal Neoantigen: Emerging “Mechanism Based” Biomarker of Immunotherapy Response. Cancers, 2023. 15(23): p. 5616.
37. Franzetti, G. A., et al., Cell-to-cell heterogeneity of EWSR1-FLI1 activity determines proliferation/migration choices in Ewing sarcoma cells. Oncogene, 2017. 36(25): p. 3505-3514.
38. Bailey, K. M., et al., EWS-FLI1 low Ewing sarcoma cells demonstrate decreased susceptibility to T-cell-mediated tumor cell apoptosis. Oncotarget, 2019. 10(36): p. 3385-3399.
39. McGranahan, N., et al., Clonal neoantigens elicit T cell immunoreactivity and sensitivity to immune checkpoint blockade. Science, 2016. 351(6280): p. 1463-9.
40. Litchfield, K., et al., Meta-analysis of tumor- and T cell-intrinsic mechanisms of sensitization to checkpoint inhibition. Cell, 2021. 184(3): p. 596-614 e14.
41. Boll, L. M., et al., The impact of mutational clonality in predicting the response to immune checkpoint inhibitors in advanced urothelial cancer. Sci Rep, 2023. 13(1): p. 15287.
42. Rizvi, N. A., et al., Cancer immunology. Mutational landscape determines sensitivity to PD-1 blockade in non-small cell lung cancer. Science, 2015. 348(6230): p. 124-8.
43. Riaz, N., et al., The role of neoantigens in response to immune checkpoint blockade.

Int Immunol, 2016. 28(8): p. 411-9.
44. Simmons, O., et al., Ewing's Sarcoma: Development of RNA Interference-Based Therapy for Advanced Disease. ISRN Oncol, 2012. 2012: p. 247657.
45. Ghisoli, M., et al., Innovative Exploratory Clinical Approaches for Relapsed and/or Refractory Metastatic Ewing's Sarcoma. Clin Oncol., 2016. 1: p. 1079.
46. Rao, D. D., et al., siRNA vs. shRNA: similarities and differences. Adv Drug Deliv Rev, 2009. 61(9): p. 746-59.
47. Liu, S. H., et al., PDX-1: demonstration of oncogenic properties in pancreatic cancer. Cancer, 2011. 117(4): p. 723-33.
48. Jo, V., EWSR1 fusions: Ewing sarcoma and beyond. Cancer Cytopathology, 2020. 128.
49. Dermawan, J. K., et al., Comprehensive genomic profiling of EWSR1/FUS::CREB translocation-associated tumors uncovers prognostically significant recurrent genetic alterations and methylation-transcriptional correlates. Mod Pathol, 2022. 35(8): p. 1055-1065.
50. Panagopoulos, I., et al., Molecular genetic characterization of the EWS/ATF1 fusion gene in clear cell sarcoma of tendons and aponeuroses. International Journal of Cancer, 2002. 99(4): p. 560-567.
51. Wang, W.-L., et al., Detection and characterization of EWSR1/ATF1 and EWSR1/CREB1 chimeric transcripts in clear cell sarcoma (melanoma of soft parts). Modern Pathology, 2009. 22(9): p. 1201-1209.
52. Antonescu, C. R., et al., EWS-CREB1: A Recurrent Variant Fusion in Clear Cell Sarcoma-Association with Gastrointestinal Location and Absence of Melanocytic Differentiation. Clinical Cancer Research, 2006. 12(18): p. 5356-5362.
53. Cunningham, D. S., et al., Factors affecting plasmid production in Escherichia coli from a resource allocation standpoint. Microb Cell Fact, 2009. 8: p. 27.
54. Mucker, E. M., et al., SARS-CoV-2 Doggybone DNA Vaccine Produces Cross-Variant Neutralizing Antibodies and Is Protective in a COVID-19 Animal Model. Vaccines (Basel), 2022. 10(7).
55. Jay, C. M., et al., Assessment of dbDNATM GNEwt/bi-shRNA-GNEM743T Lipoplex for GNE Myopathy: Improved Potency and Safety. Journal of Gene Medicine, (Submitted).
56. Kerneis, F., et al., Preclinical Assessment of GNEwt/bi-shRNA-GNEM743T Lipoplex Product Development for GNE Myopathy. Hum Gene Ther, (Submitted).
57. Mae, H., et al., Targeting the Clear Cell Sarcoma Oncogenic Driver Fusion Gene EWSR1::ATF1 by HDAC Inhibition. Cancer Res Commun, 2023. 3(7): p. 1152-1165.
58. Kataria, B., et al., Pazopanib in rare histologies of metastatic soft tissue sarcoma. Ecancermedicalscience, 2021. 15: p. 1281.
59. Urakawa, H., et al., Phase II trial of pazopanib in patients with metastatic or unresectable chemoresistant sarcomas: A Japanese Musculoskeletal Oncology Group study. Cancer Sci, 2020. 111(9): p. 3303-3312.
60. Cho, H. J., et al., Durvalumab plus pazopanib combination in patients with advanced soft tissue sarcomas: a phase II trial. Nature Communications, 2024. 15(1): p. 685.
61. Gounder, M., et al., Tazemetostat in advanced epithelioid sarcoma with loss of INI1/SMARCB1: an international, open-label, phase 2 basket study. Lancet Oncol, 2020. 21(11): p. 1423-1432.
62. Schoffski, P., et al., Activity and safety of crizotinib in patients with advanced clear-cell sarcoma with MET alterations: European Organization for Research and Treatment of Cancer phase II trial 90101 ‘CREATE’. Ann Oncol, 2017. 28(12): p. 3000-3008.
63. Stacchiotti, S., et al., Tumor response to sunitinib malate observed in clear-cell sarcoma. Ann Oncol, 2010. 21(5): p. 1130-1.
64. Sidlik Muskatel, R., et al., Case report: Robust response of metastatic clear cell sarcoma treated with cabozantinib and immunotherapy. Front Pediatr, 2022. 10: p. 940927.
65. Nakai, T., et al., Trabectedin is a promising antitumor agent potentially inducing melanocytic differentiation for clear cell sarcoma. Cancer Med, 2017. 6(9): p. 2121-2130.
66. Herzog, J., et al., Trabectedin Followed by Irinotecan Can Stabilize Disease in Advanced Translocation-Positive Sarcomas with Acceptable Toxicity. Sarcoma, 2016. 2016: p. 7461783.
67. Outani, Hidetatsu et al. “Establishment of a novel clear cell sarcoma cell line (Hewga-CCS), and investigation of the antitumor effects of pazopanib on Hewga-CCS.” BMC Cancer vol. 14 455. 19 Jun. 2014, doi:10.1186/1471-2407-14-455.

INFORMAL SEQUENCE LISTING

| SEQ ID | | |
|---|---|---|
| NO | SEQUENCE | Notes |
| 1 | GCAGTGCGGCGCCTAGAGGGAAAGCGAGAGGGAGACGGACGTTGAGAGAA | EWSR1 mRNA |
| | CGAGGAGGAAGGAGAGAAAATGGCGTCCACGGATTACAGTACCTATAGCCAA | sequence |
| | GCTGCAGCGCAGCAGGGCTACAGTGCTTACACCGCCCAGCCCACTCAAGGAT | (NM_005243.4 |
| | ATGCACAGACCACCCAGGCATATGGGCAACAAAGCTATGGAACCTATGGACA | Homo sapiens EWS |
| | GCCCACTGATGTCAGCTATACCCAGGCTCAGACCACTGCAACCTATGGGCAGA | RNA binding |
| | CCGCCTATGCAACTTCTTATGGACAGCCTCCCACTGGTTATACTACTCCAACTG | protein 1 (EWSR1), |
| | CCCCCCAGGCATACAGCCAGCCTGTCCAGGGGTATGGCACTGGTGCTTATGAT | transcript variant 2, |
| | ACCACCACTGCTACAGTCACCACCACCCAGGCCTCCTATGCAGCTCAGTCTGC | mRNA) |
| | ATATGGCACTCAGCCTGCTTATCCAGCCTATGGGCAGCAGCCAGCAGCCACTG | |
| | CACCTACAAGACCGCAGGATGGAAACAAGCCCACTGAGACTAGTCAACCTCA | |
| | ATCTAGCACAGGGGGTTACAACCAGCCCAGCCTAGGATATGGACAGAGTAAC | |
| | TACAGTTATCCCCAGGTACCTGGGAGCTACCCCATGCAGCCAGTCACTGCACC | |
| | TCCATCCTACCCTCCTACCAGCTATTCCTCTACACAGCCGACTAGTTATGATCA | |
| | GAGCAGTTACTCTCAGCAGAACACCTATGGGCAACCGAGCAGCTATGGACAG | |
| | CAGAGTAGCTATGGTCAACAAAGCAGCTATGGGCAGCAGCCTCCCACTAGTT | |
| | ACCCACCCCAAACTGGATCCTACAGCCAAGCTCCAAGTCAATATAGCCAACA | |
| | GAGCAGCAGCTACGGGCAGCAGAGTTCATTCCGACAGGACCACCCCAGTAGC | |
| | ATGGGTGTTTATGGGCAGGAGTCTGGAGGATTTTCCGGACCAGGAGAGAACC | |
| | GGAGCATGAGTGGCCCTGATAACCGGGGCAGGGGAAGAGGGGGATTTGATCG | |
| | TGGAGGCATGAGCAGAGGTGGGCGGGGAGGAGGACGCGGTGGAATGGGCA | |
| | GCGCTGGAGAGCGAGGTGGCTTCAATAAGCCTGGTGGACCCATGGATGAAGG | |
| | ACCAGATCTTGATCTAGGCCCACCTGTAGATCCAGATGAAGACTCTGACAACA | |
| | GTGCAATTTATGTACAAGGATTAAATGACAGTGTGACTCTAGATGATCTGGCA | |
| | GACTTCTTTAAGCAGTGTGGGGTTGTTAAGATGAACAAGAGAACTGGGCAAC | |
| | CCATGATCCACATCTACCTGGACAAGGAAACAGGAAAGCCCAAAGGCGATGC | |
| | CACAGTGTCCTATGAAGACCCACCCACTGCCAAGGCTGCCGTGGAATGGTTT | |
| | GATGGGAAAGATTTTCAAGGGAGCAAACTTAAAGTCTCCCTTGCTCGGAAGA | |
| | AGCCTCCAATGAACAGTATGCGGGGTGGTCTGCCACCCCGTGAGGGCAGAGG | |
| | CATGCCACCACCACTCCGTGGAGGTCCAGGAGGCCCAGGAGGTCCTGGGGG | |
| | ACCCATGGGTCGCATGGGAGGCCGTGGAGGAGATAGAGGAGGCTTCCCTCCA | |
| | AGAGGACCCCGGGGTTCCCGAGGGAACCCCTCTGGAGGAGGAAACGTCCAG | |
| | CACCGAGCTGGAGACTGGCAGTGTCCCAATCCGGGTTGTGGAAACCAGAACT | |
| | TCGCCTGGAGAACAGAGTGCAACCAGTGTAAGGCCCCAAAGCCTGAAGGCT | |
| | TCCTCCCGCCACCCTTTCCGCCCCCGGGTGGTGATCGTGGCAGAGGTGGCCC | |
| | TGGTGGCATGCGGGGAGGAAGAGGTGGCCTCATGGATCGTGGTGGTCCCGGT | |
| | GGAATGTTCAGAGGTGGCCGTGGTGGAGACAGAGGTGGCTTCCGTGGTGGC | |
| | CGGGGCATGGACCGAGGTGGCTTTGGTGGAGGAAGACGAGGTGGCCCTGGG | |
| | GGGCCCCCTGGACCTTTGATGGAACAGATGGGAGGAAGAAGAGGAGGACGT | |
| | GGAGGACCTGGAAAAATGGATAAAGGCGAGCACCGTCAGGAGCGCAGAGAT | |
| | CGGCCCTACTAGATGCAGAGACCCCGCAGAGCTGCATTGACTACCAGATTTAT | |
| | TTTTTAAACCAGAAAATGTTTTAAATTTATAATTCCATATTTATAATGTTGGCCA | |
| | CAACATTATGATTATTCCTTGTCTGTACTTTAGTATTTTTCACCATTTGTGAAGA | |
| | AACATTAAAACAAGTTAAATGGTAGTGTGCGGAGTTTTTTTTTCTTCCTTCTTT | |
| | TAAAAATGGTTGTTTAAGACTTTAACAATGGGAACCCCTTGTGAGCATGCTCA | |
| | GTATCATTGTGGAGAACCAAGAGGGCCTCTTAACTGTAACAATGTTCATGGTT | |
| | GTGATGTTTTTTTTTTTTTTTTAAATAAAATTCCAAATGTTTATAAAGA | |
| 2 | AGTAGCGGCCCTGACTGCCGAGGAAACGGTAGCTTAGGACAGTTGGCTGTTA | ATF1 mRNA |
| | AGTGACACTGATTCTCCCCGCCCCGCCTGACCCCCGAGAAGGAGGCTTGTCC | sequence |
| | CCCGCTGCGTGAGGGGGTGGGGAAGTGGGTAGTGAATTCGGATCTACCTGGG | (NM_005171.5:1- |
| | AGGGGGGAGTGGAAGTTCCCGCCCCGGAGAGCGGCGAGGCGGCAGCCACA | 2412 Homo sapiens |
| | GTTGATTATGGAAGATTCCCACAAGAGTACCACGTCAGAGACAGCACCTCAA | activating |
| | CCTGGTTCAGCAGTTCAGGGAGCTCACATTTCTCATATTGCTCAACAGGTATC | transcription factor |
| | ATCTTTATCAGAAAGTGAGGAGTCCCAGGACTCATCCGACAGCATAGGCTCCT | 1 (ATF1), transcript |
| | CACAGAAAGCCCACGGGATCCTAGCACGGCGCCCATCTTACAGAAAAATTTT | variant 3, mRNA) |
| | GAAAGACTTATCTTCTGAAGATACACGGGGCAGAAAAGGAGACGGAGAAAA | |
| | TTCTGGAGTTTCTGCTGCTGTCACTTCTATGTCTGTTCCAACTCCCATCTATCA | |
| | GACTAGCAGCGGACAGTACATTGCCATTGCCCCAAATGGAGCCTTACAGTTGG | |
| | CAAGTCCAGGCACAGATGGAGTACAGGGACTTCAGACATTAACCATGACAAA | |
| | TTCAGGCAGTACTCAGCAAGGTACAACTATTCTTCAGTATGCACAGACCTCTG | |
| | ATGGACAGCAGATACTTGTGCCCAGCAATCAGGTGGTCGTACAAACTGCATC | |
| | AGGAGATATGCAAACATATCAGATCCGAACTACACCTTCAGCTACTTCTCTGC | |
| | CACAAACTGTGGTGATGACATCTCCTGTGACTCTCACCTCTCAGACAACTAAG | |
| | ACAGATGACCCCCAATTGAAAAGAGAAATAAGGTTAATGAAAAACAGAGAA | |
| | GCTGCTCGAGAATGTCGCAGAAAGAAGAAAGAATATGTGAAATGCCTGGAAA | |
| | ACCGAGTTGCAGTCCTGGAAAATCAAAATAAAACTCTAATAGAAGAGTTAAA | |
| | AACTTTGAAGGATCTTTATTCCAATAAAAGTGTTTGATTCCTAAGAAAGAAAA | |
| | TATTTTTGTGGACATGCATAAAAATTAAATGGATTTCCTAGTGGAGTTTTATAA | |
| | ATTAAAAGGTCAAAACTGAAGCTTTTTATTTAGGCTTTTCCAAATCAAGGATA | |
| | AATATCTTACGCACGATATCTAGTGACAGAGGAGAAAGTGGAAAATGACCTCA | |
| | AGGAAGCTACGGGCACAACTGGAAGCTTTGTAGAAATTAAACATATTCAAGG | |
| | AGCAAGAAATGAACTTTCAGCAGTCTAAATTTTCTAAATAACCAATAGTTGCC | |
| | AATCTAAAATGGCAGAGAAGATGAAATTTGATAAACTGAATTTTTTTTAAAAA | |
| | TCCATTTACCCTACAGGTTTGCATTTGTTTGCTGAAATTTACCTTTTTTTAGTTA | |
| | TATATATGTGTGTGTGTGTGTGTAATTTCTGCCAATAAATTCTAAATTACAAAGG | |
| | TAAGAGAAAACCTAGTACATTACTAAATATATAAAGTATATGTTCTGATTATGTA | |
| | TACTTGTTCTAGTGTCAAGTCTTTTTAAGTGGGTTTTTAAAAGTTTGTTATTGG | |
| | ACTTGAATGGATTTTTGAGACTAGGTTAATTATTTTTGAGGTCTTATCCTAAAA | |
| | GGCATCTAAGGTACATGAATGGAGTATGGTGATTTTATAACATTTTTTATCAGA | |
| | ATGGAAAAAGAACTGTTTAAAAGTTTGATACTTTTAAATAGTTGGTTTTTTTGC | |
| | TTACTCTGGTAATGATTTTCTACAAATACATAATAAATTGTTTTTTTGAGTCTAT | |
| | ATTCTGTATGCAGTTGAATATCCATTACTTATTCTGCTGTGCTTTAATAGAATGG | |
| | AATGTTTACAGGCCCTTAAAATATTATTTTTAAAAAACCTTCTGAAGATACATA | |
| | CCAAAGTTTTTCCAAGAAGATTTTATAATCAATTTAATAATGTAAGGTTTATCA | |
| | GATTCTATAATAGTATAGTTATTAAGGCAATTTTATGTTAGAGACTATTTTGTAAT | |
| | GTAGTGAGTGGTACCTTTATAAGAAAAGTGACTGCCAATATATTTTTATAGCTG | |
| | ATCTTTATAAATTCTAATGTTGAGTTTTTAATGATTATTTTAAATGTTTATATAGT | |
| | TTAGTAAAATTTGCATCTCAAAGTATCATTTTTATATTATGGGACGTTTTCAGAT | |
| | TGGCTAATATTTGCATTGTAAATTTTGTATGCAGTTTATCTAAAATTCAAAAATA | |
| | CTGTCAGTACACCAGCGTTTAACATCTATATTCCAATTTGTATACAGTTTAAAAT | |
| | TGTACTGCAAAACTATTGTGTGCTCTTACACAGTATGCATCATATTGTTGTCTG | |
| | TGAAATTAAAGGACATTTGATAGTCTACTGAATGTAAAATATAATGCTTGGTAT | |
| | ATGAATGATAA | |
| 3 | GCAGTGCGGCGCCTAGAGGGAAAGCGAGAGGGAGACGGACGTTGAGAGAA | EWSR1-ATF1 rare |
| | CGAGGAGGAAGGAGAGAAAATGGCGTCCACGGATTACAGTACCTATAGCCAA | fusion mRNA |
| | GCTGCAGCGCAGCAGGGCTACAGTGCTTACACCGCCCAGCCCACTCAAGGAT | sequence (targeting |
| | ATGCACAGACCACCCAGGCATATGGGCAACAAAGCTATGGAACCTATGGACA | region in bold) |
| | GCCCACTGATGTCAGCTATACCCAGGCTCAGACCACTGCAACCTATGGGCAGA | |
| | CCGCCTATGCAACTTCTTATGGACAGCCTCCCACTGGTTATACTACTCCAACTG | |
| | CCCCCCAGGCATACAGCCAGCCTGTCCAGGGGTATGGCACTGGTGCTTATGAT | |
| | ACCACCACTGCTACAGTCACCACCACCCAGGCCTCCTATGCAGCTCAGTCTGC | |
| | ATATGGCACTCAGCCTGCTTATCCAGCCTATGGGCAGCAGCCAGCAGCCACTG | |
| | CACCTACAAGACCGCAGGATGGAAACAAGCCCACTGAGACTAGTCAACCTCA | |
| | ATCTAGCACAGGGGGTTACAACCAGCCCAGCCTAGGATATGGACAGAGTAAC | |
| | TACAGTTATCCCCAGGTACCTGGGAGCTACCCCATGCAGCCAGTCACTGCACC | |
| | TCCATCCTACCCTCCTACCAGCTATTCCTCTACACAGCCGACTAGTTATGATCA | |
| | GAGCAGTTACTCTCAGCAGAACACCTATGGGCAACCGAGCAGCTATGGACAG | |
| | CAGAGTAGCTATGGTCAACAAAGCAGCTATGGGCAGCAGCCTCCCACTAGTT | |
| | ACCCACCCCAAACTGGATCCTACAGCCAAGCTCCAAGTCAATATAGCCAACA | |
| | GAGCAGCAGCTACGGGCAGCAGAGTTCATTCCGACAGGACCACCCCAGTAGC | |
| | ATGGGTGTTTATGGGCAGGAGTCTGGAGGATTTTCCGGACCAGGAGAGAACC | |
| | GGAGCATGAGTGGCCCTGATAACCGGGGCAGGGGAAGAGGGGGATTTGATCG | |
| | TGGAGGCATGAGCAGAGGTGGGCGGGGAGGAGGACGCGGTGGAATGGGCA | |
| | GCGCTGGAGAGCGAGGTGGCTTCAATAAGCCTGGTGGACCCATGGATGAAGG | |
| | ACCAGATCTTGATCTAGGCCCACCTGTAGATCCAGATGAAGACTCTGACAACA | |
| | GTGCAATTTATGTACAAGGATTAAATGACAGTGTGACTCTAGATGATCTGGCA | |
| | GACTTCTTTAAGCAGTGTGGGGTTGTTAAGATGAACAAGAGAACTGGGCAAC | |
| | CCATGATCCACATCTACCTGGACAAGGAAACAGGAAAGCCCAAAGGCGATGC | |
| | CACAGTGTCCTATGAAGACCCACCCACTGCCAAGGCTGCCGTGGAATGGTTT | |
| | GATGCTGCATCAGGAGATATGCAAACATATCAGATCCGAACTACACCTTCAG | |
| | CTACTTCTCTGCCACAAACTGTGGTGATGACATCTCCTGTGACTCTCACCTCT | |
| | CAGACAACTAAGACAGATGACCCCCAATTGAAAAGAGAAATAAGGTTAATGA | |
| | AAAACAGAGAAGCTGCTCGAGAATGTCGCAGAAAGAAGAAAGAATATGTGA | |
| | AATGCCTGGAAAACCGAGTTGCAGTCCTGGAAAATCAAAATAAAACTCTAAT | |
| | AGAAGAGTTAAAAACTTTGAAGGATCTTTATTCCAATAAAAGTGTTTGATTCC | |
| | TAAGAAAGAAAATATTTTTGTGGACATGCATAAAAATTAAATGGATTTCCTAGT | |
| | GGAGTTTTATAAATTAAAAGGTCAAAACTGAAGCTTTTTATTTAGGCTTTTCCA | |
| | AATCAAGGATAAATATCTTACGCACGATATCTAGTGACAGAGGAGAAAGTGGA | |
| | AAATGACCTCAAGGAAGCTACGGGCACAACTGGAAGCTTTGTAGAAATTAAA | |
| | CATATTCAAGGAGCAAGAAATGAACTTTCAGCAGTCTAAATTTTCTAAATAAC | |
| | CAATAGTTGCCAATCTAAAATGGCAGAGAAGATGAAATTTGATAAACTGAATT | |
| | TTTTTTAAAAATCCATTTACCCTACAGGTTTGCATTTGTTTGCTGAAATTTACCT | |
| | TTTTTTAGTTATATATATGTGTGTGTGTGTGTGTAATTTCTGCCAATAAATTCTAA | |
| | ATTACAAAGGTAAGAGAAAACCTAGTACATTACTAAATATATAAAGTATATGTT | |
| | CTGATTATGTATACTTGTTCTAGTGTCAAGTCTTTTTAAGTGGGTTTTTAAAAG | |
| | TTTGTTATTGGACTTGAATGGATTTTTGAGACTAGGTTAATTATTTTTGAGGTCT | |
| | TATCCTAAAAGGCATCTAAGGTACATGAATGGAGTATGGTGATTTTATAACATT | |
| | TTTTATCAGAATGGAAAAAGAACTGTTTAAAAGTTTGATACTTTTAAATAGTTG | |
| | GTTTTTTTGCTTACTCTGGTAATGATTTTCTACAAATACATAATAAATTGTTTTT | |
| | TTGAGTCTATATTCTGTATGCAGTTGAATATCCATTACTTATTCTGCTGTGCTTT | |
| | AATAGAATGGAATGTTTACAGGCCCTTAAAATATTATTTTTAAAAAACCTTCTG | |
| | AAGATACATACCAAAGTTTTTCCAAGAAGATTTTATAATCAATTTAATAATGTA | |
| | AGGTTTATCAGATTCTATAATAGTATAGTTATTAAGGCAATTTTATGTTAGAGAC | |
| | TATTTTGTAATGTAGTGAGTGGTACCTTTATAAGAAAAGTGACTGCCAATATAT | |
| | TTTTATAGCTGATCTTTATAAATTCTAATGTTGAGTTTTTAATGATTATTTTAAAT | |
| | GTTTATATAGTTTAGTAAAATTTGCATCTCAAAGTATCATTTTTATATTATGGGAC | |
| | GTTTTCAGATTGGCTAATATTTGCATTGTAAATTTTGTATGCAGTTTATCTAAAA | |
| | TTCAAAAATACTGTCAGTACACCAGCGTTTAACATCTATATTCCAATTTGTATA | |
| | CAGTTTAAAATTGTACTGCAAAACTATTGTGTGCTCTTACACAGTATGCATCAT | |
| | ATTGTTGTCTGTGAAATTAAAGGACATTTGATAGTCTACTGAATGTAAAATATA | |
| | ATGCTTGGTATATGAATGATAA | |
| 4 | MASTDYSTYSQAAAQQGYSAYTAQPTQGYAQTTQAYGQQSYGTYGQPTDVSY | EWSR1-ATF1 rare |
| | TQAQTTATYGQTAYATSYGQPPTGYTTPTAPQAYSQPVQGYGTGAYDTTTATVTT | fusion amino acid |
| | TQASYAAQSAYGTQPAYPAYGQQPAATAPTRPQDGNKPTETSQPQSSTGGYNQPS | sequence (532 aa) |
| | LGYGQSNYSYPQVPGSYPMQPVTAPPSYPPTSYSSTQPTSYDQSSYSQQNTYGQP | |
| | SSYGQQSSYGQQSSYGQQPPTSYPPQTGSYSQAPSQYSQQSSSYGQQSSFRQDHP | |
| | SSMGVYGQESGGFSGPGENRSMSGPDNRGRGRGGFDRGGMSRGGRGGGRGG | |
| | MGSAGERGGFNKPGGPMDEGPDLDLGPPVDPDEDSDNSAIYVQGLNDSVTLDD | |
| | LADFFKQCGVVKMNKRTGQPMIHIYLDKETGKPKGDATVSYEDPPTAKAAVEW | |
| | FDAASGDMQTYQIRTTPSATSLPQTVVMTSPVTLTSQTTKTDDPQLKREIRLMKN | |
| | REAARECRRKKKEYVKCLENRVAVLENQNKTLIEELKTLKDLYSNKSV | |
| 5 | GCAGTGCGGCGCCTAGAGGGAAAGCGAGAGGGAGACGGACGTTGAGAGAA | Predicted Type 1 |
| | CGAGGAGGAAGGAGAGAAAATGGCGTCCACGGATTACAGTACCTATAGCCAA | EWSR1-ATF1 |
| | GCTGCAGCGCAGCAGGGCTACAGTGCTTACACCGCCCAGCCCACTCAAGGAT | fusion mRNA |
| | ATGCACAGACCACCCAGGCATATGGGCAACAAAGCTATGGAACCTATGGACA | sequence |
| | GCCCACTGATGTCAGCTATACCCAGGCTCAGACCACTGCAACCTATGGGCAGA | |
| | CCGCCTATGCAACTTCTTATGGACAGCCTCCCACTGGTTATACTACTCCAACTG | |
| | CCCCCCAGGCATACAGCCAGCCTGTCCAGGGGTATGGCACTGGTGCTTATGAT | |
| | ACCACCACTGCTACAGTCACCACCACCCAGGCCTCCTATGCAGCTCAGTCTGC | |
| | ATATGGCACTCAGCCTGCTTATCCAGCCTATGGGCAGCAGCCAGCAGCCACTG | |
| | CACCTACAAGACCGCAGGATGGAAACAAGCCCACTGAGACTAGTCAACCTCA | |
| | ATCTAGCACAGGGGGTTACAACCAGCCCAGCCTAGGATATGGACAGAGTAAC | |
| | TACAGTTATCCCCAGGTACCTGGGAGCTACCCCATGCAGCCAGTCACTGCACC | |
| | TCCATCCTACCCTCCTACCAGCTATTCCTCTACACAGCCGACTAGTTATGATCA | |
| | GAGCAGTTACTCTCAGCAGAACACCTATGGGCAACCGAGCAGCTATGGACAG | |
| | CAGAGTAGCTATGGTCAACAAAGCAGCTATGGGCAGCAGCCTCCCACTAGTT | |
| | ACCCACCCCAAACTGGATCCTACAGCCAAGCTCCAAGTCAATATAGCCAACA | |
| | GAGCAGCAGCTACGGGCAGCAGAGTTCATTCCGACAGGACCACCCCAGTAGC | |
| | ATGGGTGTTTATGGGCAGGAGTCTGGAGGATTTTCCGGACCAGGAGAGAACC | |
| | GGAGCATGAGTGGCCCTGATAACCGGGGCAGGGGAAGAGGGGGATTTGATCG | |
| | TGGAGGCATGAGCAGAGGTGGGCGGGGAGGAGGACGCGGTGGAATGGGAA | |
| | AAATTTTGAAAGACTTATCTTCTGAAGATACACGGGGCAGAAAAGGAGACGG | |
| | AGAAAATTCTGGAGTTTCTGCTGCTGTCACTTCTATGTCTGTTCCAACTCCCAT | |
| | CTATCAGACTAGCAGCGGACAGTACATTGCCATTGCCCCAAATGGAGCCTTAC | |
| | AGTTGGCAAGTCCAGGCACAGATGGAGTACAGGGACTTCAGACATTAACCAT | |
| | GACAAATTCAGGCAGTACTCAGCAAGGTACAACTATTCTTCAGTATGCACAGA | |
| | CCTCTGATGGACAGCAGATACTTGTGCCCAGCAATCAGGTGGTCGTACAAACT | |
| | GCATCAGGAGATATGCAAACATATCAGATCCGAACTACACCTTCAGCTACTTC | |
| | TCTGCCACAAACTGTGGTGATGACATCTCCTGTGACTCTCACCTCTCAGACAA | |
| | CTAAGACAGATGACCCCCAATTGAAAAGAGAAATAAGGTTAATGAAAAACAG | |
| | AGAAGCTGCTCGAGAATGTCGCAGAAAGAAGAAAGAATATGTGAAATGCCTG | |
| | GAAAACCGAGTTGCAGTCCTGGAAAATCAAAATAAAACTCTAATAGAAGAGT | |
| | TAAAAACTTTGAAGGATCTTTATTCCAATAAAAGTGTTTGATTCCTAAGAAAG | |
| | AAAATATTTTTGTGGACATGCATAAAAATTAAATGGATTTCCTAGTGGAGTTTT | |
| | ATAAATTAAAAGGTCAAAACGAAGCTTTTTATTTAGGCTTTTCCAAATCAAGG | |
| | ATAAATATCTTACGCACGATATCTAGTGACAGAGGAGAAAGTGGAAAATGACC | |
| | TCAAGGAAGCTACGGGCACAACTGGAAGCTTTGTAGAAATTAAACATATTCA | |
| | AGGAGCAAAAATGAACTTTCAGCAGTCTAAATTTTCTAAATAACCAATAGTTG | |
| | CCAATCTAAAATGGCAGAGAAGATGAAATTTGATAAACTGAATTTTTTTTAAA | |
| | AATCCATTTACCCTACAGGTTTGCATTTGTTTGCTGAAATTTACCTTTTTTTAGT | |
| | TATATATATGTGTGTGTGTGTGTGTAATTTCTGCCAATAAATTCTAAATTACAAA | |
| | GGTAAGAGAAAACCTAGTACATTACTAAATATATAAAGTATATGTTCTGATTATG | |
| | TATACTTGTTCTAGTGTCAAGTCTTTTTAAGTGGGTTTTTAAAAGTTTGTTATTG | |
| | GACTTGAATGGATTTTTGAGACTAGGTTAATTATTTTTGAGGTCTTATCCTAAA | |
| | AGGCATCTAAGGTACATGAATGGAGTATGGTGATTTTATAACATTTTTTATCAG | |
| | AATGGAAAAAGAACTGTTTAAAAGTTTGATACTTTTAAATAGTTGGTTTTTTTG | |
| | CTTACTCTGGTAATGATTTTCTACAAATACATAATAAATTGTTTTTTTGAGTCTA | |
| | TATTCTGTATGCAGTTGAATATCCATTACTTATTCTGCTGTGCTTTAATAGAATG | |
| | GAATGTTTACAGGCCCTTAAAATATTATTTTTAAAAAACCTTCTGAAGATACAT | |
| | ACCAAAGTTTTTCCAAGAAGATTTTATAATCAATTTAATAATGTAAGGTTTATC | |
| | AGATTCTATAATAGTATAGTTATTAAGGCAATTTTATGTTAGAGACTATTTTGTA | |
| | ATGTAGTGAGTGGTACCTTTATAAGAAAAGTGACTGCCAATATATTTTTATAGC | |
| | TGATCTTTATAAATTCTAATGTTGAGTTTTTAATGATTATTTTAAATGTTTATATA | |
| | GTTTAGTAAAATTTGCATCTCAAAGTATCATTTTTATATTATGGGACGTTTTCAG | |
| | ATTGGCTAATATTTGCATTGTAAATTTTGTATGCAGTTTATCTAAAATTCAAAAA | |
| | TACTGTCAGTACACCAGCGTTTAACATCTATATTCCAATTTGTATACAGTTTAAA | |
| | ATTGTACTGCAAAACTATTGTGTGCTCTTACACAGTATGCATCATATTGTTGTCT | |
| | GTGAAATTAAAGGACATTTGATAGTCTACTGAATGTAAAATATAATGCTTGGTA | |
| | TATGAATGATAA | |
| 6 | MASTDYSTYSQAAAQQGYSAYTAQPTQGYAQTTQAYGQQSYGTYGQPTDVSY | Predicted Type 1 |
| | TQAQTTATYGQTAYATSYGQPPTGYTTPTAPQAYSQPVQGYGTGAYDTTTATVTT | EWSR1-ATF1 |
| | TQASYAAQSAYGTQPAYPAYGQQPAATAPTRPQDGNKPTETSQPQSSTGGYNQPS | fusion amino acid |
| | LGYGQSNYSYPQVPGSYPMQPVTAPPSYPPTSYSSTQPTSYDQSSYSQQNTYGQP | sequence (531 aa) |
| | SSYGQQSSYGQQSSYGQQPPTSYPPQTGSYSQAPSQYSQQSSSYGQQSSFRQDHP | |
| | SSMGVYGQESGGFSGPGENRSMSGPDNRGRGRGGFDRGGMSRGGRGGGRGG | |
| | MGKILKDLSSEDTRGRKGDGENSGVSAAVTSMSVPTPIYQTSSGQYIAIAPNGAL | |
| | QLASPGTDGVQGLQTLTMTNSGSTQQGTTILQYAQTSDGQQILVPSNQVVVQTA | |
| | SGDMQTYQIRTTPSATSLPQTVVMTSPVTLTSQTTKTDDPQLKREIRLMKNREAA | |
| | RECRRKKKEYVKCLENRVAVLENQNKTLIEELKTLKDLYSNKSV | |
| 7 | GCAGTGCGGCGCCTAGAGGGAAAGCGAGAGGGAGACGGACGTTGAGAGAA | Predicted Type 2 |
| | CGAGGAGGAAGGAGAGAAAATGGCGTCCACGGATTACAGTACCTATAGCCAA | EWSR1-ATF1 |
| | GCTGCAGCGCAGCAGGGCTACAGTGCTTACACCGCCCAGCCCACTCAAGGAT | fusion mRNA |
| | ATGCACAGACCACCCAGGCATATGGGCAACAAAGCTATGGAACCTATGGACA | sequence |
| | GCCCACTGATGTCAGCTATACCCAGGCTCAGACCACTGCAACCTATGGGCAGA | |
| | CCGCCTATGCAACTTCTTATGGACAGCCTCCCACTGGTTATACTACTCCAACTG | |
| | CCCCCCAGGCATACAGCCAGCCTGTCCAGGGGTATGGCACTGGTGCTTATGAT | |
| | ACCACCACTGCTACAGTCACCACCACCCAGGCCTCCTATGCAGCTCAGTCTGC | |
| | ATATGGCACTCAGCCTGCTTATCCAGCCTATGGGCAGCAGCCAGCAGCCACTG | |
| | CACCTACAAGACCGCAGGATGGAAACAAGCCCACTGAGACTAGTCAACCTCA | |
| | ATCTAGCACAGGGGGTTACAACCAGCCCAGCCTAGGATATGGACAGAGTAAC | |
| | TACAGTTATCCCCAGGTACCTGGGAGCTACCCCATGCAGCCAGTCACTGCACC | |
| | TCCATCCTACCCTCCTACCAGCTATTCCTCTACACAGCCGACTAGTTATGATCA | |
| | GAGCAGTTACTCTCAGCAGAACACCTATGGGCAACCGAGCAGCTATGGACAG | |
| | CAGAGTAGCTATGGTCAACAAAGCAGCTATGGGCAGCAGCCTCCCACTAGTT | |
| | ACCCACCCCAAACTGGATCCTACAGCCAAGCTCCAAGTCAATATAGCCAACA | |
| | GAGCAGCAGCTACGGGCAGCAGATTGCCATTGCCCCAAATGGAGCCTTACAG | |
| | TTGGCAAGTCCAGGCACAGATGGAGTACAGGGACTTCAGACATTAACCATGA | |
| | CAAATTCAGGCAGTACTCAGCAAGGTACAACTATTCTTCAGTATGCACAGACC | |
| | TCTGATGGACAGCAGATACTTGTGCCCAGCAATCAGGTGGTCGTACAAACTG | |
| | CATCAGGAGATATGCAAACATATCAGATCCGAACTACACCTTCAGCTACTTCTC | |
| | TGCCACAAACTGTGGTGATGACATCTCCTGTGACTCTCACCTCTCAGACAACT | |
| | AAGACAGATGACCCCCAATTGAAAAGAGAAATAAGGTTAATGAAAAACAGA | |
| | GAAGCTGCTCGAGAATGTCGCAGAAAGAAGAAAGAATATGTGAAATGCCTGG | |
| | AAAACCGAGTTGCAGTCCTGGAAAATCAAAATAAAACTCTAATAGAAGAGTT | |
| | AAAAACTTTGAAGGATCTTTATTCCAATAAAAGTGTTTGATTCCTAAGAAAGA | |
| | AAATATTTTTGTGGACATGCATAAAAATTAAATGGATTTCCTAGTGGAGTTTTA | |
| | TAAATTAAAAGGTCAAAACTGAAGCTTTTTATTTAGGCTTTTCCAAATCAAGG | |
| | ATAAATATCTTACGCACGATATCTAGTGACAGAGGAGAAAGTGGAAAATGACC | |
| | TCAAGGAAGCTACGGGCACAACTGGAAGCTTTGTAGAAATTAAACATATTCA | |
| | AGGAGCAAGAAATGAACTTTCAGCAGTCTAAATTTTCTAAATAACCAATAGTT | |
| | GCCAATCTAAAATGGCAGAGAAGATGAAATTTGATAAACTGAATTTTTTTTAA | |
| | AAATCCATTTACCCTACAGGTTTGCATTTGTTTGCTGAAATTTACCTTTTTTTAG | |
| | TTATATATATGTGTGTGTGTGTGTGTAATTTCTGCCAATAAATTCTAAATTACAA | |
| | AGGTAAGAGAAAACCTAGTACATTACTAAATATATAAAGTATATGTTCTGATTAT | |
| | GTATACTTGTTCTAGTGTCAAGTCTTTTTAAGTGGGTTTTTAAAAGTTTGTTATT | |
| | GGACTTGAATGGATTTTTGAGACTAGGTTAATTATTTTTGAGGTCTTATCCTAA | |
| | AAGGCATCTAAGGTACATGAATGGAGTATGGTGATTTTATAACATTTTTTATCA | |
| | GAATGGAAAAAGAACTGTTTAAAAGTTTGATACTTTTAAATAGTTGGTTTTTTT | |
| | GCTTACTCTGGTAATGATTTTCTACAAATACATAATAAATTGTTTTTTTGAGTCT | |
| | ATATTCTGTATGCAGTTGAATATCCATTACTTATTCTGCTGTGCTTTAATAGAAT | |
| | GGAATGTTTACAGGCCCTTAAAATATTATTTTTAAAAAACCTTCTGAAGATACA | |
| | TACCAAAGTTTTTCCAAGAAGATTTTATAATCAATTTAATAATGTAAGGTTTATC | |
| | AGATTCTATAATAGTATAGTTATTAAGGCAATTTTATGTTAGAGACTATTTTGTA | |
| | ATGTAGTGAGTGGTACCTTTATAAGAAAAGTGACTGCCAATATATTTTTATAGC | |
| | TGATCTTTATAAATTCTAATGTTGAGTTTTTAATGATTATTTTAAATGTTTATATA | |
| | GTTTAGTAAAATTTGCATCTCAAAGTATCATTTTTATATTATGGGACGTTTTCAG | |
| | ATTGGCTAATATTTGCATTGTAAATTTTGTATGCAGTTTATCTAAAATTCAAAAA | |
| | TACTGTCAGTACACCAGCGTTTAACATCTATATTCCAATTTGTATACAGTTTAAA | |
| | ATTGTACTGCAAAACTATTGTGTGCTCTTACACAGTATGCATCATATTGTTGTCT | |
| | GTGAAATTAAAGGACATTTGATAGTCTACTGAATGTAAAATATAATGCTTGGTA | |
| | TATGAATGATAA | |
| 8 | MASTDYSTYSQAAAQQGYSAYTAQPTQGYAQTTQAYGQQSYGTYGQPTDVSY | Predicted Type 2 |
| | TQAQTTATYGQTAYATSYGQPPTGYTTPTAPQAYSQPVQGYGTGAYDTTTATVTT | EWSR1-ATF1 |
| | TQASYAAQSAYGTQPAYPAYGQQPAATAPTRPQDGNKPTETSQPQSSTGGYNQPS | fusion amino acid |
| | LGYGQSNYSYPQVPGSYPMQPVTAPPSYPPTSYSSTQPTSYDQSSYSQQNTYGQP | mRNA sequence |
| | SSYGQQSSYGQQSSYGQQPPTSYPPQTGSYSQAPSQYSQQSSSYGQQIAIAPNGA | (426 aa) |
| | LQLASPGTDGVQGLQTLTMTNSGSTQQGTTILQYAQTSDGQQILVPSNQVVVQT | |
| | ASGDMQTYQIRTTPSATSLPQTVVMTSPVTLTSQTTKTDDPQLKREIRLMKNREA | |
| | ARECRRKKKEYVKCLENRVAVLENQNKTLIEELKTLKDLYSNKSV | |
| 9 | TGGCCATTGCATACGTTGTATCCATATCATAATATGTACATTTATATTGGCTCATG | pGBI-2001 |
| | TCCAACATTACCGCCATGTTGACATTGATTATTGACTAGTTATTAATAGTAATCA | (expression plasmid |
| | ATTACGGGGTCATTAGTTCATAGCCCATATATGGAGTTCCGCGTTACATAACTTA | expressing the rare |
| | CGGTAAATGGCCCGCCTGGCTGACCGCCCAACGACCCCCGCCCATTGACGTC | fusion protein) |
| | AATAATGACGTATGTTCCCATAGTAACGCCAATAGGGACTTTCCATTGACGTCA | |
| | ATGGGTGGAGTATTTACGGTAAACTGCCCACTTGGCAGTACATCAAGTGTATC | |
| | ATATGCCAAGTACGCCCCCTATTGACGTCAATGACGGTAAATGGCCCGCCTGG | |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACGGTATCGATAAGCTTGATATCGAATTCGCCACCATGGCGTCC | |
| | ACGGATTACAGTACCTATAGCCAAGCTGCAGCGCAGCAGGGCTACAGTGCTTA | |
| | CACCGCCCAGCCCACTCAAGGATATGCACAGACCACCCAGGCATATGGGCAA | |
| | CAAAGCTATGGAACCTATGGACAGCCCACTGATGTCAGCTATACCCAGGCTCA | |
| | GACCACTGCAACCTATGGGCAGACCGCCTATGCAACTTCTTATGGACAGCCTC | |
| | CCACTGGTTATACTACTCCAACTGCCCCCCAGGCATACAGCCAGCCTGTCCAG | |
| | GGGTATGGCACTGGTGCTTATGATACCACCACTGCTACAGTCACCACCACCCA | |
| | GGCCTCCTATGCAGCTCAGTCTGCATATGGCACTCAGCCTGCTTATCCAGCCTA | |
| | TGGGCAGCAGCCAGCAGCCACTGCACCTACAAGACCGCAGGATGGAAACAA | |
| | GCCCACTGAGACTAGTCAACCTCAATCTAGCACAGGGGGTTACAACCAGCCC | |
| | AGCCTAGGATATGGACAGAGTAACTACAGTTATCCCCAGGTACCTGGGAGCTA | |
| | CCCCATGCAGCCAGTCACTGCACCTCCATCCTACCCTCCTACCAGCTATTCCTC | |
| | TACACAGCCGACTAGTTATGATCAGAGCAGTTACTCTCAGCAGAACACCTATG | |
| | GGCAACCGAGCAGCTATGGACAGCAGAGTAGCTATGGTCAACAAAGCAGCTA | |
| | TGGGCAGCAGCCTCCCACTAGTTACCCACCCCAAACTGGATCCTACAGCCAA | |
| | GCTCCAAGTCAATATAGCCAACAGAGCAGCAGCTACGGGCAGCAGAGTTCAT | |
| | TCCGACAGGACCACCCCAGTAGCATGGGTGTTTATGGGCAGGAGTCTGGAGG | |
| | ATTTTCCGGACCAGGAGAGAACCGGAGCATGAGTGGCCCTGATAACCGGGGC | |
| | AGGGGAAGAGGGGGATTTGATCGTGGAGGCATGAGCAGAGGTGGGCGGGGA | |
| | GGAGGACGCGGTGGAATGGGCAGCGCTGGAGAGCGAGGTGGCTTCAATAAG | |
| | CCTGGTGGACCCATGGATGAAGGACCAGATCTTGATCTAGGCCCACCTGTAGA | |
| | TCCAGATGAAGACTCTGACAACAGTGCAATTTATGTACAAGGATTAAATGACA | |
| | GTGTGACTCTAGATGATCTGGCAGACTTCTTTAAGCAGTGTGGGGTTGTTAAG | |
| | ATGAACAAGAGAACTGGGCAACCCATGATCCACATCTACCTGGACAAGGAAA | |
| | CAGGAAAGCCCAAAGGCGATGCCACAGTGTCCTATGAAGACCCACCCACTGC | |
| | CAAGGCTGCCGTGGAATGGTTTGATGCTGCATCAGGAGATATGCAAACATATC | |
| | AGATCCGAACTACACCTTCAGCTACTTCTCTGCCACAAACTGTGGTGATGACA | |
| | TCTCCTGTGACTCTCACCTCTCAGACAACTAAGACAGATGACCCCCAATTGAA | |
| | AAGAGAAATAAGGTTAATGAAAAACAGAGAAGCTGCTCGAGAATGTCGCAG | |
| | AAAGAAGAAAGAATATGTGAAATGCCTGGAAAACCGAGTTGCAGTCCTGGA | |
| | AAATCAAAATAAAACTCTAATAGAAGAGTTAAAAACTTTGAAGGATCTTTATT | |
| | CCAATAAAAGTGTTTGAGCGGCCGCGGATCCAGATCTTTTTCCCTCTGCCAAA | |
| | AATTATGGGGACATCATGAAGCCCCTTGAGCATCTGACTTCTGGCTAATAAAG | |
| | GAAATTTATTTTCATTGCAATAGTGTGTTGGAATTTTTTGTGTCTCTCACTCGG | |
| | AAGGACATATGGGAGGGCAAATCATTTAAAACATCAGAATGAGTATTTGGTTT | |
| | AGAGTTTGGCAACATATGCCCATTCTTCCGCTTCCTCGCTCACTGACTCGCTG | |
| | CGCTCGGTCGTTCGGCTGCGGCGAGCGGTATCAGCTCACTCAAAGGCGGTAA | |
| | TACGGTTATCCACAGAATCAGGGGATAACGCAGGAAAGAACATGTGAGCAAA | |
| | AGGCCAGCAAAAGGCCAGGAACCGTAAAAAGGCCGCGTTGCTGGCGTTTTT | |
| | CCATAGGCTCCGCCCCCCTGACGAGCATCACAAAAATCGACGCTCAAGTCAG | |
| | AGGTGGCGAAACCCGACAGGACTATAAAGATACCAGGCGTTTCCCCCTGGAA | |
| | GCTCCCTCGTGCGCTCTCCTGTTCCGACCCTGCCGCTTACCGGATACCTGTCC | |
| | GCCTTTCTCCCTTCGGGAAGCGTGGCGCTTTCTCATAGCTCACGCTGTAGGTA | |
| | TCTCAGTTCGGTGTAGGTCGTTCGCTCCAAGCTGGGCTGTGTGCACGAACCC | |
| | CCCGTTCAGCCCGACCGCTGCGCCTTATCCGGTAACTATCGTCTTGAGTCCAA | |
| | CCCGGTAAGACACGACTTATCGCCACTGGCAGCAGCCACTGGTAACAGGATT | |
| | AGCAGAGCGAGGTATGTAGGCGGTGCTACAGAGTTCTTGAAGTGGTGGCCTA | |
| | ACTACGGCTACACTAGAAGAACAGTATTTGGTATCTGCGCTCTGCTGAAGCCA | |
| | GTTACCTTCGGAAAAAGAGTTGGTAGCTCTTGATCCGGCAAACAAACCACCG | |
| | CTGGTAGCGGTGGTTTTTTTGTTTGCAAGCAGCAGATTACGCGCAGAAAAAA | |
| | AGGATCTCAAGAAGATCCTTTGATCTTTTCTACGGGGTCTGACGCTCAGTGGA | |
| | ACGAAAACTCACGTTAAGGGATTTTGGTCATGAGATTATCAAAAAGGATCTTC | |
| | ACCTAGATCCTTTTAAATTAAAAATGAAGTTTTAAATCAATCTAAAGTATATATG | |
| | AGTAAACTTGGTCTGACAGTTACCAATGCTTAATCAGTGAGGCACCTATCTCA | |
| | GCGATCTGTCTATTTCGTTCATCCATAGTTGCCTGACTCGGGGGGGGGGGGCG | |
| | CTGAGGTCTGCCTCGTGAAGAAGGTGTTGCTGACTCATACCAGGCCTGAATC | |
| | GCCCCATCATCCAGCCAGAAAGTGAGGGAGCCACGGTTGATGAGAGCTTTGT | |
| | TGTAGGTGGACCAGTTGGTGATTTTGAACTTTTGCTTTGCCACGGAACGGTCT | |
| | GCGTTGTCGGGAAGATGCGTGATCTGATCCTTCAACTCAGCAAAAGTTCGATT | |
| | TATTCAACAAAGCCGCCGTCCCGTCAAGTCAGCGTAATGCTCTGCCAGTGTTA | |
| | CAACCAATTAACCAATTCTGATTAGAAAAACTCATCGAGCATCAAATGAAACT | |
| | GCAATTTATTCATATCAGGATTATCAATACCATATTTTTGAAAAAGCCGTTTCTG | |
| | TAATGAAGGAGAAAACTCACCGAGGCAGTTCCATAGGATGGCAAGATCCTGG | |
| | TATCGGTCTGCGATTCCGACTCGTCCAACATCAATACAACCTATTAATTTCCCC | |
| | TCGTCAAAAATAAGGTTATCAAGTGAGAAATCACCATGAGTGACGACTGAAT | |
| | CCGGTGAGAATGGCAAAAGCTTATGCATTTCTTTCCAGACTTGTTCAACAGGC | |
| | CAGCCATTACGCTCGTCATCAAAATCACTCGCATCAACCAAACCGTTATTCATT | |
| | CGTGATTGCGCCTGAGCGAGACGAAATACGCGATCGCTGTTAAAAGGACAAT | |
| | TACAAACAGGAATCGAATGCAACCGGCGCAGGAACACTGCCAGCGCATCAAC | |
| | AATATTTTCACCTGAATCAGGATATTCTTCTAATACCTGGAATGCTGTTTTCCCG | |
| | GGGATCGCAGTGGTGAGTAACCATGCATCATCAGGAGTACGGATAAAATGCTT | |
| | GATGGTCGGAAGAGGCATAAATTCCGTCAGCCAGTTTAGTCTGACCATCTCAT | |
| | CTGTAACATCATTGGCAACGCTACCTTTGCCATGTTTCAGAAACAACTCTGGC | |
| | GCATCGGGCTTCCCATACAATCGATAGATTGTCGCACCTGATTGCCCGACATTA | |
| | TCGCGAGCCCATTTATACCCATATAAATCAGCATCCATGTTGGAATTTAATCGC | |
| | GGCCTCGAGCAAGACGTTTCCCGTTGAATATGGCTCATAACACCCCTTGTATT | |
| | ACTGTTTATGTAAGCAGACAGTTTTATTGTTCATGATGATATATTTTTATCTTGT | |
| | GCAATGTAACATCAGAGATTTTGAGACACAACGTGGCTTTCCCCCCCCCCCCA | |
| | TTATTGAAGCATTTATCAGGGTTATTGTCTCATGAGCGGATACATATTTGAATGT | |
| | ATTTAGAAAAATAAACAAATAGGGGTTCCGCGCACATTTCCCCGAAAAGTGCC | |
| | ACCTGACGTCTAAGAAACCATTATTATCATGACATTAACCTATAAAAATAGGCG | |
| | TATCACGAGGCCCTTTCGTCTCGCGCGTTTCGGTGATGACGGTGAAAACCTCT | |
| | GACACATGCAGCTCCCGGAGACGGTCACAGCTTGTCTGTAAGCGGATGCCGG | |
| | GAGCAGACAAGCCCGTCAGGGCGCGTCAGCGGGTGTTGGCGGGTGTCGGGG | |
| | CTGGCTTAACTATGCGGCATCAGAGCAGATTGTACTGAGAGTGCACCATATGC | |
| | GGTGTGAAATACCGCACAGATGCGTAAGGAGAAAATACCGCATCAGATTGGC | |
| | TAT | |
| 10 | TGGCCATTGCATACGTTGTATCCATATCATAATATGTACATTTATATTGGCTCATG | pGBI-2002 |
| | TCCAACATTACCGCCATGTTGACATTGATTATTGACTAGTTATTAATAGTAATCA | (expression plasmid |
| | ATTACGGGGTCATTAGTTCATAGCCCATATATGGAGTTCCGCGTTACATAACTTA | expressing the rare |
| | CGGTAAATGGCCCGCCTGGCTGACCGCCCAACGACCCCCGCCCATTGACGTC | fusion protein with |
| | AATAATGACGTATGTTCCCATAGTAACGCCAATAGGGACTTTCCATTGACGTCA | Strep tag at the |
| | ATGGGTGGAGTATTTACGGTAAACTGCCCACTTGGCAGTACATCAAGTGTATC | amino terminus) |
| | ATATGCCAAGTACGCCCCCTATTGACGTCAATGACGGTAAATGGCCCGCCTGG | |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACGGTATCGATAAGCTTGATATCGAATTCGCCACCATGTGGAGC | |
| | CATCCTCAGTTTGAGAAAGGTTCTATGGCGTCCACGGATTACAGTACCTATAG | |
| | CCAAGCTGCAGCGCAGCAGGGCTACAGTGCTTACACCGCCCAGCCCACTCAA | |
| | GGATATGCACAGACCACCCAGGCATATGGGCAACAAAGCTATGGAACCTATGG | |
| | ACAGCCCACTGATGTCAGCTATACCCAGGCTCAGACCACTGCAACCTATGGGC | |
| | AGACCGCCTATGCAACTTCTTATGGACAGCCTCCCACTGGTTATACTACTCCA | |
| | ACTGCCCCCCAGGCATACAGCCAGCCTGTCCAGGGGTATGGCACTGGTGCTTA | |
| | TGATACCACCACTGCTACAGTCACCACCACCCAGGCCTCCTATGCAGCTCAGT | |
| | CTGCATATGGCACTCAGCCTGCTTATCCAGCCTATGGGCAGCAGCCAGCAGCC | |
| | ACTGCACCTACAAGACCGCAGGATGGAAACAAGCCCACTGAGACTAGTCAA | |
| | CCTCAATCTAGCACAGGGGGTTACAACCAGCCCAGCCTAGGATATGGACAGA | |
| | GTAACTACAGTTATCCCCAGGTACCTGGGAGCTACCCCATGCAGCCAGTCACT | |
| | GCACCTCCATCCTACCCTCCTACCAGCTATTCCTCTACACAGCCGACTAGTTAT | |
| | GATCAGAGCAGTTACTCTCAGCAGAACACCTATGGGCAACCGAGCAGCTATG | |
| | GACAGCAGAGTAGCTATGGTCAACAAAGCAGCTATGGGCAGCAGCCTCCCAC | |
| | TAGTTACCCACCCCAAACTGGATCCTACAGCCAAGCTCCAAGTCAATATAGCC | |
| | AACAGAGCAGCAGCTACGGGCAGCAGAGTTCATTCCGACAGGACCACCCCA | |
| | GTAGCATGGGTGTTTATGGGCAGGAGTCTGGAGGATTTTCCGGACCAGGAGA | |
| | GAACCGGAGCATGAGTGGCCCTGATAACCGGGGCAGGGGAAGAGGGGGATT | |
| | TGATCGTGGAGGCATGAGCAGAGGTGGGCGGGGAGGAGGACGCGGTGGAAT | |
| | GGGCAGCGCTGGAGAGCGAGGTGGCTTCAATAAGCCTGGTGGACCCATGGAT | |
| | GAAGGACCAGATCTTGATCTAGGCCCACCTGTAGATCCAGATGAAGACTCTG | |
| | ACAACAGTGCAATTTATGTACAAGGATTAAATGACAGTGTGACTCTAGATGAT | |
| | CTGGCAGACTTCTTTAAGCAGTGTGGGGTTGTTAAGATGAACAAGAGAACTG | |
| | GGCAACCCATGATCCACATCTACCTGGACAAGGAAACAGGAAAGCCCAAAG | |
| | GCGATGCCACAGTGTCCTATGAAGACCCACCCACTGCCAAGGCTGCCGTGGA | |
| | ATGGTTTGATGCTGCATCAGGAGATATGCAAACATATCAGATCCGAACTACAC | |
| | CTTCAGCTACTTCTCTGCCACAAACTGTGGTGATGACATCTCCTGTGACTCTC | |
| | ACCTCTCAGACAACTAAGACAGATGACCCCCAATTGAAAAGAGAAATAAGGT | |
| | TAATGAAAAACAGAGAAGCTGCTCGAGAATGTCGCAGAAAGAAGAAAGAAT | |
| | ATGTGAAATGCCTGGAAAACCGAGTTGCAGTCCTGGAAAATCAAAATAAAAC | |
| | TCTAATAGAAGAGTTAAAAACTTTGAAGGATCTTTATTCCAATAAAAGTGTTT | |
| | GAGCGGCCGCGGATCCAGATCTTTTTCCCTCTGCCAAAAATTATGGGGACATC | |
| | ATGAAGCCCCTTGAGCATCTGACTTCTGGCTAATAAAGGAAATTTATTTTCATT | |
| | GCAATAGTGTGTTGGAATTTTTTGTGTCTCTCACTCGGAAGGACATATGGGAG | |
| | GGCAAATCATTTAAAACATCAGAATGAGTATTTGGTTTAGAGTTTGGCAACAT | |
| | ATGCCCATTCTTCCGCTTCCTCGCTCACTGACTCGCTGCGCTCGGTCGTTCGG | |
| | CTGCGGCGAGCGGTATCAGCTCACTCAAAGGCGGTAATACGGTTATCCACAG | |
| | AATCAGGGGATAACGCAGGAAAGAACATGTGAGCAAAAGGCCAGCAAAAGG | |
| | CCAGGAACCGTAAAAAGGCCGCGTTGCTGGCGTTTTTCCATAGGCTCCGCCC | |
| | CCCTGACGAGCATCACAAAAATCGACGCTCAAGTCAGAGGTGGCGAAACCC | |
| | GACAGGACTATAAAGATACCAGGCGTTTCCCCCTGGAAGCTCCCTCGTGCGCT | |
| | CTCCTGTTCCGACCCTGCCGCTTACCGGATACCTGTCCGCCTTTCTCCCTTCGG | |
| | GAAGCGTGGCGCTTTCTCATAGCTCACGCTGTAGGTATCTCAGTTCGGTGTAG | |
| | GTCGTTCGCTCCAAGCTGGGCTGTGTGCACGAACCCCCCGTTCAGCCCGACC | |
| | GCTGCGCCTTATCCGGTAACTATCGTCTTGAGTCCAACCCGGTAAGACACGAC | |
| | TTATCGCCACTGGCAGCAGCCACTGGTAACAGGATTAGCAGAGCGAGGTATG | |
| | TAGGCGGTGCTACAGAGTTCTTGAAGTGGTGGCCTAACTACGGCTACACTAG | |
| | AAGAACAGTATTTGGTATCTGCGCTCTGCTGAAGCCAGTTACCTTCGGAAAAA | |
| | GAGTTGGTAGCTCTTGATCCGGCAAACAAACCACCGCTGGTAGCGGTGGTTT | |
| | TTTTGTTTGCAAGCAGCAGATTACGCGCAGAAAAAAAGGATCTCAAGAAGAT | |
| | CCTTTGATCTTTTCTACGGGGTCTGACGCTCAGTGGAACGAAAACTCACGTTA | |
| | AGGGATTTTGGTCATGAGATTATCAAAAAGGATCTTCACCTAGATCCTTTTAAA | |
| | TTAAAAATGAAGTTTTAAATCAATCTAAAGTATATATGAGTAAACTTGGTCTGA | |
| | CAGTTACCAATGCTTAATCAGTGAGGCACCTATCTCAGCGATCTGTCTATTTCG | |
| | TTCATCCATAGTTGCCTGACTCGGGGGGGGGGGGCGCTGAGGTCTGCCTCGT | |
| | GAAGAAGGTGTTGCTGACTCATACCAGGCCTGAATCGCCCCATCATCCAGCCA | |
| | GAAAGTGAGGGAGCCACGGTTGATGAGAGCTTTGTTGTAGGTGGACCAGTTG | |
| | GTGATTTTGAACTTTTGCTTTGCCACGGAACGGTCTGCGTTGTCGGGAAGATG | |
| | CGTGATCTGATCCTTCAACTCAGCAAAAGTTCGATTTATTCAACAAAGCCGCC | |
| | GTCCCGTCAAGTCAGCGTAATGCTCTGCCAGTGTTACAACCAATTAACCAATT | |
| | CTGATTAGAAAAACTCATCGAGCATCAAATGAAACTGCAATTTATTCATATCAG | |
| | GATTATCAATACCATATTTTTGAAAAAGCCGTTTCTGTAATGAAGGAGAAAAC | |
| | TCACCGAGGCAGTTCCATAGGATGGCAAGATCCTGGTATCGGTCTGCGATTCC | |
| | GACTCGTCCAACATCAATACAACCTATTAATTTCCCCTCGTCAAAAATAAGGTT | |
| | ATCAAGTGAGAAATCACCATGAGTGACGACTGAATCCGGTGAGAATGGCAAA | |
| | AGCTTATGCATTTCTTTCCAGACTTGTTCAACAGGCCAGCCATTACGCTCGTC | |
| | ATCAAAATCACTCGCATCAACCAAACCGTTATTCATTCGTGATTGCGCCTGAG | |
| | CGAGACGAAATACGCGATCGCTGTTAAAAGGACAATTACAAACAGGAATCGA | |
| | ATGCAACCGGCGCAGGAACACTGCCAGCGCATCAACAATATTTTCACCTGAAT | |
| | CAGGATATTCTTCTAATACCTGGAATGCTGTTTTCCCGGGGATCGCAGTGGTGA | |
| | GTAACCATGCATCATCAGGAGTACGGATAAAATGCTTGATGGTCGGAAGAGGC | |
| | ATAAATTCCGTCAGCCAGTTTAGTCTGACCATCTCATCTGTAACATCATTGGCA | |
| | ACGCTACCTTTGCCATGTTTCAGAAACAACTCTGGCGCATCGGGCTTCCCATA | |
| | CAATCGATAGATTGTCGCACCTGATTGCCCGACATTATCGCGAGCCCATTTATA | |
| | CCCATATAAATCAGCATCCATGTTGGAATTTAATCGCGGCCTCGAGCAAGACG | |
| | TTTCCCGTTGAATATGGCTCATAACACCCCTTGTATTACTGTTTATGTAAGCAG | |
| | ACAGTTTTATTGTTCATGATGATATATTTTTATCTTGTGCAATGTAACATCAGAG | |
| | ATTTTGAGACACAACGTGGCTTTCCCCCCCCCCCCATTATTGAAGCATTTATCA | |
| | GGGTTATTGTCTCATGAGCGGATACATATTTGAATGTATTTAGAAAAATAAACA | |
| | AATAGGGGTTCCGCGCACATTTCCCCGAAAAGTGCCACCTGACGTCTAAGAA | |
| | ACCATTATTATCATGACATTAACCTATAAAAATAGGCGTATCACGAGGCCCTTT | |
| | CGTCTCGCGCGTTTCGGTGATGACGGTGAAAACCTCTGACACATGCAGCTCC | |
| | CGGAGACGGTCACAGCTTGTCTGTAAGCGGATGCCGGGAGCAGACAAGCCC | |
| | GTCAGGGCGCGTCAGCGGGTGTTGGCGGGTGTCGGGGCTGGCTTAACTATGC | |
| | GGCATCAGAGCAGATTGTACTGAGAGTGCACCATATGCGGTGTGAAATACCGC | |
| | ACAGATGCGTAAGGAGAAAATACCGCATCAGATTGGCTAT | |
| 11 | TGGCCATTGCATACGTTGTATCCATATCATAATATGTACATTTATATTGGCTCATG | pGBI-2003 (bi- |
| | TCCAACATTACCGCCATGTTGACATTGATTATTGACTAGTTATTAATAGTAATCA | shRNA construct #1 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTTTCTTCCCCATTAGGGATTATGCTGAATTTGTATGGTTT | |
| | ATAGTTGTTAGAGTTTGAGGTGTTAATTCTAATTATCTATTTCAAATTTAG | |
| | CAGGAAAAAAGAGAACATCACCTTGTAAAACTGAAGATTGTGACCAGTC | |
| | AGAATAATGTCGTGGAATGGTTTGATGCTGCATAGATATGTGCATCTATGC | |
| | AGCATCAAACCATTCCACGCATTATGGTGACAGCTGCCTCGGGAAGCCA | |
| | AGTTGGGCTTTAAAGTGCAGGGCCTGCTGATGTTGAGTGCTTTTTGTTC | |
| | CGTGTCATGCAGAGATGCTTGATAGTGAAGTAGATTAGCATCTATGCAGC | |
| | ATCAAACCATTCCACGCATAAGAAGTTATGTATTCATCCAATAATTCAAGC | |
| | CAAGCAAGTATATAGGTGTTTTAATAGTTTTTGTTTTGGGGATTGTGACC | |
| | AGAAGATTTTGAAAATTAAATATTACTGAAGATTTCGACTTCCACTGTTAA | |
| | ATGTACAAGATACATGAAATATTAAAGAAAATGTGTAACTTTTTGTGTAAA | |
| | TACATCTTGTGCGGCCGCGGATCCAGATCTTTTTCCCTCTGCCAAAAATTATG | |
| | GGGACATCATGAAGCCCCTTGAGCATCTGACTTCTGGCTAATAAAGGAAATTT | |
| | ATTTTCATTGCAATAGTGTGTTGGAATTTTTTGTGTCTCTCACTCGGAAGGACA | |
| | TATGGGAGGGCAAATCATTTAAAACATCAGAATGAGTATTTGGTTTAGAGTTT | |
| | GGCAACATATGCCCATTCTTCCGCTTCCTCGCTCACTGACTCGCTGCGCTCGG | |
| | TCGTTCGGCTGCGGCGAGCGGTATCAGCTCACTCAAAGGCGGTAATACGGTTA | |
| | TCCACAGAATCAGGGGATAACGCAGGAAAGAACATGTGAGCAAAAGGCCAG | |
| | CAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCG | |
| | AAACCCGACAGGACTATAAAGATACCAGGCGTTTCCCCCTGGAAGCTCCCTC | |
| | GTGCGCTCTCCTGTTCCGACCCTGCCGCTTACCGGATACCTGTCCGCCTTTCTC | |
| | CCTTCGGGAAGCGTGGCGCTTTCTCATAGCTCACGCTGTAGGTATCTCAGTTC | |
| | GGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCG | |
| | AGGTATGTAGGCGGTGCTACAGAGTTCTTGAAGTGGTGGCCTAACTACGGCTA | |
| | CACTAGAAGAACAGTATTTGGTATCTGCGCTCTGCTGAAGCCAGTTACCTTCG | |
| | GAAAAAGAGTTGGTAGCTCTTGATCCGGCAAACAAACCACCGCTGGTAGCGG | |
| | TGGTTTTTTTGTTTGCAAGCAGCAGATTACGCGCAGAAAAAAAGGATCTCAA | |
| | GAAGATCCTTTGATCTTTTCTACGGGGTCTGACGCTCAGTGGAACGAAAACTC | |
| | ACGTTAAGGGATTTTGGTCATGAGATTATCAAAAAGGATCTTCACCTAGATCCT | |
| | TTTAAATTAAAAATGAAGTTTTAAATCAATCTAAAGTATATATGAGTAAACTTG | |
| | GTCTGACAGTTACCAATGCTTAATCAGTGAGGCACCTATCTCAGCGATCTGTC | |
| | TATTTCGTTCATCCATAGTTGCCTGACTCGGGGGGGGGGGGCGCTGAGGTCTG | |
| | CCTCGTGAAGAAGGTGTTGCTGACTCATACCAGGCCTGAATCGCCCCATCATC | |
| | CAGCCAGAAAGTGAGGGAGCCACGGTTGATGAGAGCTTTGTTGTAGGTGGAC | |
| | CAGTTGGTGATTTTGAACTTTTGCTTTGCCACGGAACGGTCTGCGTTGTCGGG | |
| | AAGATGCGTGATCTGATCCTTCAACTCAGCAAAAGTTCGATTTATTCAACAAA | |
| | GCCGCCGTCCCGTCAAGTCAGCGTAATGCTCTGCCAGTGTTACAACCAATTAA | |
| | CCAATTCTGATTAGAAAAACTCATCGAGCATCAAATGAAACTGCAATTTATTC | |
| | ATATCAGGATTATCAATACCATATTTTTGAAAAAGCCGTTTCTGTAATGAAGGA | |
| | GAAAACTCACCGAGGCAGTTCCATAGGATGGCAAGATCCTGGTATCGGTCTG | |
| | CGATTCCGACTCGTCCAACATCAATACAACCTATTAATTTCCCCTCGTCAAAAA | |
| | TAAGGTTATCAAGTGAGAAATCACCATGAGTGACGACTGAATCCGGTGAGAA | |
| | TGGCAAAAGCTTATGCATTTCTTTCCAGACTTGTTCAACAGGCCAGCCATTAC | |
| | GCTCGTCATCAAAATCACTCGCATCAACCAAACCGTTATTCATTCGTGATTGCG | |
| | CCTGAGCGAGACGAAATACGCGATCGCTGTTAAAAGGACAATTACAAACAGG | |
| | AATCGAATGCAACCGGCGCAGGAACACTGCCAGCGCATCAACAATATTTTCA | |
| | CCTGAATCAGGATATTCTTCTAATACCTGGAATGCTGTTTTCCCGGGGATCGCA | |
| | GTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACAT | |
| | CATTGGCAACGCTACCTTTGCCATGTTTCAGAAACAACTCTGGCGCATCGGGC | |
| | TTCCCATACAATCGATAGATTGTCGCACCTGATTGCCCGACATTATCGCGAGCC | |
| | CATTTATACCCATATAAATCAGCATCCATGTTGGAATTTAATCGCGGCCTCGAG | |
| | CAAGACGTTTCCCGTTGAATATGGCTCATAACACCCCTTGTATTACTGTTTATG | |
| | TAAGCAGACAGTTTTATTGTTCATGATGATATATTTTTATCTTGTGCAATGTAAC | |
| | ATCAGAGATTTTGAGACACAACGTGGCTTTCCCCCCCCCCCCATTATTGAAGC | |
| | ATTTATCAGGGTTATTGTCTCATGAGCGGATACATATTTGAATGTATTTAGAAAA | |
| | ATAAACAAATAGGGGTTCCGCGCACATTTCCCCGAAAAGTGCCACCTGACGT | |
| | CTAAGAAACCATTATTATCATGACATTAACCTATAAAAATAGGCGTATCACGAG | |
| | GCCCTTTCGTCTCGCGCGTTTCGGTGATGACGGTGAAAACCTCTGACACATGC | |
| | AGCTCCCGGAGACGGTCACAGCTTGTCTGTAAGCGGATGCCGGGAGCAGACA | |
| | AGCCCGTCAGGGCGCGTCAGCGGGTGTTGGCGGGTGTCGGGGCTGGCTTAAC | |
| | TATGCGGCATCAGAGCAGATTGTACTGAGAGTGCACCATATGCGGTGTGAAAT | |
| | ACCGCACAGATGCGTAAGGAGAAAATACCGCATCAGATTGGCTAT | |
| 12 | TGGCCATTGCATACGTTGTATCCATATCATAATATGTACATTTATATTGGCTCATG | pGBI-2004 (bi- |
| | TCCAACATTACCGCCATGTTGACATTGATTATTGACTAGTTATTAATAGTAATCA | shRNA construct #2 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTTTCTTCCCCATTAGGGATTATGCTGAATTTGTATGGTTT | |
| | ATAGTTGTTAGAGTTTGAGGTGTTAATTCTAATTATCTATTTCAAATTTAG | |
| | CAGGAAAAAAGAGAACATCACCTTGTAAAACTGAAGATTGTGACCAGTC | |
| | AGAATAATGTCCGTGGAATGGTTTGATGCTGCAAGATATGTGCATCTTGC | |
| | AGCATCAAACCATTCCACGGCATTATGGTGACAGCTGCCTCGGGAAGCC | |
| | AAGTTGGGCTTTAAAGTGCAGGGCCTGCTGATGTTGAGTGCTTTTTGTT | |
| | CCCATGTAATGACCTGATGATACAAGTGAAGTAGATTAGCATCTTGCAGC | |
| | ATCAAACCATTCCACGGCATAAGAAGTTATGTATTCATCCAATAATTCAAG | |
| | CCAAGCAAGTATATAGGTGTTTTAATAGTTTTTGTTTTGGGGATTGTGAC | |
| | CAGAAGATTTTGAAAATTAAATATTACTGAAGATTTCGACTTCCACTGTTA | |
| | AATGTACAAGATACATGAAATATTAAAGAAAATGTGTAACTTTTTGTGTAA | |
| | ATACATCTTGTGCGGCCGCGGATCCAGATCTTTTTCCCTCTGCCAAAAATTAT | |
| | GGGGACATCATGAAGCCCCTTGAGCATCTGACTTCTGGCTAATAAAGGAAATT | |
| | TATTTTCATTGCAATAGTGTGTTGGAATTTTTTGTGTCTCTCACTCGGAAGGAC | |
| | ATATGGGAGGGCAAATCATTTAAAACATCAGAATGAGTATTTGGTTTAGAGTTT | |
| | GGCAACATATGCCCATTCTTCCGCTTCCTCGCTCACTGACTCGCTGCGCTCGG | |
| | TCGTTCGGCTGCGGCGAGCGGTATCAGCTCACTCAAAGGCGGTAATACGGTTA | |
| | TCCACAGAATCAGGGGATAACGCAGGAAAGAACATGTGAGCAAAAGGCCAG | |
| | CAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCG | |
| | AAACCCGACAGGACTATAAAGATACCAGGCGTTTCCCCCTGGAAGCTCCCTC | |
| | GTGCGCTCTCCTGTTCCGACCCTGCCGCTTACCGGATACCTGTCCGCCTTTCTC | |
| | CCTTCGGGAAGCGTGGCGCTTTCTCATAGCTCACGCTGTAGGTATCTCAGTTC | |
| | GGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCG | |
| | AGGTATGTAGGCGGTGCTACAGAGTTCTTGAAGTGGTGGCCTAACTACGGCTA | |
| | CACTAGAAGAACAGTATTTGGTATCTGCGCTCTGCTGAAGCCAGTTACCTTCG | |
| | GAAAAAGAGTTGGTAGCTCTTGATCCGGCAAACAAACCACCGCTGGTAGCGG | |
| | TGGTTTTTTTGTTTGCAAGCAGCAGATTACGCGCAGAAAAAAAGGATCTCAA | |
| | GAAGATCCTTTGATCTTTTCTACGGGGTCTGACGCTCAGTGGAACGAAAACTC | |
| | ACGTTAAGGGATTTTGGTCATGAGATTATCAAAAAGGATCTTCACCTAGATCCT | |
| | TTTAAATTAAAAATGAAGTTTTAAATCAATCTAAAGTATATATGAGTAAACTTG | |
| | GTCTGACAGTTACCAATGCTTAATCAGTGAGGCACCTATCTCAGCGATCTGTC | |
| | TATTTCGTTCATCCATAGTTGCCTGACTCGGGGGGGGGGGGCGCTGAGGTCTG | |
| | CCTCGTGAAGAAGGTGTTGCTGACTCATACCAGGCCTGAATCGCCCCATCATC | |
| | CAGCCAGAAAGTGAGGGAGCCACGGTTGATGAGAGCTTTGTTGTAGGTGGAC | |
| | CAGTTGGTGATTTTGAACTTTTGCTTTGCCACGGAACGGTCTGCGTTGTCGGG | |
| | AAGATGCGTGATCTGATCCTTCAACTCAGCAAAAGTTCGATTTATTCAACAAA | |
| | GCCGCCGTCCCGTCAAGTCAGCGTAATGCTCTGCCAGTGTTACAACCAATTAA | |
| | CCAATTCTGATTAGAAAAACTCATCGAGCATCAAATGAAACTGCAATTTATTC | |
| | ATATCAGGATTATCAATACCATATTTTTGAAAAAGCCGTTTCTGTAATGAAGGA | |
| | GAAAACTCACCGAGGCAGTTCCATAGGATGGCAAGATCCTGGTATCGGTCTG | |
| | CGATTCCGACTCGTCCAACATCAATACAACCTATTAATTTCCCCTCGTCAAAAA | |
| | TAAGGTTATCAAGTGAGAAATCACCATGAGTGACGACTGAATCCGGTGAGAA | |
| | TGGCAAAAGCTTATGCATTTCTTTCCAGACTTGTTCAACAGGCCAGCCATTAC | |
| | GCTCGTCATCAAAATCACTCGCATCAACCAAACCGTTATTCATTCGTGATTGCG | |
| | CCTGAGCGAGACGAAATACGCGATCGCTGTTAAAAGGACAATTACAAACAGG | |
| | AATCGAATGCAACCGGCGCAGGAACACTGCCAGCGCATCAACAATATTTTCA | |
| | CCTGAATCAGGATATTCTTCTAATACCTGGAATGCTGTTTTCCCGGGGATCGCA | |
| | GTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACAT | |
| | CATTGGCAACGCTACCTTTGCCATGTTTCAGAAACAACTCTGGCGCATCGGGC | |
| | TTCCCATACAATCGATAGATTGTCGCACCTGATTGCCCGACATTATCGCGAGCC | |
| | CATTTATACCCATATAAATCAGCATCCATGTTGGAATTTAATCGCGGCCTCGAG | |
| | CAAGACGTTTCCCGTTGAATATGGCTCATAACACCCCTTGTATTACTGTTTATG | |
| | TAAGCAGACAGTTTTATTGTTCATGATGATATATTTTTATCTTGTGCAATGTAAC | |
| | ATCAGAGATTTTGAGACACAACGTGGCTTTCCCCCCCCCCCCATTATTGAAGC | |
| | ATTTATCAGGGTTATTGTCTCATGAGCGGATACATATTTGAATGTATTTAGAAAA | |
| | ATAAACAAATAGGGGTTCCGCGCACATTTCCCCGAAAAGTGCCACCTGACGT | |
| | CTAAGAAACCATTATTATCATGACATTAACCTATAAAAATAGGCGTATCACGAG | |
| | GCCCTTTCGTCTCGCGCGTTTCGGTGATGACGGTGAAAACCTCTGACACATGC | |
| | AGCTCCCGGAGACGGTCACAGCTTGTCTGTAAGCGGATGCCGGGAGCAGACA | |
| | AGCCCGTCAGGGCGCGTCAGCGGGTGTTGGCGGGTGTCGGGGCTGGCTTAAC | |
| | TATGCGGCATCAGAGCAGATTGTACTGAGAGTGCACCATATGCGGTGTGAAAT | |
| | ACCGCACAGATGCGTAAGGAGAAAATACCGCATCAGATTGGCTAT | |
| 13 | TGGCCATTGCATACGTTGTATCCATATCATAATATGTACATTTATATTGGCTCATG | pGBI-2005 (bi- |
| | TCCAACATTACCGCCATGTTGACATTGATTATTGACTAGTTATTAATAGTAATCA | shRNA construct #3 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTTTCTTCCCCATTAGGGATTATGCTGAATTTGTATGGTTT | |
| | ATAGTTGTTAGAGTTTGAGGTGTTAATTCTAATTATCTATTTCAAATTTAG | |
| | CAGGAAAAAAGAGAACATCACCTTGTAAAACTGAAGATTGTGACCAGTC | |
| | AGAATAATGTGCCGTGGAATGGTTTGATGCTGCAGATATGTGCATCTGCA | |
| | GCATCAAACCATTCCACGGCCATTATGGTGACAGCTGCCTCGGGAAGCC | |
| | AAGTTGGGCTTTAAAGTGCAGGGCCTGCTGATGTTGAGTGCTTTTTGTT | |
| | CGCTATGTCATACATTGATGACGCAGTGAAGTAGATTAGCATCTGCAGCA | |
| | TCAAACCATTCCACGGCCATAAGAAGTTATGTATTCATCCAATAATTCAAG | |
| | CCAAGCAAGTATATAGGTGTTTTAATAGTTTTTGTTTTGGGGATTGTGAC | |
| | CAGAAGATTTTGAAAATTAAATATTACTGAAGATTTCGACTTCCACTGTTA | |
| | AATGTACAAGATACATGAAATATTAAAGAAAATGTGTAACTTTTTGTGTAA | |
| | ATACATCTTGTGCGGCCGCGGATCCAGATCTTTTTCCCTCTGCCAAAAATTAT | |
| | GGGGACATCATGAAGCCCCTTGAGCATCTGACTTCTGGCTAATAAAGGAAATT | |
| | TATTTTCATTGCAATAGTGTGTTGGAATTTTTTGTGTCTCTCACTCGGAAGGAC | |
| | ATATGGGAGGGCAAATCATTTAAAACATCAGAATGAGTATTTGGTTTAGAGTTT | |
| | GGCAACATATGCCCATTCTTCCGCTTCCTCGCTCACTGACTCGCTGCGCTCGG | |
| | TCGTTCGGCTGCGGCGAGCGGTATCAGCTCACTCAAAGGCGGTAATACGGTTA | |
| | TCCACAGAATCAGGGGATAACGCAGGAAAGAACATGTGAGCAAAAGGCCAG | |
| | CAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCG | |
| | AAACCCGACAGGACTATAAAGATACCAGGCGTTTCCCCCTGGAAGCTCCCTC | |
| | GTGCGCTCTCCTGTTCCGACCCTGCCGCTTACCGGATACCTGTCCGCCTTTCTC | |
| | CCTTCGGGAAGCGTGGCGCTTTCTCATAGCTCACGCTGTAGGTATCTCAGTTC | |
| | GGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCG | |
| | AGGTATGTAGGCGGTGCTACAGAGTTCTTGAAGTGGTGGCCTAACTACGGCTA | |
| | CACTAGAAGAACAGTATTTGGTATCTGCGCTCTGCTGAAGCCAGTTACCTTCG | |
| | GAAAAAGAGTTGGTAGCTCTTGATCCGGCAAACAAACCACCGCTGGTAGCGG | |
| | TGGTTTTTTTGTTTGCAAGCAGCAGATTACGCGCAGAAAAAAAGGATCTCAA | |
| | GAAGATCCTTTGATCTTTTCTACGGGGTCTGACGCTCAGTGGAACGAAAACTC | |
| | ACGTTAAGGGATTTTGGTCATGAGATTATCAAAAAGGATCTTCACCTAGATCCT | |
| | TTTAAATTAAAAATGAAGTTTTAAATCAATCTAAAGTATATATGAGTAAACTTG | |
| | GTCTGACAGTTACCAATGCTTAATCAGTGAGGCACCTATCTCAGCGATCTGTC | |
| | TATTTCGTTCATCCATAGTTGCCTGACTCGGGGGGGGGGGGCGCTGAGGTCTG | |
| | CCTCGTGAAGAAGGTGTTGCTGACTCATACCAGGCCTGAATCGCCCCATCATC | |
| | CAGCCAGAAAGTGAGGGAGCCACGGTTGATGAGAGCTTTGTTGTAGGTGGAC | |
| | CAGTTGGTGATTTTGAACTTTTGCTTTGCCACGGAACGGTCTGCGTTGTCGGG | |
| | AAGATGCGTGATCTGATCCTTCAACTCAGCAAAAGTTCGATTTATTCAACAAA | |
| | GCCGCCGTCCCGTCAAGTCAGCGTAATGCTCTGCCAGTGTTACAACCAATTAA | |
| | CCAATTCTGATTAGAAAAACTCATCGAGCATCAAATGAAACTGCAATTTATTC | |
| | ATATCAGGATTATCAATACCATATTTTTGAAAAAGCCGTTTCTGTAATGAAGGA | |
| | GAAAACTCACCGAGGCAGTTCCATAGGATGGCAAGATCCTGGTATCGGTCTG | |
| | CGATTCCGACTCGTCCAACATCAATACAACCTATTAATTTCCCCTCGTCAAAAA | |
| | TAAGGTTATCAAGTGAGAAATCACCATGAGTGACGACTGAATCCGGTGAGAA | |
| | TGGCAAAAGCTTATGCATTTCTTTCCAGACTTGTTCAACAGGCCAGCCATTAC | |
| | GCTCGTCATCAAAATCACTCGCATCAACCAAACCGTTATTCATTCGTGATTGCG | |
| | CCTGAGCGAGACGAAATACGCGATCGCTGTTAAAAGGACAATTACAAACAGG | |
| | AATCGAATGCAACCGGCGCAGGAACACTGCCAGCGCATCAACAATATTTTCA | |
| | CCTGAATCAGGATATTCTTCTAATACCTGGAATGCTGTTTTCCCGGGGATCGCA | |
| | GTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACAT | |
| | CATTGGCAACGCTACCTTTGCCATGTTTCAGAAACAACTCTGGCGCATCGGGC | |
| | TTCCCATACAATCGATAGATTGTCGCACCTGATTGCCCGACATTATCGCGAGCC | |
| | CATTTATACCCATATAAATCAGCATCCATGTTGGAATTTAATCGCGGCCTCGAG | |
| | CAAGACGTTTCCCGTTGAATATGGCTCATAACACCCCTTGTATTACTGTTTATG | |
| | TAAGCAGACAGTTTTATTGTTCATGATGATATATTTTTATCTTGTGCAATGTAAC | |
| | ATCAGAGATTTTGAGACACAACGTGGCTTTCCCCCCCCCCCCATTATTGAAGC | |
| | ATTTATCAGGGTTATTGTCTCATGAGCGGATACATATTTGAATGTATTTAGAAAA | |
| | ATAAACAAATAGGGGTTCCGCGCACATTTCCCCGAAAAGTGCCACCTGACGT | |
| | CTAAGAAACCATTATTATCATGACATTAACCTATAAAAATAGGCGTATCACGAG | |
| | GCCCTTTCGTCTCGCGCGTTTCGGTGATGACGGTGAAAACCTCTGACACATGC | |
| | AGCTCCCGGAGACGGTCACAGCTTGTCTGTAAGCGGATGCCGGGAGCAGACA | |
| | AGCCCGTCAGGGCGCGTCAGCGGGTGTTGGCGGGTGTCGGGGCTGGCTTAAC | |
| | TATGCGGCATCAGAGCAGATTGTACTGAGAGTGCACCATATGCGGTGTGAAAT | |
| | ACCGCACAGATGCGTAAGGAGAAAATACCGCATCAGATTGGCTAT | |
| 14 | TGGCCATTGCATACGTTGTATCCATATCATAATATGTACATTTATATTGGCTCATG | pGBI-2006 (bi- |
| | TCCAACATTACCGCCATGTTGACATTGATTATTGACTAGTTATTAATAGTAATCA | shRNA construct #4 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTTTCTTCCCCATTAGGGATTATGCTGAATTTGTATGGTTT | |
| | ATAGTTGTTAGAGTTTGAGGTGTTAATTCTAATTATCTATTTCAAATTTAG | |
| | CAGGAAAAAAGAGAACATCACCTTGTAAAACTGAAGATTGTGACCAGTC | |
| | AGAATAATGTTGCCGTGGAATGGTTTGATGCTGAGATATGTGCATCTCAG | |
| | CATCAAACCATTCCACGGCACATTATGGTGACAGCTGCCTCGGGAAGCC | |
| | AAGTTGGGCTTTAAAGTGCAGGGCCTGCTGATGTTGAGTGCTTTTTGTT | |
| | CTGATGTGTAACAATTTGACGATGAGTGAAGTAGATTAGCATCTCAGCAT | |
| | CAAACCATTCCACGGCACATAAGAAGTTATGTATTCATCCAATAATTCAAG | |
| | CCAAGCAAGTATATAGGTGTTTTAATAGTTTTTGTTTTGGGGATTGTGAC | |
| | CAGAAGATTTTGAAAATTAAATATTACTGAAGATTTCGACTTCCACTGTTA | |
| | AATGTACAAGATACATGAAATATTAAAGAAAATGTGTAACTTTTTGTGTAA | |
| | ATACATCTTGTGCGGCCGCGGATCCAGATCTTTTTCCCTCTGCCAAAAATTAT | |
| | GGGGACATCATGAAGCCCCTTGAGCATCTGACTTCTGGCTAATAAAGGAAATT | |
| | TATTTTCATTGCAATAGTGTGTTGGAATTTTTTGTGTCTCTCACTCGGAAGGAC | |
| | ATATGGGAGGGCAAATCATTTAAAACATCAGAATGAGTATTTGGTTTAGAGTTT | |
| | GGCAACATATGCCCATTCTTCCGCTTCCTCGCTCACTGACTCGCTGCGCTCGG | |
| | TCGTTCGGCTGCGGCGAGCGGTATCAGCTCACTCAAAGGCGGTAATACGGTTA | |
| | TCCACAGAATCAGGGGATAACGCAGGAAAGAACATGTGAGCAAAAGGCCAG | |
| | CAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCG | |
| | AAACCCGACAGGACTATAAAGATACCAGGCGTTTCCCCCTGGAAGCTCCCTC | |
| | GTGCGCTCTCCTGTTCCGACCCTGCCGCTTACCGGATACCTGTCCGCCTTTCTC | |
| | CCTTCGGGAAGCGTGGCGCTTTCTCATAGCTCACGCTGTAGGTATCTCAGTTC | |
| | GGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCG | |
| | AGGTATGTAGGCGGTGCTACAGAGTTCTTGAAGTGGTGGCCTAACTACGGCTA | |
| | CACTAGAAGAACAGTATTTGGTATCTGCGCTCTGCTGAAGCCAGTTACCTTCG | |
| | GAAAAAGAGTTGGTAGCTCTTGATCCGGCAAACAAACCACCGCTGGTAGCGG | |
| | TGGTTTTTTTGTTTGCAAGCAGCAGATTACGCGCAGAAAAAAAGGATCTCAA | |
| | GAAGATCCTTTGATCTTTTCTACGGGGTCTGACGCTCAGTGGAACGAAAACTC | |
| | ACGTTAAGGGATTTTGGTCATGAGATTATCAAAAAGGATCTTCACCTAGATCCT | |
| | TTTAAATTAAAAATGAAGTTTTAAATCAATCTAAAGTATATATGAGTAAACTTG | |
| | GTCTGACAGTTACCAATGCTTAATCAGTGAGGCACCTATCTCAGCGATCTGTC | |
| | TATTTCGTTCATCCATAGTTGCCTGACTCGGGGGGGGGGGGCGCTGAGGTCTG | |
| | CCTCGTGAAGAAGGTGTTGCTGACTCATACCAGGCCTGAATCGCCCCATCATC | |
| | CAGCCAGAAAGTGAGGGAGCCACGGTTGATGAGAGCTTTGTTGTAGGTGGAC | |
| | CAGTTGGTGATTTTGAACTTTTGCTTTGCCACGGAACGGTCTGCGTTGTCGGG | |
| | AAGATGCGTGATCTGATCCTTCAACTCAGCAAAAGTTCGATTTATTCAACAAA | |
| | GCCGCCGTCCCGTCAAGTCAGCGTAATGCTCTGCCAGTGTTACAACCAATTAA | |
| | CCAATTCTGATTAGAAAAACTCATCGAGCATCAAATGAAACTGCAATTTATTC | |
| | ATATCAGGATTATCAATACCATATTTTTGAAAAAGCCGTTTCTGTAATGAAGGA | |
| | GAAAACTCACCGAGGCAGTTCCATAGGATGGCAAGATCCTGGTATCGGTCTG | |
| | CGATTCCGACTCGTCCAACATCAATACAACCTATTAATTTCCCCTCGTCAAAAA | |
| | TAAGGTTATCAAGTGAGAAATCACCATGAGTGACGACTGAATCCGGTGAGAA | |
| | TGGCAAAAGCTTATGCATTTCTTTCCAGACTTGTTCAACAGGCCAGCCATTAC | |
| | GCTCGTCATCAAAATCACTCGCATCAACCAAACCGTTATTCATTCGTGATTGCG | |
| | CCTGAGCGAGACGAAATACGCGATCGCTGTTAAAAGGACAATTACAAACAGG | |
| | AATCGAATGCAACCGGCGCAGGAACACTGCCAGCGCATCAACAATATTTTCA | |
| | CCTGAATCAGGATATTCTTCTAATACCTGGAATGCTGTTTTCCCGGGGATCGCA | |
| | GTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACAT | |
| | CATTGGCAACGCTACCTTTGCCATGTTTCAGAAACAACTCTGGCGCATCGGGC | |
| | TTCCCATACAATCGATAGATTGTCGCACCTGATTGCCCGACATTATCGCGAGCC | |
| | CATTTATACCCATATAAATCAGCATCCATGTTGGAATTTAATCGCGGCCTCGAG | |
| | CAAGACGTTTCCCGTTGAATATGGCTCATAACACCCCTTGTATTACTGTTTATG | |
| | TAAGCAGACAGTTTTATTGTTCATGATGATATATTTTTATCTTGTGCAATGTAAC | |
| | ATCAGAGATTTTGAGACACAACGTGGCTTTCCCCCCCCCCCCATTATTGAAGC | |
| | ATTTATCAGGGTTATTGTCTCATGAGCGGATACATATTTGAATGTATTTAGAAAA | |
| | ATAAACAAATAGGGGTTCCGCGCACATTTCCCCGAAAAGTGCCACCTGACGT | |
| | CTAAGAAACCATTATTATCATGACATTAACCTATAAAAATAGGCGTATCACGAG | |
| | GCCCTTTCGTCTCGCGCGTTTCGGTGATGACGGTGAAAACCTCTGACACATGC | |
| | AGCTCCCGGAGACGGTCACAGCTTGTCTGTAAGCGGATGCCGGGAGCAGACA | |
| | AGCCCGTCAGGGCGCGTCAGCGGGTGTTGGCGGGTGTCGGGGCTGGCTTAAC | |
| | TATGCGGCATCAGAGCAGATTGTACTGAGAGTGCACCATATGCGGTGTGAAAT | |
| | ACCGCACAGATGCGTAAGGAGAAAATACCGCATCAGATTGGCTAT | |
| 15 | TGGCCATTGCATACGTTGTATCCATATCATAATATGTACATTTATATTGGCTCATG | pGBI-2007 (bi- |
| | TCCAACATTACCGCCATGTTGACATTGATTATTGACTAGTTATTAATAGTAATCA | shRNA construct #5 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTTTCTTCCCCATTAGGGATTATGCTGAATTTGTATGGTTT | |
| | ATAGTTGTTAGAGTTTGAGGTGTTAATTCTAATTATCTATTTCAAATTTAG | |
| | CAGGAAAAAAGAGAACATCACCTTGTAAAACTGAAGATTGTGACCAGTC | |
| | AGAATAATGTCTGCCGTGGAATGGTTTGATGCTAGATATGTGCATCTAGC | |
| | ATCAAACCATTCCACGGCAGCATTATGGTGACAGCTGCCTCGGGAAGCC | |
| | AAGTTGGGCTTTAAAGTGCAGGGCCTGCTGATGTTGAGTGCTTTTTGTT | |
| | CCTGATGTGGATCAGTTTGACACTAGTGAAGTAGATTAGCATCTAGCATC | |
| | AAACCATTCCACGGCAGCATAAGAAGTTATGTATTCATCCAATAATTCAAG | |
| | CCAAGCAAGTATATAGGTGTTTTAATAGTTTTTGTTTTGGGGATTGTGAC | |
| | CAGAAGATTTTGAAAATTAAATATTACTGAAGATTTCGACTTCCACTGTTA | |
| | AATGTACAAGATACATGAAATATTAAAGAAAATGTGTAACTTTTTGTGTAA | |
| | ATACATCTTGTGCGGCCGCGGATCCAGATCTTTTTCCCTCTGCCAAAAATTAT | |
| | GGGGACATCATGAAGCCCCTTGAGCATCTGACTTCTGGCTAATAAAGGAAATT | |
| | TATTTTCATTGCAATAGTGTGTTGGAATTTTTTGTGTCTCTCACTCGGAAGGAC | |
| | ATATGGGAGGGCAAATCATTTAAAACATCAGAATGAGTATTTGGTTTAGAGTTT | |
| | GGCAACATATGCCCATTCTTCCGCTTCCTCGCTCACTGACTCGCTGCGCTCGG | |
| | TCGTTCGGCTGCGGCGAGCGGTATCAGCTCACTCAAAGGCGGTAATACGGTTA | |
| | TCCACAGAATCAGGGGATAACGCAGGAAAGAACATGTGAGCAAAAGGCCAG | |
| | CAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCG | |
| | AAACCCGACAGGACTATAAAGATACCAGGCGTTTCCCCCTGGAAGCTCCCTC | |
| | GTGCGCTCTCCTGTTCCGACCCTGCCGCTTACCGGATACCTGTCCGCCTTTCTC | |
| | CCTTCGGGAAGCGTGGCGCTTTCTCATAGCTCACGCTGTAGGTATCTCAGTTC | |
| | GGTGTAGGTCGTTCGCTCCAAGCTGGGCTGTGTGCACGAACCCCCCGTTCAG | |
| | CCCGACCGCTGCGCCTTATCCGGTAACTATCGTCTTGAGTCCAACCCGGTAAG | |
| | ACACGACTTATCGCCACTGGCAGCAGCCACTGGTAACAGGATTAGCAGAGCG | |
| | AGGTATGTAGGCGGTGCTACAGAGTTCTTGAAGTGGTGGCCTAACTACGGCTA | |
| | CACTAGAAGAACAGTATTTGGTATCTGCGCTCTGCTGAAGCCAGTTACCTTCG | |
| | GAAAAAGAGTTGGTAGCTCTTGATCCGGCAAACAAACCACCGCTGGTAGCGG | |
| | TGGTTTTTTTGTTTGCAAGCAGCAGATTACGCGCAGAAAAAAAGGATCTCAA | |
| | GAAGATCCTTTGATCTTTTCTACGGGGTCTGACGCTCAGTGGAACGAAAACTC | |
| | ACGTTAAGGGATTTTGGTCATGAGATTATCAAAAAGGATCTTCACCTAGATCCT | |
| | TTTAAATTAAAAATGAAGTTTTAAATCAATCTAAAGTATATATGAGTAAACTTG | |
| | GTCTGACAGTTACCAATGCTTAATCAGTGAGGCACCTATCTCAGCGATCTGTC | |
| | TATTTCGTTCATCCATAGTTGCCTGACTCGGGGGGGGGGGGCGCTGAGGTCTG | |
| | CCTCGTGAAGAAGGTGTTGCTGACTCATACCAGGCCTGAATCGCCCCATCATC | |
| | CAGCCAGAAAGTGAGGGAGCCACGGTTGATGAGAGCTTTGTTGTAGGTGGAC | |
| | CAGTTGGTGATTTTGAACTTTTGCTTTGCCACGGAACGGTCTGCGTTGTCGGG | |
| | AAGATGCGTGATCTGATCCTTCAACTCAGCAAAAGTTCGATTTATTCAACAAA | |
| | GCCGCCGTCCCGTCAAGTCAGCGTAATGCTCTGCCAGTGTTACAACCAATTAA | |
| | CCAATTCTGATTAGAAAAACTCATCGAGCATCAAATGAAACTGCAATTTATTC | |
| | ATATCAGGATTATCAATACCATATTTTTGAAAAAGCCGTTTCTGTAATGAAGGA | |
| | GAAAACTCACCGAGGCAGTTCCATAGGATGGCAAGATCCTGGTATCGGTCTG | |
| | CGATTCCGACTCGTCCAACATCAATACAACCTATTAATTTCCCCTCGTCAAAAA | |
| | TAAGGTTATCAAGTGAGAAATCACCATGAGTGACGACTGAATCCGGTGAGAA | |
| | TGGCAAAAGCTTATGCATTTCTTTCCAGACTTGTTCAACAGGCCAGCCATTAC | |
| | GCTCGTCATCAAAATCACTCGCATCAACCAAACCGTTATTCATTCGTGATTGCG | |
| | CCTGAGCGAGACGAAATACGCGATCGCTGTTAAAAGGACAATTACAAACAGG | |
| | AATCGAATGCAACCGGCGCAGGAACACTGCCAGCGCATCAACAATATTTTCA | |
| | CCTGAATCAGGATATTCTTCTAATACCTGGAATGCTGTTTTCCCGGGGATCGCA | |
| | GTGGTGAGTAACCATGCATCATCAGGAGTACGGATAAAATGCTTGATGGTCGG | |
| | AAGAGGCATAAATTCCGTCAGCCAGTTTAGTCTGACCATCTCATCTGTAACAT | |
| | CATTGGCAACGCTACCTTTGCCATGTTTCAGAAACAACTCTGGCGCATCGGGC | |
| | TTCCCATACAATCGATAGATTGTCGCACCTGATTGCCCGACATTATCGCGAGCC | |
| | CATTTATACCCATATAAATCAGCATCCATGTTGGAATTTAATCGCGGCCTCGAG | |
| | CAAGACGTTTCCCGTTGAATATGGCTCATAACACCCCTTGTATTACTGTTTATG | |
| | TAAGCAGACAGTTTTATTGTTCATGATGATATATTTTTATCTTGTGCAATGTAAC | |
| | ATCAGAGATTTTGAGACACAACGTGGCTTTCCCCCCCCCCCCATTATTGAAGC | |
| | ATTTATCAGGGTTATTGTCTCATGAGCGGATACATATTTGAATGTATTTAGAAAA | |
| | ATAAACAAATAGGGGTTCCGCGCACATTTCCCCGAAAAGTGCCACCTGACGT | |
| | CTAAGAAACCATTATTATCATGACATTAACCTATAAAAATAGGCGTATCACGAG | |
| | GCCCTTTCGTCTCGCGCGTTTCGGTGATGACGGTGAAAACCTCTGACACATGC | |
| | AGCTCCCGGAGACGGTCACAGCTTGTCTGTAAGCGGATGCCGGGAGCAGACA | |
| | AGCCCGTCAGGGCGCGTCAGCGGGTGTTGGCGGGTGTCGGGGCTGGCTTAAC | |
| | TATGCGGCATCAGAGCAGATTGTACTGAGAGTGCACCATATGCGGTGTGAAAT | |
| | ACCGCACAGATGCGTAAGGAGAAAATACCGCATCAGATTGGCTAT | |
| 16 | TGGCCATTGCATACGTTGTATCCATATCATAATATGTACATTTATATTGGCTCATG | pGBI-2008 (bi- |
| | TCCAACATTACCGCCATGTTGACATTGATTATTGACTAGTTATTAATAGTAATCA | shRNA construct #6 |
| | ATTACGGGGTCATTAGTTCATAGCCCATATATGGAGTTCCGCGTTACATAACTTA | targeting the |
| | CGGTAAATGGCCCGCCTGGCTGACCGCCCAACGACCCCCGCCCATTGACGTC | EWSR1-ATF1 rare |
| | AATAATGACGTATGTTCCCATAGTAACGCCAATAGGGACTTTCCATTGACGTCA | fusion gene; bi- |
| | ATGGGTGGAGTATTTACGGTAAACTGCCCACTTGGCAGTACATCAAGTGTATC | shRNA sequence in |
| | ATATGCCAAGTACGCCCCCTATTGACGTCAATGACGGTAAATGGCCCGCCTGG | bold) |
| | CATTATGCCCAGTACATGACCTTATGGGACTTTCCTACTTGGCAGTACATCTAC | |
| | GTATTAGTCATCGCTATTACCATGGTGATGCGGTTTTGGCAGTACATCAATGGG | |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACTCACCG | |
| | TCGTCGACTTTCTTCCCCATTAGGGATTATGCTGAATTTGTATGGTTTATA | |
| | GTTGTTAGAGTTTGAGGTGTTAATTCTAATTATCTATTTCAAATTTAGCAG | |
| | GAAAAAAGAGAACATCACCTTGTAAAACTGAAGATTGTGACCAGTCAGA | |
| | ATAATGTTGCCGTGGAATGGTTTGATGCTGAGATATGTGCATCTCAGCAT | |
| | CAAACCATTCCACGGCACATTATGGTGACAGCTGCCTCGGGAAGCCAAG | |
| | TTGGGCTTTAAAGTGCAGGGCCTGCTGATGTTGAGTGCTTTTTGTTCTG | |
| | ATGTGTAACAATTTGACGATGAGTGAAGTAGATTAGCATCTCAGCATCAA | |
| | ACCATTCCACGGCACATAAGAAGTTATGTATTCATCCAATAATTCAAGCCA | |
| | AGCAAGTATATAGGTGTTTTAATAGTTTTTGTTTTGGGGATTGTGACCAG | |
| | AAGATTTTGAAAATTAAATATTACTGAAGATTTCGACTTCCACTGTTAAAT | |
| | GTACAAGATACATGAAATATTAAAGAAAATGTGTAACTTTTTGTGTAAATA | |
| | CATCTTGTGCGGCCGCGGATCCAGATCTTTTTCCCTCTGCCAAAAATTATGGG | |
| | GACATCATGAAGCCCCTTGAGCATCTGACTTCTGGCTAATAAAGGAAATTTATT | |
| | TTCATTGCAATAGTGTGTTGGAATTTTTTGTGTCTCTCACTCGGAAGGACATAT | |
| | GGGAGGGCAAATCATTTAAAACATCAGAATGAGTATTTGGTTTAGAGTTTGGC | |
| | AACATATGCCCATTCTTCCGCTTCCTCGCTCACTGACTCGCTGCGCTCGGTCGT | |
| | TCGGCTGCGGCGAGCGGTATCAGCTCACTCAAAGGCGGTAATACGGTTATCCA | |
| | CAGAATCAGGGGATAACGCAGGAAAGAACATGTGAGCAAAAGGCCAGCAAA | |
| | AGGCCAGGAACCGTAAAAAGGCCGCGTTGCTGGCGTTTTTCCATAGGCTCCG | |
| | CCCCCCTGACGAGCATCACAAAAATCGACGCTCAAGTCAGAGGTGGCGAAA | |
| | CCCGACAGGACTATAAAGATACCAGGCGTTTCCCCCTGGAAGCTCCCTCGTGC | |
| | GCTCTCCTGTTCCGACCCTGCCGCTTACCGGATACCTGTCCGCCTTTCTCCCTT | |
| | CGGGAAGCGTGGCGCTTTCTCATAGCTCACGCTGTAGGTATCTCAGTTCGGTG | |
| | TAGGTCGTTCGCTCCAAGCTGGGCTGTGTGCACGAACCCCCCGTTCAGCCCG | |
| | ACCGCTGCGCCTTATCCGGTAACTATCGTCTTGAGTCCAACCCGGTAAGACAC | |
| | GACTTATCGCCACTGGCAGCAGCCACTGGTAACAGGATTAGCAGAGCGAGGT | |
| | ATGTAGGCGGTGCTACAGAGTTCTTGAAGTGGTGGCCTAACTACGGCTACACT | |
| | AGAAGAACAGTATTTGGTATCTGCGCTCTGCTGAAGCCAGTTACCTTCGGAAA | |
| | AAGAGTTGGTAGCTCTTGATCCGGCAAACAAACCACCGCTGGTAGCGGTGGT | |
| | TTTTTTGTTTGCAAGCAGCAGATTACGCGCAGAAAAAAAGGATCTCAAGAAG | |
| | ATCCTTTGATCTTTTCTACGGGGTCTGACGCTCAGTGGAACGAAAACTCACGT | |
| | TAAGGGATTTTGGTCATGAGATTATCAAAAAGGATCTTCACCTAGATCCTTTTA | |
| | AATTAAAAATGAAGTTTTAAATCAATCTAAAGTATATATGAGTAAACTTGGTCT | |
| | GACAGTTACCAATGCTTAATCAGTGAGGCACCTATCTCAGCGATCTGTCTATTT | |
| | CGTTCATCCATAGTTGCCTGACTCGGGGGGGGGGGGCGCTGAGGTCTGCCTC | |
| | GTGAAGAAGGTGTTGCTGACTCATACCAGGCCTGAATCGCCCCATCATCCAGC | |
| | CAGAAAGTGAGGGAGCCACGGTTGATGAGAGCTTTGTTGTAGGTGGACCAGT | |
| | TGGTGATTTTGAACTTTTGCTTTGCCACGGAACGGTCTGCGTTGTCGGGAAGA | |
| | TGCGTGATCTGATCCTTCAACTCAGCAAAAGTTCGATTTATTCAACAAAGCCG | |
| | CCGTCCCGTCAAGTCAGCGTAATGCTCTGCCAGTGTTACAACCAATTAACCAA | |
| | TTCTGATTAGAAAAACTCATCGAGCATCAAATGAAACTGCAATTTATTCATATC | |
| | AGGATTATCAATACCATATTTTTGAAAAAGCCGTTTCTGTAATGAAGGAGAAA | |
| | ACTCACCGAGGCAGTTCCATAGGATGGCAAGATCCTGGTATCGGTCTGCGATT | |
| | CCGACTCGTCCAACATCAATACAACCTATTAATTTCCCCTCGTCAAAAATAAG | |
| | GTTATCAAGTGAGAAATCACCATGAGTGACGACTGAATCCGGTGAGAATGGC | |
| | AAAAGCTTATGCATTTCTTTCCAGACTTGTTCAACAGGCCAGCCATTACGCTC | |
| | GTCATCAAAATCACTCGCATCAACCAAACCGTTATTCATTCGTGATTGCGCCTG | |
| | AGCGAGACGAAATACGCGATCGCTGTTAAAAGGACAATTACAAACAGGAATC | |
| | GAATGCAACCGGCGCAGGAACACTGCCAGCGCATCAACAATATTTTCACCTG | |
| | AATCAGGATATTCTTCTAATACCTGGAATGCTGTTTTCCCGGGGATCGCAGTGG | |
| | TGAGTAACCATGCATCATCAGGAGTACGGATAAAATGCTTGATGGTCGGAAGA | |
| | GGCATAAATTCCGTCAGCCAGTTTAGTCTGACCATCTCATCTGTAACATCATTG | |
| | GCAACGCTACCTTTGCCATGTTTCAGAAACAACTCTGGCGCATCGGGCTTCCC | |
| | ATACAATCGATAGATTGTCGCACCTGATTGCCCGACATTATCGCGAGCCCATTT | |
| | ATACCCATATAAATCAGCATCCATGTTGGAATTTAATCGCGGCCTCGAGCAAGA | |
| | CGTTTCCCGTTGAATATGGCTCATAACACCCCTTGTATTACTGTTTATGTAAGC | |
| | AGACAGTTTTATTGTTCATGATGATATATTTTTATCTTGTGCAATGTAACATCAG | |
| | AGATTTTGAGACACAACGTGGCTTTCCCCCCCCCCCCATTATTGAAGCATTTAT | |
| | CAGGGTTATTGTCTCATGAGCGGATACATATTTGAATGTATTTAGAAAAATAAA | |
| | CAAATAGGGGTTCCGCGCACATTTCCCCGAAAAGTGCCACCTGACGTCTAAG | |
| | AAACCATTATTATCATGACATTAACCTATAAAAATAGGCGTATCACGAGGCCCT | |
| | TTCGTCTCGCGCGTTTCGGTGATGACGGTGAAAACCTCTGACACATGCAGCTC | |
| | CCGGAGACGGTCACAGCTTGTCTGTAAGCGGATGCCGGGAGCAGACAAGCC | |
| | CGTCAGGGCGCGTCAGCGGGTGTTGGCGGGTGTCGGGGCTGGCTTAACTATG | |
| | CGGCATCAGAGCAGATTGTACTGAGAGTGCACCATATGCGGTGTGAAATACCG | |
| | CACAGATGCGTAAGGAGAAAATACCGCATCAGATTGGCTAT | |
| 17 | TGGCCATTGCATACGTTGTATCCATATCATAATATGTACATTTATATTGGCTCATG | pGBI-2011 (triple |
| | TCCAACATTACCGCCATGTTGACATTGATTATTGACTAGTTATTAATAGTAATCA | knockdown bi- |
| | ATTACGGGGTCATTAGTTCATAGCCCATATATGGAGTTCCGCGTTACATAACTTA | shRNA construct #1 |
| | AATAATGACGTATGTTCCCATAGTAACGCCAATAGGGACTTTCCATTGACGTCA | targeting Type 1, |
| | CGGTAAATGGCCCGCCTGGCTGACCGCCCAACGACCCCCGCCCATTGACGTC | Type 2, and the rare |
| | ATGGGTGGAGTATTTACGGTAAACTGCCCACTTGGCAGTACATCAAGTGTATC | EWSR1-ATF1 |
| | ATATGCCAAGTACGCCCCCTATTGACGTCAATGACGGTAAATGGCCCGCCTGG | fusion genes; bi- |
| | CATTATGCCCAGTACATGACCTTATGGGACTTTCCTACTTGGCAGTACATCTAC | shRNA sequence in |
| | GTATTAGTCATCGCTATTACCATGGTGATGCGGTTTTGGCAGTACATCAATGGG | bold) |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTCTTCCCCATTAGGGATTATGCTGAATTTGTATGGTTTATA | |
| | GTTGTTAGAGTTTGAGGTGTTAATTCTAATTATCTATTTCAAATTTAGCAG | |
| | GAAAAAAGAGAACATCACCTTGTAAAACTGAAGATTGTGACCAGTCAGA | |
| | ATAATGTCCGTGGAATGGTTTGATGCTGCATGATATGTGCATCTTGCAGC | |
| | ATCAAACCATTCCACGGCATTATGGTGACAGCTGCCTCGGGAAGCCAAG | |
| | TTGGGCTTTAAAGTGCAGGGCCTGCTGATGTTGAGTGCTTTTTGTTCCC | |
| | ATGTAATGACCTGATGATACATGAAGTAGATTAGCATCTTGCAGCATCAA | |
| | ACCATTCCACGGCATAAGAAGTTATGTATTCATCCAATAATTCAAGCCAAG | |
| | CAAGTATATAGGTGTTTTAATAGTTTTTGTTTGCAGTCCTCTGTTAGGACG | |
| | CGGTGGAATGGGAAAAAAGAAGAATGTAGTTTTTTCCCATTCCACCGCG | |
| | TCCTTGGTGGCCTGCTATTTCCTTCAAATGAATGATTTTTACTAATTTTGT | |
| | GTACTTTTATTGTGTCGATGTAGAATCTGCCTGGTCTATCTGATGTGACA | |
| | GCTTCTGTAGCACAGGACGATGAACAATGGGTGAAATGTTTAGTTATCTT | |
| | TTTTCCCATTCCACCGCGTCCTTACTGCTAGCTGTAGAACTCCAGCTTCG | |
| | GCCTGTCGCCCAATCAAACTGTCCTGTTACTGAACACTGTTCTATGGTTG | |
| | CAGCTACGGGCAGCAGATTGCCTGTGTGATATTCTGCGGCAATCTGCTG | |
| | CCCGTAGCTGCCTGTGGTAGTGAAAAGTCTGTAGAAAAGTAAGGGAAAC | |
| | TCAAACCCCTTTCTACACGCAGACACGAGTAGCAGATCACCGTGTTTCT | |
| | GTATGGGGCAATCTGCTGCCCGTAGCTGCTTGAGTTTGGTGGGGATTGT | |
| | GACCAGAAGATTTTGAAAATTAAATATTACTGAAGATTTCGACTTCCACT | |
| | GTTAAATGTACAAGATACATGAAATATTAAAGAAAATGTGTAACTTTTTGT | |
| | GTAAATACATCTTGTCTTGTTTTCATTCAAAAACATTTCACTTTTGGGGTT | |
| | GCGTGTCAGATTTGGCAGTATAAATTCTGGCTATATTTTTTGCGGCCGCGG | |
| | ATCCAGATCTTTTTCCCTCTGCCAAAAATTATGGGGACATCATGAAGCCCCTTG | |
| | AGCATCTGACTTCTGGCTAATAAAGGAAATTTATTTTCATTGCAATAGTGTGTT | |
| | GGAATTTTTTGTGTCTCTCACTCGGAAGGACATATGGGAGGGCAAATCATTTA | |
| | AAACATCAGAATGAGTATTTGGTTTAGAGTTTGGCAACATATGCCCATTCTTCC | |
| | GCTTCCTCGCTCACTGACTCGCTGCGCTCGGTCGTTCGGCTGCGGCGAGCGG | |
| | TATCAGCTCACTCAAAGGCGGTAATACGGTTATCCACAGAATCAGGGGATAAC | |
| | GCAGGAAAGAACATGTGAGCAAAAGGCCAGCAAAAGGCCAGGAACCGTAA | |
| | AAAGGCCGCGTTGCTGGCGTTTTTCCATAGGCTCCGCCCCCCTGACGAGCATC | |
| | ACAAAAATCGACGCTCAAGTCAGAGGTGGCGAAACCCGACAGGACTATAAA | |
| | GATACCAGGCGTTTCCCCCTGGAAGCTCCCTCGTGCGCTCTCCTGTTCCGACC | |
| | CTGCCGCTTACCGGATACCTGTCCGCCTTTCTCCCTTCGGGAAGCGTGGCGCT | |
| | TTCTCATAGCTCACGCTGTAGGTATCTCAGTTCGGTGTAGGTCGTTCGCTCCAA | |
| | GCTGGGCTGTGTGCACGAACCCCCCGTTCAGCCCGACCGCTGCGCCTTATCC | |
| | GGTAACTATCGTCTTGAGTCCAACCCGGTAAGACACGACTTATCGCCACTGGC | |
| | AGCAGCCACTGGTAACAGGATTAGCAGAGCGAGGTATGTAGGCGGTGCTACA | |
| | GAGTTCTTGAAGTGGTGGCCTAACTACGGCTACACTAGAAGAACAGTATTTGG | |
| | TATCTGCGCTCTGCTGAAGCCAGTTACCTTCGGAAAAAGAGTTGGTAGCTCTT | |
| | GATCCGGCAAACAAACCACCGCTGGTAGCGGTGGTTTTTTTGTTTGCAAGCA | |
| | GCAGATTACGCGCAGAAAAAAAGGATCTCAAGAAGATCCTTTGATCTTTTCTA | |
| | CGGGGTCTGACGCTCAGTGGAACGAAAACTCACGTTAAGGGATTTTGGTCAT | |
| | GAGATTATCAAAAAGGATCTTCACCTAGATCCTTTTAAATTAAAAATGAAGTTT | |
| | TAAATCAATCTAAAGTATATATGAGTAAACTTGGTCTGACAGTTACCAATGCTT | |
| | AATCAGTGAGGCACCTATCTCAGCGATCTGTCTATTTCGTTCATCCATAGTTGC | |
| | CTGACTCGGGGGGGGGGGGCGCTGAGGTCTGCCTCGTGAAGAAGGTGTTGC | |
| | TGACTCATACCAGGCCTGAATCGCCCCATCATCCAGCCAGAAAGTGAGGGAG | |
| | CCACGGTTGATGAGAGCTTTGTTGTAGGTGGACCAGTTGGTGATTTTGAACTT | |
| | TTGCTTTGCCACGGAACGGTCTGCGTTGTCGGGAAGATGCGTGATCTGATCCT | |
| | TCAACTCAGCAAAAGTTCGATTTATTCAACAAAGCCGCCGTCCCGTCAAGTC | |
| | AGCGTAATGCTCTGCCAGTGTTACAACCAATTAACCAATTCTGATTAGAAAAA | |
| | CTCATCGAGCATCAAATGAAACTGCAATTTATTCATATCAGGATTATCAATACC | |
| | ATATTTTTGAAAAAGCCGTTTCTGTAATGAAGGAGAAAACTCACCGAGGCAG | |
| | TTCCATAGGATGGCAAGATCCTGGTATCGGTCTGCGATTCCGACTCGTCCAAC | |
| | ATCAATACAACCTATTAATTTCCCCTCGTCAAAAATAAGGTTATCAAGTGAGAA | |
| | ATCACCATGAGTGACGACTGAATCCGGTGAGAATGGCAAAAGCTTATGCATTT | |
| | CTTTCCAGACTTGTTCAACAGGCCAGCCATTACGCTCGTCATCAAAATCACTC | |
| | GCATCAACCAAACCGTTATTCATTCGTGATTGCGCCTGAGCGAGACGAAATAC | |
| | GCGATCGCTGTTAAAAGGACAATTACAAACAGGAATCGAATGCAACCGGCGC | |
| | AGGAACACTGCCAGCGCATCAACAATATTTTCACCTGAATCAGGATATTCTTC | |
| | TAATACCTGGAATGCTGTTTTCCCGGGGATCGCAGTGGTGAGTAACCATGCAT | |
| | CATCAGGAGTACGGATAAAATGCTTGATGGTCGGAAGAGGCATAAATTCCGTC | |
| | AGCCAGTTTAGTCTGACCATCTCATCTGTAACATCATTGGCAACGCTACCTTTG | |
| | CCATGTTTCAGAAACAACTCTGGCGCATCGGGCTTCCCATACAATCGATAGAT | |
| | TGTCGCACCTGATTGCCCGACATTATCGCGAGCCCATTTATACCCATATAAATC | |
| | AGCATCCATGTTGGAATTTAATCGCGGCCTCGAGCAAGACGTTTCCCGTTGAA | |
| | TATGGCTCATAACACCCCTTGTATTACTGTTTATGTAAGCAGACAGTTTTATTGT | |
| | TCATGATGATATATTTTTATCTTGTGCAATGTAACATCAGAGATTTTGAGACACA | |
| | ACGTGGCTTTCCCCCCCCCCCCATTATTGAAGCATTTATCAGGGTTATTGTCTC | |
| | ATGAGCGGATACATATTTGAATGTATTTAGAAAAATAAACAAATAGGGGTTCCG | |
| | CGCACATTTCCCCGAAAAGTGCCACCTGACGTCTAAGAAACCATTATTATCAT | |
| | GACATTAACCTATAAAAATAGGCGTATCACGAGGCCCTTTCGTCTCGCGCGTT | |
| | TCGGTGATGACGGTGAAAACCTCTGACACATGCAGCTCCCGGAGACGGTCAC | |
| | AGCTTGTCTGTAAGCGGATGCCGGGAGCAGACAAGCCCGTCAGGGCGCGTCA | |
| | GCGGGTGTTGGCGGGTGTCGGGGCTGGCTTAACTATGCGGCATCAGAGCAGA | |
| | TTGTACTGAGAGTGCACCATATGCGGTGTGAAATACCGCACAGATGCGTAAGG | |
| | AGAAAATACCGCATCAGATTGGCTAT | |
| 18 | TGGCCATTGCATACGTTGTATCCATATCATAATATGTACATTTATATTGGCTCATG | pGBI-2012 (triple |
| | TCCAACATTACCGCCATGTTGACATTGATTATTGACTAGTTATTAATAGTAATCA | knockdown bi- |
| | ATTACGGGGTCATTAGTTCATAGCCCATATATGGAGTTCCGCGTTACATAACTTA | shRNA construct #2 |
| | CGGTAAATGGCCCGCCTGGCTGACCGCCCAACGACCCCCGCCCATTGACGTC | targeting Type 1, |
| | AATAATGACGTATGTTCCCATAGTAACGCCAATAGGGACTTTCCATTGACGTCA | Type 2, and the rare |
| | ATGGGTGGAGTATTTACGGTAAACTGCCCACTTGGCAGTACATCAAGTGTATC | EWSR1-ATF1 |
| | ATATGCCAAGTACGCCCCCTATTGACGTCAATGACGGTAAATGGCCCGCCTGG | fusion genes; bi- |
| | CATTATGCCCAGTACATGACCTTATGGGACTTTCCTACTTGGCAGTACATCTAC | shRNA sequence in |
| | GTATTAGTCATCGCTATTACCATGGTGATGCGGTTTTGGCAGTACATCAATGGG | bold) |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACTCACCG | |
| | TCGTCGACTCTTCCCCATTAGGGATTATGCTGAATTTGTATGGTTTATAGT | |
| | TGTTAGAGTTTGAGGTGTTAATTCTAATTATCTATTTCAAATTTAGCAGGA | |
| | AAAAAGAGAACATCACCTTGTAAAACTGAAGATTGTGACCAGTCAGAATA | |
| | ATGTCCGTGGAATGGTTTGATGCTGCATGATATGTGCATCTTGCAGCATC | |
| | AAACCATTCCACGGCATTATGGTGACAGCTGCCTCGGGAAGCCAAGTTG | |
| | GGCTTTAAAGTGCAGGGCCTGCTGATGTTGAGTGCTTTTTGTTCCCATGT | |
| | AATGACCTGATGATACATGAAGTAGATTAGCATCTTGCAGCATCAAACCA | |
| | TTCCACGGCATAAGAAGTTATGTATTCATCCAATAATTCAAGCCAAGCAA | |
| | GTATATAGGTGTTTTAATAGTTTTTGTTTGCAGTCCTCTGTTAGGACGCG | |
| | GTGGAATGGGAAAAAAGAAGAATGTAGTTTTTTCCCATTCCACCGCGTC | |
| | CTTGGTGGCCTGCTATTTCCTTCAAATGAATGATTTTTACTAATTTTGTGT | |
| | ACTTTTATTGTGTCGATGTAGAATCTGCCTGGTCTATCTGATGTGACAGC | |
| | TTCTGTAGCACAGGACGATGAACAATGGGTGAAATGTTTAGTTATCTTTT | |
| | TTCCCATTCCACCGCGTCCTTACTGCTAGCTGTAGAACTCCAGCTTCGGC | |
| | CTGTCGCCCAATCAAACTGTCCTGTTACTGAACACTGTTCTATGGTTGCA | |
| | GCTACGGGCAGCAGATTGCCTGTGTGATATTCTGCGGCAATCTGCTGCC | |
| | CGTAGCTGCCTGTGGTAGTGAAAAGTCTGTAGAAAAGTAAGGGAAACTC | |
| | AAACCCCTTTCTACACGCAGACACGAGTAGCAGATCACCGTGTTTCTGTA | |
| | TGGGGCAATCTGCTGCCCGTAGCTGCTTGAGTTTGGTGGGGATTGTGAC | |
| | CAGAAGATTTTGAAAATTAAATATTACTGAAGATTTCGACTTCCACTGTTA | |
| | AATGTACAAGATACATGAAATATTAAAGAAAATGTGTAACTTTTTGTGTAA | |
| | ATACATCTTGTCTTGTTTTCATTCAAAAACATTTCACTTTTGGGGTTGCGT | |
| | GTCAGATTTGGCAGTATAAATTCTGGCTATATTTTTTGCGGCCGCGGATCC | |
| | AGATCTTTTTCCCTCTGCCAAAAATTATGGGGACATCATGAAGCCCCTTGAGC | |
| | ATCTGACTTCTGGCTAATAAAGGAAATTTATTTTCATTGCAATAGTGTGTTGGA | |
| | ATTTTTTGTGTCTCTCACTCGGAAGGACATATGGGAGGGCAAATCATTTAAAA | |
| | CATCAGAATGAGTATTTGGTTTAGAGTTTGGCAACATATGCCCATTCTTCCGCT | |
| | TCCTCGCTCACTGACTCGCTGCGCTCGGTCGTTCGGCTGCGGCGAGCGGTATC | |
| | AGCTCACTCAAAGGCGGTAATACGGTTATCCACAGAATCAGGGGATAACGCA | |
| | GGAAAGAACATGTGAGCAAAAGGCCAGCAAAAGGCCAGGAACCGTAAAAA | |
| | GGCCGCGTTGCTGGCGTTTTTCCATAGGCTCCGCCCCCCTGACGAGCATCACA | |
| | AAAATCGACGCTCAAGTCAGAGGTGGCGAAACCCGACAGGACTATAAAGATA | |
| | CCAGGCGTTTCCCCCTGGAAGCTCCCTCGTGCGCTCTCCTGTTCCGACCCTGC | |
| | CGCTTACCGGATACCTGTCCGCCTTTCTCCCTTCGGGAAGCGTGGCGCTTTCT | |
| | CATAGCTCACGCTGTAGGTATCTCAGTTCGGTGTAGGTCGTTCGCTCCAAGCT | |
| | GGGCTGTGTGCACGAACCCCCCGTTCAGCCCGACCGCTGCGCCTTATCCGGT | |
| | AACTATCGTCTTGAGTCCAACCCGGTAAGACACGACTTATCGCCACTGGCAGC | |
| | AGCCACTGGTAACAGGATTAGCAGAGCGAGGTATGTAGGCGGTGCTACAGAG | |
| | TTCTTGAAGTGGTGGCCTAACTACGGCTACACTAGAAGAACAGTATTTGGTAT | |
| | CTGCGCTCTGCTGAAGCCAGTTACCTTCGGAAAAAGAGTTGGTAGCTCTTGAT | |
| | CCGGCAAACAAACCACCGCTGGTAGCGGTGGTTTTTTTGTTTGCAAGCAGCA | |
| | GATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGAG | |
| | ATTATCAAAAAGGATCTTCACCTAGATCCTTTTAAATTAAAAATGAAGTTTTAA | |
| | ATCAATCTAAAGTATATATGAGTAAACTTGGTCTGACAGTTACCAATGCTTAAT | |
| | CAGTGAGGCACCTATCTCAGCGATCTGTCTATTTCGTTCATCCATAGTTGCCTG | |
| | ACTCGGGGGGGGGGGGCGCTGAGGTCTGCCTCGTGAAGAAGGTGTTGCTGA | |
| | CTCATACCAGGCCTGAATCGCCCCATCATCCAGCCAGAAAGTGAGGGAGCCA | |
| | CGGTTGATGAGAGCTTTGTTGTAGGTGGACCAGTTGGTGATTTTGAACTTTTG | |
| | CTTTGCCACGGAACGGTCTGCGTTGTCGGGAAGATGCGTGATCTGATCCTTCA | |
| | ACTCAGCAAAAGTTCGATTTATTCAACAAAGCCGCCGTCCCGTCAAGTCAGC | |
| | GTAATGCTCTGCCAGTGTTACAACCAATTAACCAATTCTGATTAGAAAAACTC | |
| | ATCGAGCATCAAATGAAACTGCAATTTATTCATATCAGGATTATCAATACCATAT | |
| | TTTTGAAAAAGCCGTTTCTGTAATGAAGGAGAAAACTCACCGAGGCAGTTCC | |
| | ATAGGATGGCAAGATCCTGGTATCGGTCTGCGATTCCGACTCGTCCAACATCA | |
| | ATACAACCTATTAATTTCCCCTCGTCAAAAATAAGGTTATCAAGTGAGAAATCA | |
| | CCATGAGTGACGACTGAATCCGGTGAGAATGGCAAAAGCTTATGCATTTCTTT | |
| | CCAGACTTGTTCAACAGGCCAGCCATTACGCTCGTCATCAAAATCACTCGCAT | |
| | CAACCAAACCGTTATTCATTCGTGATTGCGCCTGAGCGAGACGAAATACGCGA | |
| | TCGCTGTTAAAAGGACAATTACAAACAGGAATCGAATGCAACCGGCGCAGGA | |
| | ACACTGCCAGCGCATCAACAATATTTTCACCTGAATCAGGATATTCTTCTAATA | |
| | CCTGGAATGCTGTTTTCCCGGGGATCGCAGTGGTGAGTAACCATGCATCATCA | |
| | GGAGTACGGATAAAATGCTTGATGGTCGGAAGAGGCATAAATTCCGTCAGCC | |
| | AGTTTAGTCTGACCATCTCATCTGTAACATCATTGGCAACGCTACCTTTGCCAT | |
| | GTTTCAGAAACAACTCTGGCGCATCGGGCTTCCCATACAATCGATAGATTGTC | |
| | GCACCTGATTGCCCGACATTATCGCGAGCCCATTTATACCCATATAAATCAGCA | |
| | TCCATGTTGGAATTTAATCGCGGCCTCGAGCAAGACGTTTCCCGTTGAATATG | |
| | GCTCATAACACCCCTTGTATTACTGTTTATGTAAGCAGACAGTTTTATTGTTCAT | |
| | GATGATATATTTTTATCTTGTGCAATGTAACATCAGAGATTTTGAGACACAACG | |
| | TGGCTTTCCCCCCCCCCCCATTATTGAAGCATTTATCAGGGTTATTGTCTCATG | |
| | AGCGGATACATATTTGAATGTATTTAGAAAAATAAACAAATAGGGGTTCCGCG | |
| | CACATTTCCCCGAAAAGTGCCACCTGACGTCTAAGAAACCATTATTATCATGA | |
| | CATTAACCTATAAAAATAGGCGTATCACGAGGCCCTTTCGTCTCGCGCGTTTCG | |
| | GTGATGACGGTGAAAACCTCTGACACATGCAGCTCCCGGAGACGGTCACAGC | |
| | TTGTCTGTAAGCGGATGCCGGGAGCAGACAAGCCCGTCAGGGCGCGTCAGCG | |
| | GGTGTTGGCGGGTGTCGGGGCTGGCTTAACTATGCGGCATCAGAGCAGATTG | |
| | TACTGAGAGTGCACCATATGCGGTGTGAAATACCGCACAGATGCGTAAGGAG | |
| | AAAATACCGCATCAGATTGGCTAT | |
| 19 | TGGCCATTGCATACGTTGTATCCATATCATAATATGTACATTTATATTGGCTCATG | pGBI-2013 (triple |
| | TCCAACATTACCGCCATGTTGACATTGATTATTGACTAGTTATTAATAGTAATCA | knockdown bi- |
| | ATTACGGGGTCATTAGTTCATAGCCCATATATGGAGTTCCGCGTTACATAACTTA | shRNA construct #3 |
| | CGGTAAATGGCCCGCCTGGCTGACCGCCCAACGACCCCCGCCCATTGACGTC | targeting Type 1, |
| | AATAATGACGTATGTTCCCATAGTAACGCCAATAGGGACTTTCCATTGACGTCA | Type 2, and the rare |
| | ATGGGTGGAGTATTTACGGTAAACTGCCCACTTGGCAGTACATCAAGTGTATC | EWSR1-ATF1 |
| | ATATGCCAAGTACGCCCCCTATTGACGTCAATGACGGTAAATGGCCCGCCTGG | fusion genes; bi- |
| | CATTATGCCCAGTACATGACCTTATGGGACTTTCCTACTTGGCAGTACATCTAC | shRNA sequence in |
| | GTATTAGTCATCGCTATTACCATGGTGATGCGGTTTTGGCAGTACATCAATGGG | bold) |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACGTAAGT | |
| | ACCGCCTATAGACTCTATAGGCACACCCCTTTGGCTCTTATGCATGCTATACTG | |
| | TTTTTGGCTTGGGGCCTATACACCCCCGCTTCCTTATGCTATAGGTGATGGTATA | |
| | GCTTAGCCTATAGGTGTGGGTTATTGACCATTATTGACCACTCCAACGGTGGA | |
| | GGGCAGTGTAGTCTGAGCAGTACTCGTTGCTGCCGCGCGCGCCACCAGACAT | |
| | AATAGCTGACAGACTAACAGACTGTTCCTTTCCATGGGTCTTTTCTGCAGTCA | |
| | CCGTCGTCGACTCTTCCCCATTAGGGATTATGCTGAATTTGTATGGTTTATA | |
| | GTTGTTAGAGTTTGAGGTGTTAATTCTAATTATCTATTTCAAATTTAGCAG | |
| | GAAAAAAGAGAACATCACCTTGTAAAACTGAAGATTGTGACCAGTCAGA | |
| | ATAATGTCCGTGGAATGGTTTGATGCTGCATGATATGTGCATCTTGCAGC | |
| | ATCAAACCATTCCACGGCATTATGGTGACAGCTGCCTCGGGAAGCCAAG | |
| | TTGGGCTTTAAAGTGCAGGGCCTGCTGATGTTGAGTGCTTTTTGTTCCC | |
| | ATGTAATGACCTGATGATACATGAAGTAGATTAGCATCTTGCAGCATCAA | |
| | ACCATTCCACGGCATAAGAAGTTATGTATTCATCCAATAATTCAAGCCAAG | |
| | CAAGTATATAGGTGTTTTAATAGTTTTTGTTTGCAGTCCTCTGTTGAGGAC | |
| | GCGGTGGAATGGGAAAAAGAAGAATGTAGTTTTTCCCATTCCACCGCGT | |
| | CCTCTGGTGGCCTGCTATTTCCTTCAAATGAATGATTTTTACTAATTTTGT | |
| | GTACTTTTATTGTGTCGATGTAGAATCTGCCTGGTCTATCTGATGTGACA | |
| | GCTTCTGTAGCACGAGTTAGCGAGTGAATGGTCAAATGTTTAGTTATCTT | |
| | TTTCCCATTCCACCGCGTCCTCTACTGCTAGCTGTAGAACTCCAGCTTCG | |
| | GCCTGTCGCCCAATCAAACTGTCCTGTTACTGAACACTGTTCTATGGTTA | |
| | GCAGCTACGGGCAGCAGATTGCTGTGTGATATTCTGCGCAATCTGCTGC | |
| | CCGTAGCTGCTCTGTGGTAGTGAAAAGTCTGTAGAAAAGTAAGGGAAAC | |
| | TCAAACCCCTTTCTACACAGCATACACATACAGCATCATGCGTGTTTCTG | |
| | TATGGGCAATCTGCTGCCCGTAGCTGCTTTGAGTTTGGTGGGGATTGTG | |
| | ACCAGAAGATTTTGAAAATTAAATATTACTGAAGATTTCGACTTCCACTGT | |
| | TAAATGTACAAGATACATGAAATATTAAAGAAAATGTGTAACTTTTTGTGT | |
| | AAATACATCTTGTCTTGTTTTCATTCAAAAACATTTCACTTTTGGGGTTGC | |
| | GTGTCAGATTTGGCAGTATAAATTCTGGCTATATTTTTTGCGGCCGCGGAT | |
| | CCAGATCTTTTTCCCTCTGCCAAAAATTATGGGGACATCATGAAGCCCCTTGA | |
| | GCATCTGACTTCTGGCTAATAAAGGAAATTTATTTTCATTGCAATAGTGTGTTG | |
| | GAATTTTTTGTGTCTCTCACTCGGAAGGACATATGGGAGGGCAAATCATTTAA | |
| | AACATCAGAATGAGTATTTGGTTTAGAGTTTGGCAACATATGCCCATTCTTCCG | |
| | CTTCCTCGCTCACTGACTCGCTGCGCTCGGTCGTTCGGCTGCGGCGAGCGGTA | |
| | TCAGCTCACTCAAAGGCGGTAATACGGTTATCCACAGAATCAGGGGATAACGC | |
| | AGGAAAGAACATGTGAGCAAAAGGCCAGCAAAAGGCCAGGAACCGTAAAA | |
| | AGGCCGCGTTGCTGGCGTTTTTCCATAGGCTCCGCCCCCCTGACGAGCATCAC | |
| | AAAAATCGACGCTCAAGTCAGAGGTGGCGAAACCCGACAGGACTATAAAGAT | |
| | ACCAGGCGTTTCCCCCTGGAAGCTCCCTCGTGCGCTCTCCTGTTCCGACCCTG | |
| | CCGCTTACCGGATACCTGTCCGCCTTTCTCCCTTCGGGAAGCGTGGCGCTTTC | |
| | TCATAGCTCACGCTGTAGGTATCTCAGTTCGGTGTAGGTCGTTCGCTCCAAGC | |
| | TGGGCTGTGTGCACGAACCCCCCGTTCAGCCCGACCGCTGCGCCTTATCCGGT | |
| | AACTATCGTCTTGAGTCCAACCCGGTAAGACACGACTTATCGCCACTGGCAGC | |
| | AGCCACTGGTAACAGGATTAGCAGAGCGAGGTATGTAGGCGGTGCTACAGAG | |
| | TTCTTGAAGTGGTGGCCTAACTACGGCTACACTAGAAGAACAGTATTTGGTAT | |
| | CTGCGCTCTGCTGAAGCCAGTTACCTTCGGAAAAAGAGTTGGTAGCTCTTGAT | |
| | CCGGCAAACAAACCACCGCTGGTAGCGGTGGTTTTTTTGTTTGCAAGCAGCA | |
| | GATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGAG | |
| | ATTATCAAAAAGGATCTTCACCTAGATCCTTTTAAATTAAAAATGAAGTTTTAA | |
| | ATCAATCTAAAGTATATATGAGTAAACTTGGTCTGACAGTTACCAATGCTTAAT | |
| | CAGTGAGGCACCTATCTCAGCGATCTGTCTATTTCGTTCATCCATAGTTGCCTG | |
| | ACTCGGGGGGGGGGGGCGCTGAGGTCTGCCTCGTGAAGAAGGTGTTGCTGA | |
| | CTCATACCAGGCCTGAATCGCCCCATCATCCAGCCAGAAAGTGAGGGAGCCA | |
| | CGGTTGATGAGAGCTTTGTTGTAGGTGGACCAGTTGGTGATTTTGAACTTTTG | |
| | CTTTGCCACGGAACGGTCTGCGTTGTCGGGAAGATGCGTGATCTGATCCTTCA | |
| | ACTCAGCAAAAGTTCGATTTATTCAACAAAGCCGCCGTCCCGTCAAGTCAGC | |
| | GTAATGCTCTGCCAGTGTTACAACCAATTAACCAATTCTGATTAGAAAAACTC | |
| | ATCGAGCATCAAATGAAACTGCAATTTATTCATATCAGGATTATCAATACCATAT | |
| | TTTTGAAAAAGCCGTTTCTGTAATGAAGGAGAAAACTCACCGAGGCAGTTCC | |
| | ATAGGATGGCAAGATCCTGGTATCGGTCTGCGATTCCGACTCGTCCAACATCA | |
| | ATACAACCTATTAATTTCCCCTCGTCAAAAATAAGGTTATCAAGTGAGAAATCA | |
| | CCATGAGTGACGACTGAATCCGGTGAGAATGGCAAAAGCTTATGCATTTCTTT | |
| | CCAGACTTGTTCAACAGGCCAGCCATTACGCTCGTCATCAAAATCACTCGCAT | |
| | CAACCAAACCGTTATTCATTCGTGATTGCGCCTGAGCGAGACGAAATACGCGA | |
| | TCGCTGTTAAAAGGACAATTACAAACAGGAATCGAATGCAACCGGCGCAGGA | |
| | ACACTGCCAGCGCATCAACAATATTTTCACCTGAATCAGGATATTCTTCTAATA | |
| | CCTGGAATGCTGTTTTCCCGGGGATCGCAGTGGTGAGTAACCATGCATCATCA | |
| | GGAGTACGGATAAAATGCTTGATGGTCGGAAGAGGCATAAATTCCGTCAGCC | |
| | AGTTTAGTCTGACCATCTCATCTGTAACATCATTGGCAACGCTACCTTTGCCAT | |
| | GTTTCAGAAACAACTCTGGCGCATCGGGCTTCCCATACAATCGATAGATTGTC | |
| | GCACCTGATTGCCCGACATTATCGCGAGCCCATTTATACCCATATAAATCAGCA | |
| | TCCATGTTGGAATTTAATCGCGGCCTCGAGCAAGACGTTTCCCGTTGAATATG | |
| | GCTCATAACACCCCTTGTATTACTGTTTATGTAAGCAGACAGTTTTATTGTTCAT | |
| | GATGATATATTTTTATCTTGTGCAATGTAACATCAGAGATTTTGAGACACAACG | |
| | TGGCTTTCCCCCCCCCCCCATTATTGAAGCATTTATCAGGGTTATTGTCTCATG | |
| | AGCGGATACATATTTGAATGTATTTAGAAAAATAAACAAATAGGGGTTCCGCG | |
| | CACATTTCCCCGAAAAGTGCCACCTGACGTCTAAGAAACCATTATTATCATGA | |
| | CATTAACCTATAAAAATAGGCGTATCACGAGGCCCTTTCGTCTCGCGCGTTTCG | |
| | GTGATGACGGTGAAAACCTCTGACACATGCAGCTCCCGGAGACGGTCACAGC | |
| | TTGTCTGTAAGCGGATGCCGGGAGCAGACAAGCCCGTCAGGGCGCGTCAGCG | |
| | GGTGTTGGCGGGTGTCGGGGCTGGCTTAACTATGCGGCATCAGAGCAGATTG | |
| | TACTGAGAGTGCACCATATGCGGTGTGAAATACCGCACAGATGCGTAAGGAG | |
| | AAAATACCGCATCAGATTGGCTAT | |
| 20 | TGGCCATTGCATACGTTGTATCCATATCATAATATGTACATTTATATTGGCTCATG | pGBI-2014 (triple |
| | TCCAACATTACCGCCATGTTGACATTGATTATTGACTAGTTATTAATAGTAATCA | knockdown bi- |
| | ATTACGGGGTCATTAGTTCATAGCCCATATATGGAGTTCCGCGTTACATAACTTA | shRNA construct #4 |
| | CGGTAAATGGCCCGCCTGGCTGACCGCCCAACGACCCCCGCCCATTGACGTC | targeting Type 1, |
| | AATAATGACGTATGTTCCCATAGTAACGCCAATAGGGACTTTCCATTGACGTCA | Type 2, and the rare |
| | ATGGGTGGAGTATTTACGGTAAACTGCCCACTTGGCAGTACATCAAGTGTATC | EWSR1-ATF1 |
| | ATATGCCAAGTACGCCCCCTATTGACGTCAATGACGGTAAATGGCCCGCCTGG | fusion genes; bi- |
| | CATTATGCCCAGTACATGACCTTATGGGACTTTCCTACTTGGCAGTACATCTAC | shRNA sequence in |
| | GTATTAGTCATCGCTATTACCATGGTGATGCGGTTTTGGCAGTACATCAATGGG | bold |
| | CGTGGATAGCGGTTTGACTCACGGGGATTTCCAAGTCTCCACCCCATTGACGT | |
| | CAATGGGAGTTTGTTTTGGCACCAAAATCAACGGGACTTTCCAAAATGTCGTA | |
| | ACAACTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACGCCATC | |
| | CACGCTGTTTTGACCTCCATAGAAGACACCGGGACCGATCCAGCCTCCGCGG | |
| | CCGGGAACGGTGCATTGGAACGCGGATTCCCCGTGCCAAGAGTGACTCACCG | |
| | TCGTCGACTCTTCCCCATTAGGGATTATGCTGAATTTGTATGGTTTATAGT | |
| | TGTTAGAGTTTGAGGTGTTAATTCTAATTATCTATTTCAAATTTAGCAGGA | |
| | AAAAAGAGAACATCACCTTGTAAAACTGAAGATTGTGACCAGTCAGAATA | |
| | ATGTCCGTGGAATGGTTTGATGCTGCATGATATGTGCATCTTGCAGCATC | |
| | AAACCATTCCACGGCATTATGGTGACAGCTGCCTCGGGAAGCCAAGTTG | |
| | GGCTTTAAAGTGCAGGGCCTGCTGATGTTGAGTGCTTTTTGTTCCCATGT | |
| | AATGACCTGATGATACATGAAGTAGATTAGCATCTTGCAGCATCAAACCA | |
| | TTCCACGGCATAAGAAGTTATGTATTCATCCAATAATTCAAGCCAAGCAA | |
| | GTATATAGGTGTTTTAATAGTTTTTGTTTGCAGTCCTCTGTTGAGGACGC | |
| | GGTGGAATGGGAAAAAGAAGAATGTAGTTTTTCCCATTCCACCGCGTCC | |
| | TCTGGTGGCCTGCTATTTCCTTCAAATGAATGATTTTTACTAATTTTGTGT | |
| | ACTTTTATTGTGTCGATGTAGAATCTGCCTGGTCTATCTGATGTGACAGC | |
| | TTCTGTAGCACGAGTTAGCGAGTGAATGGTCAAATGTTTAGTTATCTTTT | |
| | TCCCATTCCACCGCGTCCTCTACTGCTAGCTGTAGAACTCCAGCTTCGGC | |
| | CTGTCGCCCAATCAAACTGTCCTGTTACTGAACACTGTTCTATGGTTAGC | |
| | AGCTACGGGCAGCAGATTGCTGTGTGATATTCTGCGCAATCTGCTGCCC | |
| | GTAGCTGCTCTGTGGTAGTGAAAAGTCTGTAGAAAAGTAAGGGAAACTC | |
| | AAACCCCTTTCTACACAGCATACACATACAGCATCATGCGTGTTTCTGTAT | |
| | GGGCAATCTGCTGCCCGTAGCTGCTTTGAGTTTGGTGGGGATTGTGACC | |
| | AGAAGATTTTGAAAATTAAATATTACTGAAGATTTCGACTTCCACTGTTAA | |
| | ATGTACAAGATACATGAAATATTAAAGAAAATGTGTAACTTTTTGTGTAAA | |
| | TACATCTTGTCTTGTTTTCATTCAAAAACATTTCACTTTTGGGGTTGCGT | |
| | GTCAGATTTGGCAGTATAAATTCTGGCTATATTTTTTGCGGCCGCGGATCC | |
| | AGATCTTTTTCCCTCTGCCAAAAATTATGGGGACATCATGAAGCCCCTTGAGC | |
| | ATCTGACTTCTGGCTAATAAAGGAAATTTATTTTCATTGCAATAGTGTGTTGGA | |
| | ATTTTTTGTGTCTCTCACTCGGAAGGACATATGGGAGGGCAAATCATTTAAAA | |
| | CATCAGAATGAGTATTTGGTTTAGAGTTTGGCAACATATGCCCATTCTTCCGCT | |
| | TCCTCGCTCACTGACTCGCTGCGCTCGGTCGTTCGGCTGCGGCGAGCGGTATC | |
| | AGCTCACTCAAAGGCGGTAATACGGTTATCCACAGAATCAGGGGATAACGCA | |
| | GGAAAGAACATGTGAGCAAAAGGCCAGCAAAAGGCCAGGAACCGTAAAAA | |
| | GGCCGCGTTGCTGGCGTTTTTCCATAGGCTCCGCCCCCCTGACGAGCATCACA | |
| | AAAATCGACGCTCAAGTCAGAGGTGGCGAAACCCGACAGGACTATAAAGATA | |
| | CCAGGCGTTTCCCCCTGGAAGCTCCCTCGTGCGCTCTCCTGTTCCGACCCTGC | |
| | CGCTTACCGGATACCTGTCCGCCTTTCTCCCTTCGGGAAGCGTGGCGCTTTCT | |
| | CATAGCTCACGCTGTAGGTATCTCAGTTCGGTGTAGGTCGTTCGCTCCAAGCT | |
| | GGGCTGTGTGCACGAACCCCCCGTTCAGCCCGACCGCTGCGCCTTATCCGGT | |
| | AACTATCGTCTTGAGTCCAACCCGGTAAGACACGACTTATCGCCACTGGCAGC | |
| | AGCCACTGGTAACAGGATTAGCAGAGCGAGGTATGTAGGCGGTGCTACAGAG | |
| | TTCTTGAAGTGGTGGCCTAACTACGGCTACACTAGAAGAACAGTATTTGGTAT | |
| | CTGCGCTCTGCTGAAGCCAGTTACCTTCGGAAAAAGAGTTGGTAGCTCTTGAT | |
| | CCGGCAAACAAACCACCGCTGGTAGCGGTGGTTTTTTTGTTTGCAAGCAGCA | |
| | GATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGAG | |
| | ATTATCAAAAAGGATCTTCACCTAGATCCTTTTAAATTAAAAATGAAGTTTTAA | |
| | ATCAATCTAAAGTATATATGAGTAAACTTGGTCTGACAGTTACCAATGCTTAAT | |
| | CAGTGAGGCACCTATCTCAGCGATCTGTCTATTTCGTTCATCCATAGTTGCCTG | |
| | ACTCGGGGGGGGGGGGCGCTGAGGTCTGCCTCGTGAAGAAGGTGTTGCTGA | |
| | CTCATACCAGGCCTGAATCGCCCCATCATCCAGCCAGAAAGTGAGGGAGCCA | |
| | CGGTTGATGAGAGCTTTGTTGTAGGTGGACCAGTTGGTGATTTTGAACTTTTG | |
| | CTTTGCCACGGAACGGTCTGCGTTGTCGGGAAGATGCGTGATCTGATCCTTCA | |
| | ACTCAGCAAAAGTTCGATTTATTCAACAAAGCCGCCGTCCCGTCAAGTCAGC | |
| | GTAATGCTCTGCCAGTGTTACAACCAATTAACCAATTCTGATTAGAAAAACTC | |
| | ATCGAGCATCAAATGAAACTGCAATTTATTCATATCAGGATTATCAATACCATAT | |
| | TTTTGAAAAAGCCGTTTCTGTAATGAAGGAGAAAACTCACCGAGGCAGTTCC | |
| | ATAGGATGGCAAGATCCTGGTATCGGTCTGCGATTCCGACTCGTCCAACATCA | |
| | ATACAACCTATTAATTTCCCCTCGTCAAAAATAAGGTTATCAAGTGAGAAATCA | |
| | CCATGAGTGACGACTGAATCCGGTGAGAATGGCAAAAGCTTATGCATTTCTTT | |
| | CCAGACTTGTTCAACAGGCCAGCCATTACGCTCGTCATCAAAATCACTCGCAT | |
| | CAACCAAACCGTTATTCATTCGTGATTGCGCCTGAGCGAGACGAAATACGCGA | |
| | TCGCTGTTAAAAGGACAATTACAAACAGGAATCGAATGCAACCGGCGCAGGA | |
| | ACACTGCCAGCGCATCAACAATATTTTCACCTGAATCAGGATATTCTTCTAATA | |
| | CCTGGAATGCTGTTTTCCCGGGGATCGCAGTGGTGAGTAACCATGCATCATCA | |
| | GGAGTACGGATAAAATGCTTGATGGTCGGAAGAGGCATAAATTCCGTCAGCC | |
| | AGTTTAGTCTGACCATCTCATCTGTAACATCATTGGCAACGCTACCTTTGCCAT | |
| | GTTTCAGAAACAACTCTGGCGCATCGGGCTTCCCATACAATCGATAGATTGTC | |
| | GCACCTGATTGCCCGACATTATCGCGAGCCCATTTATACCCATATAAATCAGCA | |
| | TCCATGTTGGAATTTAATCGCGGCCTCGAGCAAGACGTTTCCCGTTGAATATG | |
| | GCTCATAACACCCCTTGTATTACTGTTTATGTAAGCAGACAGTTTTATTGTTCAT | |
| | GATGATATATTTTTATCTTGTGCAATGTAACATCAGAGATTTTGAGACACAACG | |
| | TGGCTTTCCCCCCCCCCCCATTATTGAAGCATTTATCAGGGTTATTGTCTCATG | |
| | AGCGGATACATATTTGAATGTATTTAGAAAAATAAACAAATAGGGGTTCCGCG | |
| | CACATTTCCCCGAAAAGTGCCACCTGACGTCTAAGAAACCATTATTATCATGA | |
| | CATTAACCTATAAAAATAGGCGTATCACGAGGCCCTTTCGTCTCGCGCGTTTCG | |
| | GTGATGACGGTGAAAACCTCTGACACATGCAGCTCCCGGAGACGGTCACAGC | |
| | TTGTCTGTAAGCGGATGCCGGGAGCAGACAAGCCCGTCAGGGCGCGTCAGCG | |
| | GGTGTTGGCGGGTGTCGGGGCTGGCTTAACTATGCGGCATCAGAGCAGATTG | |
| | TACTGAGAGTGCACCATATGCGGTGTGAAATACCGCACAGATGCGTAAGGAG | |
| | AAAATACCGCATCAGATTGGCTAT | |
| 21 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTCGTGGAATGGTT | EWSR1-ATF1 rare |
| | TGATGCTGCATAGATATGTGCATCTATGCAGCATCAAACCATTCCACGCATTAT | fusion gene (in |
| | GGTGACAGCTGCCTCGGGAAGCCAAGTTGGGCTTTAAAGTGCAGGGCCTGCT | pGBI-2003) |
| | GATGTTGAGTGCTTTTTGTTCCGTGTCATGCAGAGATGCTTGATAGTGAAGTA | |
| | GATTAGCATCTATGCAGCATCAAACCATTCCACGCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 22 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTCCGTGGAATGGT | EWSR1-ATF1 rare |
| | TTGATGCTGCAAGATATGTGCATCTTGCAGCATCAAACCATTCCACGGCATTAT | fusion gene (in |
| | GGTGACAGCTGCCTCGGGAAGCCAAGTTGGGCTTTAAAGTGCAGGGCCTGCT | pGBI-2004) |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGT | |
| 23 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTGCCGTGGAATG | EWSR1-ATF1 rare |
| | GTTTGATGCTGCAGATATGTGCATCTGCAGCATCAAACCATTCCACGGCCATTA | fusion gene (in |
| | TGGTGACAGCTGCCTCGGGAAGCCAAGTTGGGCTTTAAAGTGCAGGGCCTGC | pGBI-2005) |
| | TGATGTTGAGTGCTTTTTGTTCGCTATGTCATACATTGATGACGCAGTGAAGTA | |
| | GATTAGCATCTGCAGCATCAAACCATTCCACGGCCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 24 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTTGCCGTGGAATG | EWSR1-ATF1 rare |
| | GTTTGATGCTGAGATATGTGCATCTCAGCATCAAACCATTCCACGGCACATTAT | fusion gene (in |
| | GGTGACAGCTGCCTCGGGAAGCCAAGTTGGGCTTTAAAGTGCAGGGCCTGCT | pGBI-2006) |
| | GATGTTGAGTGCTTTTTGTTCTGATGTGTAACAATTTGACGATGAGTGAAGTA | |
| | GATTAGCATCTCAGCATCAAACCATTCCACGGCACATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 25 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTCTGCCGTGGAAT | EWSR1-ATF1 rare |
| | GGTTTGATGCTAGATATGTGCATCTAGCATCAAACCATTCCACGGCAGCATTAT | fusion gene (in |
| | GGTGACAGCTGCCTCGGGAAGCCAAGTTGGGCTTTAAAGTGCAGGGCCTGCT | pGBI-2007) |
| | GATGTTGAGTGCTTTTTGTTCCTGATGTGGATCAGTTTGACACTAGTGAAGTA | |
| | GATTAGCATCTAGCATCAAACCATTCCACGGCAGCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 26 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTTGCCGTGGAATG | EWSR1-ATF1 rare |
| | GTTTGATGCTGAGATATGTGCATCTCAGCATCAAACCATTCCACGGCACATTAT | fusion gene (in |
| | GGTGACAGCTGCCTCGGGAAGCCAAGTTGGGCTTTAAAGTGCAGGGCCTGCT | pGBI-2008) |
| | GATGTTGAGTGCTTTTTGTTCTGATGTGTAACAATTTGACGATGAGTGAAGTA | |
| | GATTAGCATCTCAGCATCAAACCATTCCACGGCACATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 27 | TCTTCCCCATTAGGGATTATGCTGAATTTGTATGGTTTATAGTTGTTAGAGTTTG | bi-shRNA sequence |
| | AGGTGTTAATTCTAATTATCTATTTCAAATTTAGCAGGAAAAAAGAGAACATCA | targeting Type 1, |
| | CCTTGTAAAACTGAAGATTGTGACCAGTCAGAATAATGTCCGTGGAATGGTTT | Type 2, and |
| | GATGCTGCATGATATGTGCATCTTGCAGCATCAAACCATTCCACGGCATTATGG | EWSR1-ATF1 rare |
| | TGACAGCTGCCTCGGGAAGCCAAGTTGGGCTTTAAAGTGCAGGGCCTGCTGA | fusion genes (in |
| | TGTTGAGTGCTTTTTGTTCCCATGTAATGACCTGATGATACATGAAGTAGATTA | pGBI-2011) |
| | GCATCTTGCAGCATCAAACCATTCCACGGCATAAGAAGTTATGTATTCATCCAA | |
| | TAATTCAAGCCAAGCAAGTATATAGGTGTTTTAATAGTTTTTGTTTGCAGTCCT | |
| | CTGTTAGGACGCGGTGGAATGGGAAAAAAGAAGAATGTAGTTTTTTCCCATT | |
| | CCACCGCGTCCTTGGTGGCCTGCTATTTCCTTCAAATGAATGATTTTTACTAAT | |
| | TTTGTGTACTTTTATTGTGTCGATGTAGAATCTGCCTGGTCTATCTGATGTGAC | |
| | AGCTTCTGTAGCACAGGACGATGAACAATGGGTGAAATGTTTAGTTATCTTTT | |
| | TTCCCATTCCACCGCGTCCTTACTGCTAGCTGTAGAACTCCAGCTTCGGCCTG | |
| | TCGCCCAATCAAACTGTCCTGTTACTGAACACTGTTCTATGGTTGCAGCTACG | |
| | GGCAGCAGATTGCCTGTGTGATATTCTGCGGCAATCTGCTGCCCGTAGCTGCC | |
| | TGTGGTAGTGAAAAGTCTGTAGAAAAGTAAGGGAAACTCAAACCCCTTTCTA | |
| | CACGCAGACACGAGTAGCAGATCACCGTGTTTCTGTATGGGGCAATCTGCTGC | |
| | CCGTAGCTGCTTGAGTTTGGTGGGGATTGTGACCAGAAGATTTTGAAAATTAA | |
| | ATATTACTGAAGATTTCGACTTCCACTGTTAAATGTACAAGATACATGAAATAT | |
| | TAAAGAAAATGTGTAACTTTTTGTGTAAATACATCTTGTCTTGTTTTCATTCAA | |
| | AAACATTTCACTTTTGGGGTTGCGTGTCAGATTTGGCAGTATAAATTCTGGCTA | |
| | TATTTTTT | |
| 28 | TCTTCCCCATTAGGGATTATGCTGAATTTGTATGGTTTATAGTTGTTAGAGTTTG | bi-shRNA sequence |
| | AGGTGTTAATTCTAATTATCTATTTCAAATTTAGCAGGAAAAAAGAGAACATCA | targeting Type 1, |
| | CCTTGTAAAACTGAAGATTGTGACCAGTCAGAATAATGTCCGTGGAATGGTTT | Type 2, and |
| | GATGCTGCATGATATGTGCATCTTGCAGCATCAAACCATTCCACGGCATTATGG | EWSR1-ATF1 rare |
| | TGACAGCTGCCTCGGGAAGCCAAGTTGGGCTTTAAAGTGCAGGGCCTGCTGA | fusion genes (in |
| | TGTTGAGTGCTTTTTGTTCCCATGTAATGACCTGATGATACATGAAGTAGATTA | pGBI-2012) |
| | GCATCTTGCAGCATCAAACCATTCCACGGCATAAGAAGTTATGTATTCATCCAA | |
| | TAATTCAAGCCAAGCAAGTATATAGGTGTTTTAATAGTTTTTGTTTGCAGTCCT | |
| | CTGTTAGGACGCGGTGGAATGGGAAAAAAGAAGAATGTAGTTTTTTCCCATT | |
| | CCACCGCGTCCTTGGTGGCCTGCTATTTCCTTCAAATGAATGATTTTTACTAAT | |
| | TTTGTGTACTTTTATTGTGTCGATGTAGAATCTGCCTGGTCTATCTGATGTGAC | |
| | AGCTTCTGTAGCACAGGACGATGAACAATGGGTGAAATGTTTAGTTATCTTTT | |
| | TTCCCATTCCACCGCGTCCTTACTGCTAGCTGTAGAACTCCAGCTTCGGCCTG | |
| | TCGCCCAATCAAACTGTCCTGTTACTGAACACTGTTCTATGGTTGCAGCTACG | |
| | GGCAGCAGATTGCCTGTGTGATATTCTGCGGCAATCTGCTGCCCGTAGCTGCC | |
| | TGTGGTAGTGAAAAGTCTGTAGAAAAGTAAGGGAAACTCAAACCCCTTTCTA | |
| | CACGCAGACACGAGTAGCAGATCACCGTGTTTCTGTATGGGGCAATCTGCTGC | |
| | CCGTAGCTGCTTGAGTTTGGTGGGGATTGTGACCAGAAGATTTTGAAAATTAA | |
| | ATATTACTGAAGATTTCGACTTCCACTGTTAAATGTACAAGATACATGAAATAT | |
| | TAAAGAAAATGTGTAACTTTTTGTGTAAATACATCTTGTCTTGTTTTCATTCAA | |
| | AAACATTTCACTTTTGGGGTTGCGTGTCAGATTTGGCAGTATAAATTCTGGCTA | |
| | TATTTTTT | |
| 29 | TCTTCCCCATTAGGGATTATGCTGAATTTGTATGGTTTATAGTTGTTAGAGTTTG | bi-shRNA sequence |
| | AGGTGTTAATTCTAATTATCTATTTCAAATTTAGCAGGAAAAAAGAGAACATCA | targeting Type 1, |
| | CCTTGTAAAACTGAAGATTGTGACCAGTCAGAATAATGTCCGTGGAATGGTTT | Type 2, and |
| | GATGCTGCATGATATGTGCATCTTGCAGCATCAAACCATTCCACGGCATTATGG | EWSR1-ATF1 rare |
| | TGACAGCTGCCTCGGGAAGCCAAGTTGGGCTTTAAAGTGCAGGGCCTGCTGA | fusion genes (in |
| | TGTTGAGTGCTTTTTGTTCCCATGTAATGACCTGATGATACATGAAGTAGATTA | pGBI-2013) |
| | GCATCTTGCAGCATCAAACCATTCCACGGCATAAGAAGTTATGTATTCATCCAA | |
| | TAATTCAAGCCAAGCAAGTATATAGGTGTTTTAATAGTTTTTGTTTGCAGTCCT | |
| | CTGTTGAGGACGCGGTGGAATGGGAAAAAGAAGAATGTAGTTTTTCCCATTC | |
| | CACCGCGTCCTCTGGTGGCCTGCTATTTCCTTCAAATGAATGATTTTTACTAAT | |
| | TTTGTGTACTTTTATTGTGTCGATGTAGAATCTGCCTGGTCTATCTGATGTGAC | |
| | AGCTTCTGTAGCACGAGTTAGCGAGTGAATGGTCAAATGTTTAGTTATCTTTTT | |
| | CCCATTCCACCGCGTCCTCTACTGCTAGCTGTAGAACTCCAGCTTCGGCCTGT | |
| | CGCCCAATCAAACTGTCCTGTTACTGAACACTGTTCTATGGTTAGCAGCTACG | |
| | GGCAGCAGATTGCTGTGTGATATTCTGCGCAATCTGCTGCCCGTAGCTGCTCT | |
| | GTGGTAGTGAAAAGTCTGTAGAAAAGTAAGGGAAACTCAAACCCCTTTCTAC | |
| | ACAGCATACACATACAGCATCATGCGTGTTTCTGTATGGGCAATCTGCTGCCCG | |
| | TAGCTGCTTTGAGTTTGGTGGGGATTGTGACCAGAAGATTTTGAAAATTAAAT | |
| | ATTACTGAAGATTTCGACTTCCACTGTTAAATGTACAAGATACATGAAATATTA | |
| | AAGAAAATGTGTAACTTTTTGTGTAAATACATCTTGTCTTGTTTTCATTCAAAA | |
| | ACATTTCACTTTTGGGGTTGCGTGTCAGATTTGGCAGTATAAATTCTGGCTATA | |
| | TTTTTT | |
| 30 | TCTTCCCCATTAGGGATTATGCTGAATTTGTATGGTTTATAGTTGTTAGAGTTTG | bi-shRNA sequence |
| | AGGTGTTAATTCTAATTATCTATTTCAAATTTAGCAGGAAAAAAGAGAACATCA | targeting Type 1, |
| | CCTTGTAAAACTGAAGATTGTGACCAGTCAGAATAATGTCCGTGGAATGGTTT | Type 2, and |
| | GATGCTGCATGATATGTGCATCTTGCAGCATCAAACCATTCCACGGCATTATGG | EWSR1-ATF1 rare |
| | TGACAGCTGCCTCGGGAAGCCAAGTTGGGCTTTAAAGTGCAGGGCCTGCTGA | fusion genes (in |
| | TGTTGAGTGCTTTTTGTTCCCATGTAATGACCTGATGATACATGAAGTAGATTA | pGBI-2014) |
| | GCATCTTGCAGCATCAAACCATTCCACGGCATAAGAAGTTATGTATTCATCCAA | |
| | TAATTCAAGCCAAGCAAGTATATAGGTGTTTTAATAGTTTTTGTTTGCAGTCCT | |
| | CTGTTGAGGACGCGGTGGAATGGGAAAAAGAAGAATGTAGTTTTTCCCATTC | |
| | CACCGCGTCCTCTGGTGGCCTGCTATTTCCTTCAAATGAATGATTTTTACTAAT | |
| | TTTGTGTACTTTTATTGTGTCGATGTAGAATCTGCCTGGTCTATCTGATGTGAC | |
| | AGCTTCTGTAGCACGAGTTAGCGAGTGAATGGTCAAATGTTTAGTTATCTTTTT | |
| | CCCATTCCACCGCGTCCTCTACTGCTAGCTGTAGAACTCCAGCTTCGGCCTGT | |
| | CGCCCAATCAAACTGTCCTGTTACTGAACACTGTTCTATGGTTAGCAGCTACG | |
| | GGCAGCAGATTGCTGTGTGATATTCTGCGCAATCTGCTGCCCGTAGCTGCTCT | |
| | GTGGTAGTGAAAAGTCTGTAGAAAAGTAAGGGAAACTCAAACCCCTTTCTAC | |
| | ACAGCATACACATACAGCATCATGCGTGTTTCTGTATGGGCAATCTGCTGCCCG | |
| | TAGCTGCTTTGAGTTTGGTGGGGATTGTGACCAGAAGATTTTGAAAATTAAAT | |
| | ATTACTGAAGATTTCGACTTCCACTGTTAAATGTACAAGATACATGAAATATTA | |
| | AAGAAAATGTGTAACTTTTTGTGTAAATACATCTTGTCTTGTTTTCATTCAAAA | |
| | ACATTTCACTTTTGGGGTTGCGTGTCAGATTTGGCAGTATAAATTCTGGCTATA | |
| | TTTTTT | |
| 31 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 |
| | CACCTTGTAAAACTGAAGATTGTGACCAGTCAGAATAATGTAGGACGCGGTG | EWSR1-ATF1 |
| | GAATGGGAAAAAAGATATGTGCATCTTTTTTCCCATTCCACCGCGTCCTCATTA | fusion gene |
| | TGGTGACAGCTGCCTCGGGAAGCCAAGTTGGGCTTTAAAGTGCAGGGCCTGC | |
| | TGATGTTGAGTGCTTTTTGTTCAGGACGATGAACAATGGGTGAAAAGTGAAG | |
| | TAGATTAGCATCTTTTTTCCCATTCCACCGCGTCCTCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGT | |
| 32 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 2 |
| | CACCTTGTAAAACTGAAGATTGTGACCAGTCAGAATAATGTGCAGCTACGGG | EWSR1-ATF1 |
| | CAGCAGATTGCCAGATATGTGCATCTGGCAATCTGCTGCCCGTAGCTGCCATTA | fusion gene |
| | TGGTGACAGCTGCCTCGGGAAGCCAAGTTGGGCTTTAAAGTGCAGGGCCTGC | |
| | TGATGTTGAGTGCTTTTTGTTCGCAGACACGAGTAGCAGATCACCAGTGAAGT | |
| | AGATTAGCATCTGGCAATCTGCTGCCCGTAGCTGCCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGT | |
| 33 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 3 |
| | CACCTTGTAAAACTGAAGATTGTGACCAGTCAGAATAATGTGACCAGATCTTG | EWSR1-ATF1 |
| | ATCTAGTTGCCAGATATGTGCATCTGGCAACTAGATCAAGATCTGGTCCATTAT | fusion gene |
| | GGTGACAGCTGCCTCGGGAAGCCAAGTTGGGCTTTAAAGTGCAGGGCCTGCT | |
| | GATGTTGAGTGCTTTTTGTTCGACAGGATCGATATCTAGTCACCAGTGAAGTA | |
| | GATTAGCATCTGGCAACTAGATCAAGATCTGGTCCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGT | |
| 34 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 4 |
| | CACCTTGTAAAACTGAAGATTGTGACCAGTCAGAATAATGTGCAGCTACGGG | EWSR1-ATF1 |
| | CAGCAGAAGAAGAGATATGTGCATCTCTTCTTCTGCTGCCCGTAGCTGCCATT | fusion gene |
| | ATGGTGACAGCTGCCTCGGGAAGCCAAGTTGGGCTTTAAAGTGCAGGGCCTG | |
| | CTGATGTTGAGTGCTTTTTGTTCGCACATACGATGAGCAGACTAAGAGTGAAG | |
| | TAGATTAGCATCTCTTCTTCTGCTGCCCGTAGCTGCCATAAGAAGTTATGTATT | |
| | CATCCAATAATTCAAGCCAAGCAAGTATATAGGTGTTTTAATAGTTTTTGTTTT | |
| | GGGGATTGTGACCAGAAGATTTTGAAAATTAAATATTACTGAAGATTTCGACT | |
| | TCCACTGTTAAATGTACAAGATACATGAAATATTAAAGAAAATGTGTAACTTTT | |
| | TGTGTAAATACATCTTGT | |
| 35 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 and |
| | CACCTTGTAAAACTGAAGATTGTGACCAGTCAGAATAATGTAGGACGCGGTG | Type 2 EWSR1- |
| | GAATGGGAAAAAAGATATGTGCATCTTTTTTCCCATTCCACCGCGTCCTCATTA | ATF1 fusion genes |
| | TGGTGACAGCTGCCTCGGGAAGCCAAGTTGGGCTTTAAAGTGCAGGGCCTGC | |
| | TGATGTTGAGTGCTTTTTGTTCAGGACGATGAACAATGGGTGAAAAGTGAAG | |
| | TAGATTAGCATCTTTTTTCCCATTCCACCGCGTCCTCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGCAGCTACGGGCAGCAGATTGCCAGATATGTGCATCTGGCAATCTGCT | |
| | GCCCGTAGCTGCCATTATGGTGACAGCTGCCTCGGGAAGCCAAGTTGGGCTTT | |
| | AAAGTGCAGGGCCTGCTGATGTTGAGTGCTTTTTGTTCGCAGACACGAGTAG | |
| | CAGATCACCAGTGAAGTAGATTAGCATCTGGCAATCTGCTGCCCGTAGCTGCC | |
| | ATAAGAAGTTATGTATTCATCCAATAATTCAAGCCAAGCAAGTATATAGGTGTT | |
| | TTAATAGTTTTTGTTTTGGGGATTGTGACCAGAAGATTTTGAAAATTAAATATT | |
| | ACTGAAGATTTCGACTTCCACTGTTAAATGTACAAGATACATGAAATATTAAAG | |
| | AAAATGTGTAACTTTTTGTGTAAATACATCTTGT | |
| 36 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 and |
| | CACCTTGTAAAACTGAAGATTGTGACCAGTCAGAATAATGTAGGACGCGGTG | Type 3 EWSR1- |
| | GAATGGGAAAAAAGATATGTGCATCTTTTTTCCCATTCCACCGCGTCCTCATTA | ATF1 fusion genes |
| | TGGTGACAGCTGCCTCGGGAAGCCAAGTTGGGCTTTAAAGTGCAGGGCCTGC | |
| | TGATGTTGAGTGCTTTTTGTTCAGGACGATGAACAATGGGTGAAAAGTGAAG | |
| | TAGATTAGCATCTTTTTTCCCATTCCACCGCGTCCTCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGACCAGATCTTGATCTAGTTGCCAGATATGTGCATCTGGCAACTAGATC | |
| | AAGATCTGGTCCATTATGGTGACAGCTGCCTCGGGAAGCCAAGTTGGGCTTTA | |
| | AAGTGCAGGGCCTGCTGATGTTGAGTGCTTTTTGTTCGACAGGATCGATATCT | |
| | AGTCACCAGTGAAGTAGATTAGCATCTGGCAACTAGATCAAGATCTGGTCCAT | |
| | AAGAAGTTATGTATTCATCCAATAATTCAAGCCAAGCAAGTATATAGGTGTTTT | |
| | AATAGTTTTTGTTTTGGGGATTGTGACCAGAAGATTTTGAAAATTAAATATTAC | |
| | TGAAGATTTCGACTTCCACTGTTAAATGTACAAGATACATGAAATATTAAAGA | |
| | AAATGTGTAACTTTTTGTGTAAATACATCTTGT | |
| 37 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 and |
| | CACCTTGTAAAACTGAAGATTGTGACCAGTCAGAATAATGTAGGACGCGGTG | Type 4 EWSR1- |
| | GAATGGGAAAAAAGATATGTGCATCTTTTTTCCCATTCCACCGCGTCCTCATTA | ATF1 fusion genes |
| | TGGTGACAGCTGCCTCGGGAAGCCAAGTTGGGCTTTAAAGTGCAGGGCCTGC | |
| | TGATGTTGAGTGCTTTTTGTTCAGGACGATGAACAATGGGTGAAAAGTGAAG | |
| | TAGATTAGCATCTTTTTTCCCATTCCACCGCGTCCTCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGCAGCTACGGGCAGCAGAAGAAGAGATATGTGCATCTCTTCTTCTGCT | |
| | GCCCGTAGCTGCCATTATGGTGACAGCTGCCTCGGGAAGCCAAGTTGGGCTTT | |
| | AAAGTGCAGGGCCTGCTGATGTTGAGTGCTTTTTGTTCGCACATACGATGAGC | |
| | AGACTAAGAGTGAAGTAGATTAGCATCTCTTCTTCTGCTGCCCGTAGCTGCCA | |
| | TAAGAAGTTATGTATTCATCCAATAATTCAAGCCAAGCAAGTATATAGGTGTTT | |
| | TAATAGTTTTTGTTTTGGGGATTGTGACCAGAAGATTTTGAAAATTAAATATTA | |
| | CTGAAGATTTCGACTTCCACTGTTAAATGTACAAGATACATGAAATATTAAAGA | |
| | AAATGTGTAACTTTTTGTGTAAATACATCTTGT | |
| 38 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 2 and |
| | CACCTTGTAAAACTGAAGATTGTGACCAGTCAGAATAATGTGCAGCTACGGG | Type 3 EWSR1- |
| | CAGCAGATTGCCAGATATGTGCATCTGGCAATCTGCTGCCCGTAGCTGCCATTA | ATF1 fusion genes |
| | TGGTGACAGCTGCCTCGGGAAGCCAAGTTGGGCTTTAAAGTGCAGGGCCTGC | |
| | TGATGTTGAGTGCTTTTTGTTCGCAGACACGAGTAGCAGATCACCAGTGAAGT | |
| | AGATTAGCATCTGGCAATCTGCTGCCCGTAGCTGCCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGACCAGATCTTGATCTAGTTGCCAGATATGTGCATCTGGCAACTAGATC | |
| | AAGATCTGGTCCATTATGGTGACAGCTGCCTCGGGAAGCCAAGTTGGGCTTTA | |
| | AAGTGCAGGGCCTGCTGATGTTGAGTGCTTTTTGTTCGACAGGATCGATATCT | |
| | AGTCACCAGTGAAGTAGATTAGCATCTGGCAACTAGATCAAGATCTGGTCCAT | |
| | AAGAAGTTATGTATTCATCCAATAATTCAAGCCAAGCAAGTATATAGGTGTTTT | |
| | AATAGTTTTTGTTTTGGGGATTGTGACCAGAAGATTTTGAAAATTAAATATTAC | |
| | TGAAGATTTCGACTTCCACTGTTAAATGTACAAGATACATGAAATATTAAAGA | |
| | AAATGTGTAACTTTTTGTGTAAATACATCTTGT | |
| 39 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 2 and |
| | CACCTTGTAAAACTGAAGATTGTGACCAGTCAGAATAATGTGCAGCTACGGG | Type 4 EWSR1- |
| | CAGCAGATTGCCAGATATGTGCATCTGGCAATCTGCTGCCCGTAGCTGCCATTA | ATF1 fusion genes |
| | TGGTGACAGCTGCCTCGGGAAGCCAAGTTGGGCTTTAAAGTGCAGGGCCTGC | |
| | TGATGTTGAGTGCTTTTTGTTCGCAGACACGAGTAGCAGATCACCAGTGAAGT | |
| | AGATTAGCATCTGGCAATCTGCTGCCCGTAGCTGCCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGCAGCTACGGGCAGCAGAAGAAGAGATATGTGCATCTCTTCTTCTGCT | |
| | GCCCGTAGCTGCCATTATGGTGACAGCTGCCTCGGGAAGCCAAGTTGGGCTTT | |
| | AAAGTGCAGGGCCTGCTGATGTTGAGTGCTTTTTGTTCGCACATACGATGAGC | |
| | AGACTAAGAGTGAAGTAGATTAGCATCTCTTCTTCTGCTGCCCGTAGCTGCCA | |
| | TAAGAAGTTATGTATTCATCCAATAATTCAAGCCAAGCAAGTATATAGGTGTTT | |
| | TAATAGTTTTTGTTTTGGGGATTGTGACCAGAAGATTTTGAAAATTAAATATTA | |
| | CTGAAGATTTCGACTTCCACTGTTAAATGTACAAGATACATGAAATATTAAAGA | |
| | AAATGTGTAACTTTTTGTGTAAATACATCTTGT | |
| 40 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type |
| | CACCTTGTAAAACTGAAGATTGTGACCAGTCAGAATAATGTGACCAGATCTTG | 3 and |
| | ATCTAGTTGCCAGATATGTGCATCTGGCAACTAGATCAAGATCTGGTCCATTAT | Type 4 EWSR1- |
| | GGTGACAGCTGCCTCGGGAAGCCAAGTTGGGCTTTAAAGTGCAGGGCCTGCT | ATF1 fusion genes |
| | GATGTTGAGTGCTTTTTGTTCGACAGGATCGATATCTAGTCACCAGTGAAGTA | |
| | GATTAGCATCTGGCAACTAGATCAAGATCTGGTCCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGTTTTCTTCCCCATTAGGGATTATGCTGAATTTGTATGGTTT | |
| | ATAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGCAGCTACGGGCAGCAGAAGAAGAGATATGTGCATCTCTTCTTCTGCTGC | |
| | CCGTAGCTGCCATTATGGTGACAGCTGCCTCGGGAAGCCAAGTTGGGCTTTAA | |
| | AGTGCAGGGCCTGCTGATGTTGAGTGCTTTTTGTTCGCACATACGATGAGCAG | |
| | ACTAAGAGTGAAGTAGATTAGCATCTCTTCTTCTGCTGCCCGTAGCTGCCATA | |
| | AGAAGTTATGTATTCATCCAATAATTCAAGCCAAGCAAGTATATAGGTGTTTTA | |
| | ATAGTTTTTGTTTTGGGGATTGTGACCAGAAGATTTTGAAAATTAAATATTACT | |
| | GAAGATTTCGACTTCCACTGTTAAATGTACAAGATACATGAAATATTAAAGAA | |
| | AATGTGTAACTTTTTGTGTAAATACATCTTGT | |
| 41 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 and |
| | CACCTTGTAAAACTGAAGATTGTGACCAGTCAGAATAATGTCCGTGGAATGGT | EWSR1-ATF1 rare |
| | TTGATGCTGCAAGATATGTGCATCTTGCAGCATCAAACCATTCCACGGCATTAT | fusion genes |
| | GGTGACAGCTGCCTCGGGAAGCCAAGTTGGGCTTTAAAGTGCAGGGCCTGCT | |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGTTTTCTTCCCCATTAGGGATTATGCTGAATTTGTATGGTTTA | |
| | TAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTAGGACGCGGTGGAATGGGAAAAAAGATATGTGCATCTTTTTTCCCATTCC | |
| | ACCGCGTCCTCATTATGGTGACAGCTGCCTCGGGAAGCCAAGTTGGGCTTTAA | |
| | AGTGCAGGGCCTGCTGATGTTGAGTGCTTTTTGTTCAGGACGATGAACAATG | |
| | GGTGAAAAGTGAAGTAGATTAGCATCTTTTTTCCCATTCCACCGCGTCCTCATA | |
| | AGAAGTTATGTATTCATCCAATAATTCAAGCCAAGCAAGTATATAGGTGTTTTA | |
| | ATAGTTTTTGTTTTGGGGATTGTGACCAGAAGATTTTGAAAATTAAATATTACT | |
| | GAAGATTTCGACTTCCACTGTTAAATGTACAAGATACATGAAATATTAAAGAA | |
| | AATGTGTAACTTTTTGTGTAAATACATCTTGT | |
| 42 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 2 and |
| | CACCTTGTAAAACTGAAGATTGTGACCAGTCAGAATAATGTCCGTGGAATGGT | EWSR1-ATF1 rare |
| | TTGATGCTGCAAGATATGTGCATCTTGCAGCATCAAACCATTCCACGGCATTAT | fusion genes |
| | GGTGACAGCTGCCTCGGGAAGCCAAGTTGGGCTTTAAAGTGCAGGGCCTGCT | |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGTTTTCTTCCCCATTAGGGATTATGCTGAATTTGTATGGTTTA | |
| | TAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGCAGCTACGGGCAGCAGATTGCCAGATATGTGCATCTGGCAATCTGCTGC | |
| | CCGTAGCTGCCATTATGGTGACAGCTGCCTCGGGAAGCCAAGTTGGGCTTTAA | |
| | AGTGCAGGGCCTGCTGATGTTGAGTGCTTTTTGTTCGCAGACACGAGTAGCA | |
| | GATCACCAGTGAAGTAGATTAGCATCTGGCAATCTGCTGCCCGTAGCTGCCAT | |
| | AAGAAGTTATGTATTCATCCAATAATTCAAGCCAAGCAAGTATATAGGTGTTTT | |
| | AATAGTTTTTGTTTTGGGGATTGTGACCAGAAGATTTTGAAAATTAAATATTAC | |
| | TGAAGATTTCGACTTCCACTGTTAAATGTACAAGATACATGAAATATTAAAGA | |
| | AAATGTGTAACTTTTTGTGTAAATACATCTTGT | |
| 43 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 3 and |
| | CACCTTGTAAAACTGAAGATTGTGACCAGTCAGAATAATGTCCGTGGAATGGT | EWSR1-ATF1 rare |
| | TTGATGCTGCAAGATATGTGCATCTTGCAGCATCAAACCATTCCACGGCATTAT | fusion genes |
| | GGTGACAGCTGCCTCGGGAAGCCAAGTTGGGCTTTAAAGTGCAGGGCCTGCT | |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGTTTTCTTCCCCATTAGGGATTATGCTGAATTTGTATGGTTTA | |
| | TAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGACCAGATCTTGATCTAGTTGCCAGATATGTGCATCTGGCAACTAGATCAA | |
| | GATCTGGTCCATTATGGTGACAGCTGCCTCGGGAAGCCAAGTTGGGCTTTAAA | |
| | GTGCAGGGCCTGCTGATGTTGAGTGCTTTTTGTTCGACAGGATCGATATCTAG | |
| | TCACCAGTGAAGTAGATTAGCATCTGGCAACTAGATCAAGATCTGGTCCATAA | |
| | GAAGTTATGTATTCATCCAATAATTCAAGCCAAGCAAGTATATAGGTGTTTTAA | |
| | TAGTTTTTGTTTTGGGGATTGTGACCAGAAGATTTTGAAAATTAAATATTACTG | |
| | AAGATTTCGACTTCCACTGTTAAATGTACAAGATACATGAAATATTAAAGAAA | |
| | ATGTGTAACTTTTTGTGTAAATACATCTTGT | |
| 44 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 4 and |
| | CACCTTGTAAAACTGAAGATTGTGACCAGTCAGAATAATGTCCGTGGAATGGT | EWSR1-ATF1 rare |
| | TTGATGCTGCAAGATATGTGCATCTTGCAGCATCAAACCATTCCACGGCATTAT | fusion genes |
| | GGTGACAGCTGCCTCGGGAAGCCAAGTTGGGCTTTAAAGTGCAGGGCCTGCT | |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGTTTTCTTCCCCATTAGGGATTATGCTGAATTTGTATGGTTTA | |
| | TAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGCAGCTACGGGCAGCAGAAGAAGAGATATGTGCATCTCTTCTTCTGCTGC | |
| | CCGTAGCTGCCATTATGGTGACAGCTGCCTCGGGAAGCCAAGTTGGGCTTTAA | |
| | AGTGCAGGGCCTGCTGATGTTGAGTGCTTTTTGTTCGCACATACGATGAGCAG | |
| | ACTAAGAGTGAAGTAGATTAGCATCTCTTCTTCTGCTGCCCGTAGCTGCCATA | |
| | AGAAGTTATGTATTCATCCAATAATTCAAGCCAAGCAAGTATATAGGTGTTTTA | |
| | ATAGTTTTTGTTTTGGGGATTGTGACCAGAAGATTTTGAAAATTAAATATTACT | |
| | GAAGATTTCGACTTCCACTGTTAAATGTACAAGATACATGAAATATTAAAGAA | |
| | AATGTGTAACTTTTTGTGTAAATACATCTTGT | |
| 45 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting the |
| | CACCTTGTAAAACTGAAGATTGTGACCAGTCAGAATAATGTGCAGCTACGGG | EWSR1-CREB1 |
| | CAGCAGATTGCCAGATATGTGCATCTGGCAATCTGCTGCCCGTAGCTGCCATTA | fusion gene |
| | TGGTGACAGCTGCCTCGGGAAGCCAAGTTGGGCTTTAAAGTGCAGGGCCTGC | |
| | TGATGTTGAGTGCTTTTTGTTCGCAGACACGAGTAGCAGATCACCAGTGAAGT | |
| | AGATTAGCATCTGGCAATCTGCTGCCCGTAGCTGCCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGT | |
| 46 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 1 |
| | CACCTTGTAAAACTGAAGATTGTGACCAGTCAGAATAATGTAGGACGCGGTG | EWSR1-ATF1 |
| | GAATGGGAAAAAAGATATGTGCATCTTTTTTCCCATTCCACCGCGTCCTCATTA | fusion gene and |
| | TGGTGACAGCTGCCTCGGGAAGCCAAGTTGGGCTTTAAAGTGCAGGGCCTGC | EWSR-CREB1 |
| | TGATGTTGAGTGCTTTTTGTTCAGGACGATGAACAATGGGTGAAAAGTGAAG | fusion gene |
| | TAGATTAGCATCTTTTTTCCCATTCCACCGCGTCCTCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGCAGCTACGGGCAGCAGATTGCCAGATATGTGCATCTGGCAATCTGCT | |
| | GCCCGTAGCTGCCATTATGGTGACAGCTGCCTCGGGAAGCCAAGTTGGGCTTT | |
| | AAAGTGCAGGGCCTGCTGATGTTGAGTGCTTTTTGTTCGCAGACACGAGTAG | |
| | CAGATCACCAGTGAAGTAGATTAGCATCTGGCAATCTGCTGCCCGTAGCTGCC | |
| | ATAAGAAGTTATGTATTCATCCAATAATTCAAGCCAAGCAAGTATATAGGTGTT | |
| | TTAATAGTTTTTGTTTTGGGGATTGTGACCAGAAGATTTTGAAAATTAAATATT | |
| | ACTGAAGATTTCGACTTCCACTGTTAAATGTACAAGATACATGAAATATTAAAG | |
| | AAAATGTGTAACTTTTTGTGTAAATACATCTTGT | |
| 47 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 2 |
| | CACCTTGTAAAACTGAAGATTGTGACCAGTCAGAATAATGTGCAGCTACGGG | EWSR1-ATF1 |
| | CAGCAGATTGCCAGATATGTGCATCTGGCAATCTGCTGCCCGTAGCTGCCATTA | fusion gene and |
| | TGGTGACAGCTGCCTCGGGAAGCCAAGTTGGGCTTTAAAGTGCAGGGCCTGC | EWSR-CREB1 |
| | TGATGTTGAGTGCTTTTTGTTCGCAGACACGAGTAGCAGATCACCAGTGAAGT | fusion gene |
| | AGATTAGCATCTGGCAATCTGCTGCCCGTAGCTGCCATAAGAAGTTATGTATTC | |
| | ATCCAATAATTCAAGCCAAGCAAGTATATAGGTGTTTTAATAGTTTTTGTTTTG | |
| | GGGATTGTGACCAGAAGATTTTGAAAATTAAATATTACTGAAGATTTCGACTT | |
| | CCACTGTTAAATGTACAAGATACATGAAATATTAAAGAAAATGTGTAACTTTTT | |
| | GTGTAAATACATCTTGTTTTCTTCCCCATTAGGGATTATGCTGAATTTGTATGGT | |
| | TTATAGTTGTTAGAGTTTGAGGTGTTAATTCTAATTATCTATTTCAAATTTAGCA | |
| | GGAAAAAAGAGAACATCACCTTGTAAAACTGAAGATTGTGACCAGTCAGAAT | |
| | AATGTGCAGCTACGGGCAGCAGATTGCCAGATATGTGCATCTGGCAATCTGCT | |
| | GCCCGTAGCTGCCATTATGGTGACAGCTGCCTCGGGAAGCCAAGTTGGGCTTT | |
| | AAAGTGCAGGGCCTGCTGATGTTGAGTGCTTTTTGTTCGCAGACACGAGTAG | |
| | CAGATCACCAGTGAAGTAGATTAGCATCTGGCAATCTGCTGCCCGTAGCTGCC | |
| | ATAAGAAGTTATGTATTCATCCAATAATTCAAGCCAAGCAAGTATATAGGTGTT | |
| | TTAATAGTTTTTGTTTTGGGGATTGTGACCAGAAGATTTTGAAAATTAAATATT | |
| | ACTGAAGATTTCGACTTCCACTGTTAAATGTACAAGATACATGAAATATTAAAG | |
| | AAAATGTGTAACTTTTTGTGTAAATACATCTTGT | |
| 48 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 3 |
| | CACCTTGTAAAACTGAAGATTGTGACCAGTCAGAATAATGTGACCAGATCTTG | EWSR1-ATF1 |
| | ATCTAGTTGCCAGATATGTGCATCTGGCAACTAGATCAAGATCTGGTCCATTAT | fusion gene and |
| | GGTGACAGCTGCCTCGGGAAGCCAAGTTGGGCTTTAAAGTGCAGGGCCTGCT | EWSR-CREB1 |
| | GATGTTGAGTGCTTTTTGTTCGACAGGATCGATATCTAGTCACCAGTGAAGTA | fusion gene |
| | GATTAGCATCTGGCAACTAGATCAAGATCTGGTCCATAAGAAGTTATGTATTCA | |
| | TCCAATAATTCAAGCCAAGCAAGTATATAGGTGTTTTAATAGTTTTTGTTTTGG | |
| | GGATTGTGACCAGAAGATTTTGAAAATTAAATATTACTGAAGATTTCGACTTCC | |
| | ACTGTTAAATGTACAAGATACATGAAATATTAAAGAAAATGTGTAACTTTTTGT | |
| | GTAAATACATCTTGTTTTCTTCCCCATTAGGGATTATGCTGAATTTGTATGGTTT | |
| | ATAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGCAGCTACGGGCAGCAGATTGCCAGATATGTGCATCTGGCAATCTGCTGC | |
| | CCGTAGCTGCCATTATGGTGACAGCTGCCTCGGGAAGCCAAGTTGGGCTTTAA | |
| | AGTGCAGGGCCTGCTGATGTTGAGTGCTTTTTGTTCGCAGACACGAGTAGCA | |
| | GATCACCAGTGAAGTAGATTAGCATCTGGCAATCTGCTGCCCGTAGCTGCCAT | |
| | AAGAAGTTATGTATTCATCCAATAATTCAAGCCAAGCAAGTATATAGGTGTTTT | |
| | AATAGTTTTTGTTTTGGGGATTGTGACCAGAAGATTTTGAAAATTAAATATTAC | |
| | TGAAGATTTCGACTTCCACTGTTAAATGTACAAGATACATGAAATATTAAAGA | |
| | AAATGTGTAACTTTTTGTGTAAATACATCTTGT | |
| 49 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting Type 4 |
| | CACCTTGTAAAACTGAAGATTGTGACCAGTCAGAATAATGTGCAGCTACGGG | EWSR1-ATF1 |
| | CAGCAGAAGAAGAGATATGTGCATCTCTTCTTCTGCTGCCCGTAGCTGCCATT | fusion gene and |
| | ATGGTGACAGCTGCCTCGGGAAGCCAAGTTGGGCTTTAAAGTGCAGGGCCTG | EWSR-CREB1 |
| | CTGATGTTGAGTGCTTTTTGTTCGCACATACGATGAGCAGACTAAGAGTGAAG | fusion gene |
| | TAGATTAGCATCTCTTCTTCTGCTGCCCGTAGCTGCCATAAGAAGTTATGTATT | |
| | CATCCAATAATTCAAGCCAAGCAAGTATATAGGTGTTTTAATAGTTTTTGTTTT | |
| | GGGGATTGTGACCAGAAGATTTTGAAAATTAAATATTACTGAAGATTTCGACT | |
| | TCCACTGTTAAATGTACAAGATACATGAAATATTAAAGAAAATGTGTAACTTTT | |
| | TGTGTAAATACATCTTGTTTTCTTCCCCATTAGGGATTATGCTGAATTTGTATGG | |
| | TTTATAGTTGTTAGAGTTTGAGGTGTTAATTCTAATTATCTATTTCAAATTTAGC | |
| | AGGAAAAAAGAGAACATCACCTTGTAAAACTGAAGATTGTGACCAGTCAGA | |
| | ATAATGTGCAGCTACGGGCAGCAGATTGCCAGATATGTGCATCTGGCAATCTG | |
| | CTGCCCGTAGCTGCCATTATGGTGACAGCTGCCTCGGGAAGCCAAGTTGGGC | |
| | TTTAAAGTGCAGGGCCTGCTGATGTTGAGTGCTTTTTGTTCGCAGACACGAGT | |
| | AGCAGATCACCAGTGAAGTAGATTAGCATCTGGCAATCTGCTGCCCGTAGCTG | |
| | CCATAAGAAGTTATGTATTCATCCAATAATTCAAGCCAAGCAAGTATATAGGTG | |
| | TTTTAATAGTTTTTGTTTTGGGGATTGTGACCAGAAGATTTTGAAAATTAAATA | |
| | TTACTGAAGATTTCGACTTCCACTGTTAAATGTACAAGATACATGAAATATTAA | |
| | AGAAAATGTGTAACTTTTTGTGTAAATACATCTTGT | |
| 50 | TTTCTTCCCCATTAGGGATTATGCTGAATTTGTATGGTTTATAGTTGTTAGAGTT | bi-shRNA sequence |
| | TGAGGTGTTAATTCTAATTATCTATTTCAAATTTAGCAGGAAAAAAGAGAACAT | targeting EWSRI- |
| | CACCTTGTAAAACTGAAGATTGTGACCAGTCAGAATAATGTCCGTGGAATGGT | ATF1 rare fusion |
| | TTGATGCTGCAAGATATGTGCATCTTGCAGCATCAAACCATTCCACGGCATTAT | gene and EWSR1- |
| | GGTGACAGCTGCCTCGGGAAGCCAAGTTGGGCTTTAAAGTGCAGGGCCTGCT | CREB1 fusion gene |
| | GATGTTGAGTGCTTTTTGTTCCCATGTAATGACCTGATGATACAAGTGAAGTAG | |
| | ATTAGCATCTTGCAGCATCAAACCATTCCACGGCATAAGAAGTTATGTATTCAT | |
| | CCAATAATTCAAGCCAAGCAAGTATATAGGTGTTTTAATAGTTTTTGTTTTGGG | |
| | GATTGTGACCAGAAGATTTTGAAAATTAAATATTACTGAAGATTTCGACTTCCA | |
| | CTGTTAAATGTACAAGATACATGAAATATTAAAGAAAATGTGTAACTTTTTGTG | |
| | TAAATACATCTTGTTTTCTTCCCCATTAGGGATTATGCTGAATTTGTATGGTTTA | |
| | TAGTTGTTAGAGTTTGAGGTGTTAATTCTAATTATCTATTTCAAATTTAGCAGG | |
| | AAAAAAGAGAACATCACCTTGTAAAACTGAAGATTGTGACCAGTCAGAATAA | |
| | TGTGCAGCTACGGGCAGCAGATTGCCAGATATGTGCATCTGGCAATCTGCTGC | |
| | CCGTAGCTGCCATTATGGTGACAGCTGCCTCGGGAAGCCAAGTTGGGCTTTAA | |
| | AGTGCAGGGCCTGCTGATGTTGAGTGCTTTTTGTTCGCAGACACGAGTAGCA | |
| | GATCACCAGTGAAGTAGATTAGCATCTGGCAATCTGCTGCCCGTAGCTGCCAT | |
| | AAGAAGTTATGTATTCATCCAATAATTCAAGCCAAGCAAGTATATAGGTGTTTT | |
| | AATAGTTTTTGTTTTGGGGATTGTGACCAGAAGATTTTGAAAATTAAATATTAC | |
| | TGAAGATTTCGACTTCCACTGTTAAATGTACAAGATACATGAAATATTAAAGA | |
| | AAATGTGTAACTTTTTGTGTAAATACATCTTGT | |
| 51 | CGTGGAATGGTTTGATGCTGCATC | Targeting region in |
| | | EWSR1-ATF1 rare |
| | | fusion gene of |
| | | pGBI-2003 |
| 52 | CCGTGGAATGGTTTGATGCTGCAT | Targeting region in |
| | | EWSR1-ATF1 rare |
| | | fusion gene of |
| | | pGBI-2004 |
| 53 | GCCGTGGAATGGTTTGATGCTGCA | Targeting region in |
| | | EWSR1-ATF1 rare |
| | | fusion gene of |
| | | pGBI-2005 |
| 54 | TGCCGTGGAATGGTTTGATGCTGC | Targeting region in |
| | | EWSR1-ATF1 rare |
| | | fusion gene of |
| | | pGBI-2006 |
| 55 | CTGCCGTGGAATGGTTTGATGCTG | Targeting region in |
| | | EWSR1-ATF1 rare |
| | | fusion gene of |
| | | pGBI-2007 |
| 56 | TGCCGTGGAATGGTTTGATGCTGC | Targeting region in |
| | | EWSR1-ATF1 rare |
| | | fusion gene of |
| | | pGBI-2008 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An expression vector comprising a bifunctional short hairpin RNA (bi-shRNA) sequence specific for knockdown of at least one EWSR1-ATF1 fusion gene and/or an EWSR1-CREB1 fusion gene, wherein the bi-shRNA sequence encodes a nucleic acid sequence capable of hybridizing to a target region of an mRNA transcript encoding the EWSR1-ATF1 fusion gene and/or a target region of an mRNA transcript encoding the EWSR1-CREB1 fusion gene to inhibit the expression of the EWSR1-ATF1 fusion gene and/or the EWSR1-CREB1 fusion gene via RNA interference,
wherein the bi-shRNA comprises a first stem-loop structure that comprises an siRNA component and a second stem-loop structure that comprises a miRNA component.

2. The expression vector of claim 1, wherein the siRNA component functions in a cleavage-dependent manner and the miRNA component functions in a cleavage-independent manner.

3. The expression vector of claim 1, wherein the bi-shRNA sequence is operably linked to a promoter.

4. The expression vector of claim 3, wherein the promoter is a CMV mammalian promoter.

5. (canceled)

6. The expression vector of claim 1, wherein the bi-shRNA sequence is specific for knockdown of:
a) at least one EWSR1-ATF1 fusion gene,
b) two EWSR1-ATF1 fusion genes;
c) three EWSR1-ATF1 fusion genes;
d) an EWSR1-CREB1 fusion gene; or
e) one EWSR1-ATF1 fusion gene and one EWSR1-CREB1 fusion gene.

7.-15. (canceled)

16. A composition comprising a therapeutic agent carrier and an expression vector of claim 1.

17. The composition of claim 16, wherein the therapeutic agent carrier is a compacted DNA nanoparticle.

18. The composition of claim 17, wherein the DNA nanoparticle is compacted with one or more polycations.

19. The composition of claim 18, wherein the compacted DNA nanoparticles are further encapsulated in a liposome.

20. The composition of claim 16, wherein the therapeutic agent carrier is a liposome.

21. The composition of claim 20, wherein the liposome is a bilamellar invaginated vesicle (BIV).

22. The composition of claim 20, wherein the liposome is a reversibly masked liposome.

23. The composition of claim 20, wherein the liposome is decorated with one or more receptor targeting moieties.

24. The composition of claim 20, wherein the liposome comprises DOTAP and cholesterol.

25. A method for delivering one or more bi-shRNAs to a target tissue expressing at least one EWSR1-ATF1 fusion gene and/or an EWSR1-CREB1 fusion gene, the method comprises the steps of:
(a) preparing an expression vector of claim 1;
(b) combining the expression vector with a therapeutic agent carrier; and
(c) administering a therapeutically effective amount of the expression vector and therapeutic agent carrier complex to a subject in need thereof.

26. The method of claim 25, wherein the therapeutic agent carrier is a compacted DNA nanoparticle or a liposome.

27. A method to inhibit an expression of at least one EWSR1-ATF1 fusion gene and/or an EWSR1-CREB1 fusion gene in one or more target cells comprising the steps of:
(a) selecting the one or more target cells; and
(b) transfecting the one or more target cells with an expression vector of claim 1.

28. The method of claim 27, wherein the target cells are cancer cells or tumor cells.

29. (canceled)

30. A method of suppressing a tumor cell growth in a subject comprising the steps of:
(a) identifying the subject in need for suppression of the tumor cell growth; and
(b) administering an expression vector of claim 1,
wherein the inhibition results in an apoptosis, an arrested proliferation, or a reduced invasiveness of the tumor cells.

31.-38. (canceled)

39. The expression vector of claim 6, wherein:
a) the bi-shRNA sequence specific for knockdown of one EWSR1-ATF1 fusion gene comprises a sequence having at least 90% identity to any one of SEQ ID NOS:21 to 26;
b) the bi-shRNA sequence specific for knockdown of two EWSR1-ATF1 fusion genes comprises a sequence having at least 90% identity to any one of SEQ ID NOS:35 to 44;
c) the bi-shRNA sequence specific for knockdown of three EWSR1-ATF1 fusion genes comprises a sequence having at least 90% identity to any one of SEQ ID NOS:27-30;
d) the bi-shRNA sequence specific for knockdown of an EWSR1-CREB1 fusion gene comprises a sequence having at least 90% identity to SEQ ID NO:45; or
e) the bi-shRNA sequence specific for knockdown of one EWSR1-ATF1 fusion gene and one EWSR1-CREB1 fusion gene comprises a sequence having at least 90% identity to any one of SEQ ID NOS:46 to 50.
